(19) United States
(12) Patent Application Publication
(10) Pub. No.: US 2007/0190068 A1
(43) Pub. Date: Aug. 16, 2007

(54) uPAR-binding molecule-drug conjugates and uses thereof

(76) Inventors: Richard Hart, Greenwich, CT (US); Shafaat A. Rabbani, Westmont (CA)

Correspondence Address: JONES DAY, 222 EAST 41ST ST, NEW YORK, NY, 10017, US

(21) Appl. No.: 11/546,582

(22) Filed: Oct. 10, 2006

(30) Foreign Application Priority Data
Oct. 10, 2005 (EP) 05 022 040.9

(51) Int. Cl. A61K 39/395

(52) U.S. Cl. 424/179.1; 424/178.1

(57) ABSTRACT

The present invention relates to the use of uPAR-binding molecule-drug conjugates capable of specifically binding a urokinase plasminogen activator receptor (uPAR) as therapeutic and diagnostic reagents for the treatment and monitoring of metastases. The present invention provides methods of treatment of metastases, comprising administering to a subject a uPAR-binding molecule-chemotherapeutic conjugate that is capable of binding to and internalizing into uPAR-expressing cells. The present invention further provides pharmaceutical compositions and kits comprising such conjugates. The present invention further provides methods and compositions relating to combination therapy for cancer involving or mediated by uPAR-expressing cells using uPAR-binding molecule-drug conjugates of the invention.

This application claims the benefit of European Patent Application 05 022 040.9, filed Oct. 10, 2005, which is incorporated herein by reference in its entirety.

### 1. FIELD OF THE INVENTION

The present invention relates to uPAR-binding molecule-drug conjugates that are capable of specifically binding a urokinase plasminogen activator receptor (uPAR) as therapeutic and diagnostic reagents. The present invention provides methods of treatment of metastases, comprising administering to a subject a uPAR-binding molecule-drug conjugate that is capable of binding to and internalizing into uPAR-expressing cells. The present invention further provides pharmaceutical compositions and kits comprising such conjugates. The present invention also provides methods of diagnosis using the conjugates of the present invention. The present invention further provides methods and compositions relating to combination therapy for metastases involving or mediated by uPAR-expressing cells using uPAR-binding molecule-drug conjugates of the invention.

### 2. BACKGROUND OF THE INVENTION

Cancer is characterized primarily by an increase in the number of abnormal cells derived from a given normal tissue, invasion of adjacent tissues by these abnormal cells, and lymphatic or blood-borne spread of malignant cells to regional lymph nodes and to distant sites (metastasis). Clinical data and molecular biological studies indicate that cancer is a multistep process that begins with minor preneoplastic changes, which may under certain conditions progress to neoplasia. Metastasis, the growth of secondary tumors at sites distant from a primary tumor, is the major cause of failures of cancer treatment.

The regulatory mechanisms involved in metastases differ from those that cause tumor formation. In fact, metastatic cells appear to be physiologically different than tumor cells. For example, metastatic cells differ in expression of genes such as ras oncogene, serine-threonine kinases, tyrosine kinases, and p53 as well as differ in signal transduction (for review see Liotta et al., 1991, Cell 64:327-336).

Prior to metastasis, expansion of a tumor involves angiogenesis, the formation of new blood vessels (Folkman et al., 1989, Nature 339:58-61). Tumors have been shown to induce angiogenesis through several soluble factors (Folkman et al., 1987, Science 235:442-447; Pepper et al., 1990, J. Cell Biol. 111:743-755). Angiogenesis is a multistep process emanating from microvascular endothelial cells. Endothelial cells resting in parent vessels are stimulated to degrade the endothelial basement membrane, migrate into the perivascular stroma, and initiate a capillary sprout (Liotta et al., 1991, Cell 64:327-336). The capillary sprout subsequently expands and assumes a tubular structure. Endothelial proliferation leads to extension of the microvascular tubules, which develop into loops and then into a functioning circulatory network. The exit of endothelial cells from the parent vessel involves cell migration and degradation of the extracellular matrix (ECM) in a manner similar to cancer cell invasion of the ECM (Liotta et al., 1991, Cell 64:327-336).

Cancer cell invasion involves interactions of cancer cells with the ECM, a dense latticework of collagen and elastin embedded in a gel-like ground substance composed of proteoglycans and glycoproteins. The ECM consists of the basement membrane and its underlying interstitial stroma. Tumor invasion involves: (1) cancer cell detachment from their original location; (2) attachment to the ECM; (3) degradation of the ECM; and (4) locomotion into the ECM (for review see Liotta, 1986, Cancer Res. 46:1-7). Following detachment of the cancer cells, the cells migrate over the ECM and adhere to components of the ECM such as laminin, type IV collagen and fibronectin via cell surface receptors. Cell adhesion molecules, such as integrin, have been shown to mediate cancer cell attachment to vascular endothelial cells and to matrix proteins (Mundy, 1997, Cancer 80(9):1546-1556). The attached cancer cell then secretes hydrolytic enzymes or induces host cells to secrete enzymes which locally degrade the matrix. Matrix lysis occurs in a highly localized region close to the cancer cell surface, where the amount of active enzyme outbalances the natural proteinase inhibitors present in the serum, in the matrix, or that secreted by normal cells in the vicinity (Liotta et al., 1991, Cell 64:327-336). A positive association with tumor aggressiveness has been noted for various classes of degradative enzymes, including: heparinases, thiol-proteinases (including cathepsins B and L), metalloproteinases (including collagenases, gelatinases, and stromelysins), and serine proteinases (including plasmin and urokinase plasminogen activator).

During the locomotion step of invasion, cancer cells migrate across the basement membrane and stroma through the zone of matrix proteolysis. The cancer cells then enter tumor capillaries (which arise as a consequence of specific angiogenic factors) and reach the general circulation via these capillaries. After traveling to distant sites of the organism, the intravasated cancer cells adhere to and extravasate through the vascular endothelium, and initiate new tumor formation, i.e., first forming a mass of cells that, via the angiogenesis process, becomes a vascularized tumor.

Thus, metastasis is not a simple, random process but rather is a multistep process dependent on specific properties of the tumor cells and supportive factors in the environment of the metastatic site.

A large number of different molecules are involved in the metastatic process. Two examples of such molecules are uPA and its receptor, uPAR, which have been implicated in the tumor cell invasion aspect of the metastatic process. During cancer invasion, uPAR binds uPA released from surrounding cancer or stroma cells. Binding of uPA to its receptor focuses proteolytic action to the surface of cancer cells. uPA converts enzymatically inactive plasminogen into the serine protease, plasmin. Plasmin degrades many ECM proteins such as fibronectin, vitronectin, and fibrin thus facilitating ECM degradation, cancer cell proliferation, invasion, and metastasis (Schmitt et al., 1997, Thrombosis and Haemostasis 78(1):285-296). Plasmin can also catalyze activation of the zymogen forms of several metalloproteinases.

A critical balance of urokinase-type plasminogen activator (uPA), its cell surface receptor uPAR, and its inhibitor, plasminogen activator inhibitor-1 (PAI-1) is the prerequisite for efficient focal proteolysis, adhesion and migration, and hence, subsequent tumor cell invasion and metastasis. (Andreasen, et al., 1997, Int. Journal Cancer 72: 1-22; Schmitt, et al., 1997, Thrombosis Haemostasis 78: 285-296).

Urokinase plasminogen activator receptor (uPAR) is a 313 residue protein with a 282 residues hydrophilic N terminal portion (probably extracellular) followed by 21 hydrophobic amino acids (probably trans-membrane domain). The potential extracellular domain is organized in three highly homologous repeats. The precursor protein further contains 22 amino acid residues of signaling peptide. Roldan et al., 1990, EMBO 9(2):467-474. Some of the u-PAR are terminally processed and are anchored to the cell surface. Some uPAR are not anchored and are free receptors in serum. It is possible that measurement of free receptor may be a diagnostically valuable indicator of some pathological processes. See U.S. Pat. No. 5,519,120. The high numbers of uPAR on the surface of cancer cells, if occupied by Urokinase plasminogen activator (uPA), create elevated proteolytic activity in the proximity of cancer cells and hence allow dissolution of surrounding tissue which facilitate cancer invasion. Kwaan et al., 1991, Sem. Throm. Hemo. 17:175-182. To a lesser extent, elevated levels of uPAR may also indicate poor prognosis (Schmitt et al., Thrombosis and Haemostasis 78(1):285-296). The important role of uPA-uPAR in tumor growth and its abundant expression within tumor, but not normal tissue, makes this system an attractive diagnostic and therapeutic agent.

Several studies have been conducted to examine the therapeutic effect of substances that interact with components of the plasminogen activation pathway. Manipulation of the plasminogen activation pathway has resulted in decreased tumor growth rates (Jankun et al., U.S. Pat. No. 5,679,350 (injection of a medicament coupled to PAI-1 or PAI-2); anti-uPA antibodies decrease tumor cell invasion and/or metastasis of cells from cultured tumor cell lines transplanted into animal models (for review seen Andreasen et al., 1997, Int. J. Cancer 72:1-22); Dano et al., U.S. Pat. No. 5,519,120 (injection of anti-uPA or anti-uPAR antibodies); and Xing and Rabbani, 1996, Proc. Amer. Assoc. Cancer Res. 37:90 (Abstract #626) (injection of anti-uPAR antibodies)). There are also studies relating to the diagnosis of metastases using urokinase plasminogen activator as a target, See U.S. Pat. No. 6,077,508.

Previous efforts have been involved with the use of substances that inhibit the interaction of uPA and uPAR for the treatment of pathological states, such as cancer. Other effective approach for treatment of metastases involves therapeutics that do not interfere or inhibit the interaction between uPA and uPAR. However, these therapeutic agents require very high doses, in part, due to rapid clearance of the therapeutic from the system once it is administered. Possible reasons for the ineffectiveness of these therapeutics includes dilution of the therapeutics in the blood stream before reaching the target cells or short half-life of these therapeutics due to degradation of the therapeutics in vivo. Also, there is a lack of an effective therapeutic that specifically treat, ameliorate or prevent metastasis involving uPAR-expressing cells. There is a need for a therapeutic that is capable of binding to and being internalized into the target cells expressing uPAR. Such compounds would be useful therapeutic agents against metastases that involve cells expressing uPAR.

Citation or identification of any reference herein shall not be construed as an admission that such reference is available as prior art to the present invention.

### 3. SUMMARY OF THE INVENTION

The present invention provides uPAR-binding molecule-drug conjugate molecules comprising a uPAR-binding molecule that is conjugated to a drug. The uPAR-binding molecule-drug conjugate molecule is capable of accumulating in uPAR-expressing cells. Upon administration to a patient, the conjugate molecules bind to uPAR on the target cells through their uPAR-binding molecule portion and become internalized, allowing the drug to exert its toxic effects in the target cells.

In certain embodiments, the uPAR-binding molecule of a uPAR-binding molecule-drug conjugate molecule of the invention is a peptide, derivative or analog thereof that binds specifically to uPAR such as peptides derived from uPA. In preferred embodiments, the uPAR-binding molecule is capable of being internalized into the uPAR-expressing cells.

In specific embodiments, the uPAR-binding molecule is an anti-uPAR antibody or fragments thereof. Recombinant antibody fragments includes Fabs that are composed of the light chain and the heavy chain Fd fragment (VH and CH1), connected to each other via the interchain disulfide bond between CL and CH1. The invention also includes ScFv fragments stabilized by a peptide linker which connects the carboxyl-terminus of VH or VL with the amino terminus of the other domain. The VH and VL heterodimer in dsFv is stabilized by further engineering a disulfide bond between the two domains. In certain embodiments, the uPAR-binding molecule of a uPAR-binding molecule-drug conjugate of the invention is a monoclonal antibody, a humanized chimeric antibody, a chimeric antibody, a humanized antibody, a glycosylated antibody, a multispecific antibody, a human antibody, a single-chain antibody, a Fab fragment, a F(ab′) fragment, a F(ab′)₂ fragment, a Fd, a single-chain Fv, a disulfide-linked Fv, a fragment comprising a V_{L} domain, or a fragment comprising a V_{H} domain. In certain embodiments, the antibody is a bispecific antibody. In other embodiments, the antibody is not a bispecific antibody.

In preferred embodiments, the uPAR-binding molecule-drug conjugate is antibody 3936 conjugated to doxorubicin.

In preferred embodiments, the drug is a chemotherapeutic agent. The chemotherapeutic agent is a selectively cytotoxic agent or a cytostatic agent which selectively kills or inhibits the growth of cancer cells.

In certain embodiments, the uPAR-binding molecule of the uPAR-binding molecule-drug conjugate is conjugated indirectly to a drug through a protein or a peptide. In specific embodiments, the protein is an inhibitor of the uPAR-binding molecule. In specific embodiments, the inhibitor of the uPAR-binding molecule is PAI-1 or PAI-2. In certain embodiment, the uPAR-binding molecule-drug conjugate comprises a uPAR-binding molecule conjugated to an inhibitor of the uPAR-binding molecule, said inhibitor of the uPAR-binding molecule is conjugated to a drug.

In certain embodiments, the present invention is directed to a conjugate molecule comprising a uPA inhibitor conjugated to a drug.

In a specific embodiment, the present invention is directed to a conjugate molecule comprising a PAI-1 conjugated to doxorubicin. In another specific embodiment, the conjugate molecule comprising a PAI-2 conjugated to doxorubicin.

In certain embodiments, the uPAR-binding molecule of the uPAR-binding molecule-drug conjugate is radioactively labeled.

The invention further provides a uPAR-binding molecule-drug conjugate, wherein the conjugate has a rate of accumulation in a uPAR-expressing cell that is at least 20 to 40, 40 to 60, 60 to 80, 80 to 100, 100-200, 200-500, 500 to 1,000, 1,000 to 2,000, 2,000 to 2,500 fold greater than the rate of accumulation of an unconjugated form of the drug in the uPAR-expressing cell, wherein the rates of accumulation of the conjugate and of an unconjugated form of the drug are measured by a method comprising: (a) culturing a population of the uPAR-expressing cell with the conjugate; (b) culturing a population of the uPAR-expressing cell with an unconjugated form of the drug, wherein the populations of steps (a) and (b) are cultured under the same conditions; and (c) measuring the amount of the conjugate and an unconjugated form of the drug accumulated in the populations of the uPAR-expressing cells in steps (a) and (b), respectively.

In certain embodiments, the rates of accumulation of the conjugate and an unconjugated form of the drug in the uPAR-expressing cell are determined by: (a) culturing a population of the uPAR-expressing cell in the presence of the conjugate, wherein the conjugate is labeled with a radioactive isotope; (b) culturing a population of the uPAR-expressing cell with an unconjugated form of the drug under the same conditions as the culturing of step (a), wherein the unconjugated form of the drug is labeled with the radioactive isotope; and (c) comparing the amount of the radioactive isotope in the populations of uPAR-expressing cells in steps (a) and (b), wherein the rate of accumulation of the conjugate in the uPAR-expressing cell is at least 20 to 40, 40 to 60, 60 to 80, 80 to 100, 100-200, 200-500, 500 to 1,000, 1,000 to 2,000, 2,000 to 2,500 folds greater than the rate of accumulation of an unconjugated form of the drug in the uPAR-expressing cell if the amount of the radioactive isotope in the population of uPAR-expressing cells in step (a) is at least 20 to 40, 40 to 60, 60 to 80, 80 to 100, 100-200, 200-500, 500 to 1,000, 1,000 to 2,000, 2,000 to 2,500 folds greater than the amount of the radioactive isotope in the population of uPAR-expressing cells in step (b).

The present invention further provides pharmaceutical compositions and kits comprising such conjugate molecules.

The invention further provides, a pharmaceutical composition comprising a uPAR-binding molecule-drug conjugate, wherein the conjugate has a rate of accumulation in a uPAR-expressing cell that is at least 20 to 40, 40 to 60, 60 to 80, 80 to 100, 100-200, 200-500, 500 to 1,000, 1,000 to 2,000, 2,000 to 2,500 folds greater than the rate of accumulation of an unconjugated form of the anti-uPAR antibody in the uPAR-expressing cell, wherein the rates of accumulation of the conjugate and of the unconjugated form of the antibody are measured by a method comprising: (a) culturing a population of the uPAR-expressing cell with the conjugate; (b) culturing a population of the uPAR-expressing cell with an unconjugated form of the drug, wherein the populations of steps (a) and (b) are cultured under the same conditions; and (c) measuring the amount of the conjugate and unconjugated antibody accumulated in the populations of steps (a) and (b), respectively.

The present invention also provides the methods of using the uPAR-binding molecule-drug conjugates of the invention.

The present invention relates to methods for the treatment, amelioration or prevention of primary tumors and metastases expressing uPAR using the conjugates of the invention that specifically bind cells expressing uPAR. The present invention further provides methods of treatment of metastases involving uPAR-expressing cells, comprising administering to a patient in need of such treatment a uPAR-binding molecule-drug conjugate of the invention, in either single therapy or combination therapy regimens.

The invention further provides a method of treating metastases involving uPAR-expressing cells, comprising administering to a subject in need of such treatment an effective amount of a uPAR-binding molecule-drug conjugate, wherein the conjugate has a rate of accumulation in a uPAR-expressing cell that is at least 20 to 40, 40 to 60, 60 to 80, 80 to 100, 100-200, 200-500, 500 to 1,000, 1,000 to 2,000, 2,000 to 2,500 folds greater than the rate of accumulation of an unconjugated form of the drug in the uPAR-expressing cell, wherein the rates of accumulation of the conjugate and of the unconjugated form of the antibody are measured by a method comprising: (a) culturing a population of the uPAR-expressing cell with the conjugate; (b) culturing a population of the uPAR-expressing cell with the unconjugated drug, wherein the populations of steps (a) and (b) are cultured under the same conditions; and (c) measuring the amount of the conjugate and unconjugated drug accumulated in the populations of steps (a) and (b), respectively. In specific embodiments, the uPAR-expressing cells are of the same cell type in steps (a) and (b).

In certain embodiments, the methods of the invention for treating metastases involving uPAR-expressing cells further comprise administering to the subject a second therapeutic agent. In certain embodiments, the therapeutic agent is a cytostatic or cytotoxic agent.

The present invention also relates to methods of detecting uPAR polypeptide in a sample using labeled conjugates of the present invention. The present invention also relates to methods of diagnosis and imaging of primary tumors and of metastases using labeled conjugates of the present invention. A particular embodiment of the present invention is the use of the conjugates of the present invention for detecting and imaging metastases in vivo. By introducing an aliquot of the labeled conjugate (e.g., radiolabeled conjugate) into the medication being administered, direct imaging may be performed during the treatment process.

Metastatic tumors, while derived from cells of the primary tumor, are considerably altered in their physiologic and growth characteristics and need not express the same surface markers as parental primary tumors. The conjugates of the present invention can be used to detect free uPAR in a sample, uPAR-expressing cells in a tumor, and u-PAR-expressing cells distal to the primary tumor, which are engaged in establishing new tumors (i.e., via attachment, not mobilization, cell expansion, angiogenesis, etc.).

In a preferred embodiment of the invention, metastases in a subject are detected by: (a) administering labeled conjugate which specifically bind uPAR; (b) permitting the labeled conjugate to preferentially concentrate in one or more metastatic lesions in the subject and unbound labeled conjugate to be cleared to background level; (c) determining the background level; and (d) detecting the labeled conjugate such that detection of labeled conjugate above the background level indicates the presence of a metastatic lesion.

In another preferred embodiment, the labeled molecule of the invention can be detected in a subject wherein the subject had been administered the labeled conjugate at a sufficient time interval prior to detection to allow the labeled conjugate to preferentially concentrate at metastatic lesions.

The present invention further provides kits comprising a uPAR-binding molecule-drug conjugate of the invention. Optionally, the kits may further comprise one or more additional therapeutic agents as described in Sections 5.1.5. Exemplary embodiments of the kits of the invention are described below.

In other embodiments, the invention further provides a kit comprising in a first container, a uPAR-binding molecule, and in a second container, a drug, wherein upon conjugation of the uPAR-binding molecule and the drug, the resulting conjugate has a rate of accumulation in a uPAR-expressing cell that is at least 20 to 40, 40 to 60, 60 to 80, 80 to 100, 100-200, 200-500, 500 to 1,000, 1,000 to 2,000, 2,000 to 2,500 fold greater than the rate of accumulation of an unconjugated form of the drug in the uPAR-expressing cell, and wherein the rates of accumulation of the conjugate and of the unconjugated form of the drug are measured by a method comprising: (a) culturing a population of the uPAR-expressing cell with the conjugate; (b) culturing a population of the uPAR-expressing cell with the unconjugated drug, wherein the populations of steps (a) and (b) are cultured under the same conditions; and (c) measuring the amount of the conjugate and the unconjugated drug accumulated in the populations of steps (a) and (b), respectively.

In other embodiments, the invention further provides a kit comprising a uPAR-binding molecule-drug conjugate in a container, wherein the conjugate has a rate of accumulation in a uPAR-expressing cell that is at least 20 to 40, 40 to 60, 60 to 80, 80 to 100, 100-200, 200-500, 500 to 1,000, 1,000 to 2,000, 2,000 to 2,500 fold greater than the rate of accumulation of an unconjugated form of the drug in the uPAR-expressing cell.

### 4. BRIEF DESCRIPTION OF THE FIGURES

FIG. 1. Amino acid sequence of receptor binding domain of human uPA residues 7-33 (Appella et al., 1987, J. Biol. Chem. 262(10):4437-4440).

FIGS. 2A & B. Amino acid sequence of receptor binding domain of human uPA: (A) residues 12-32 (SEQ ID NO:2); (B) residues 12-33 (SEQ ID NO:3) (Appella et al., 1987, J. Biol. Chem. 262(10):4437-4440).

FIG. 3. Effect of anti-human uPAR IgG (Mab 3936) on rat breast cancer cell line generated Mat B-III tumor volume. From day 10 to 20 post tumor cell inoculation, animals received control serum (Ctl), pre-immune rabbit IgG (Pre-immune), rabbit anti-rat uPAR IgG (RuPAR IgG), or mouse anti-human uPAR IgG (Mab 3936) (HuPAR IgG).

FIG. 4. Effect of anti-human uPAR IgG (Mab 3936) on Mat B-III tumor volume. From day 12 to Day 22 post tumor cell inoculation, animals received control serum, preimmune rabbit IgG, rabbit anti-rat uPAR IgG (RuPAR IgG), monoclonal uPAR antibodies No. 3F10 and R3, and two different dosages of mouse anti-human uPAR IgG (Mab 3936 at 100 μg/mL and 20 μg/animal).

FIG. 5. Preliminary dose defining study. Effect of anti-human uPAR IgG (Mab 3936), doxorubicin, Mab 3936-Doxorubicin conjugate on xenograft human breast cancer tumor model in nude mice (MDA-231 GFP tumors). Tumor volume (mm³) from week 8 to week 11 post tumor cell inoculation were compared. Bar graph from left to right: animals received control serum, doxorubicin via intravenous (DOX I.V.), doxorubicin via intraparenteral (DOX I.P.), Mab 3936 (3936) or Mab 3936-doxorubicin conjugate (3936+DOX) (2 mg/animal).

FIG. 6. Effect of anti-human uPAR IgG (Mab 3936), doxorubicin, Mab 3936-doxorubicin conjugate on xenograft human breast cancer tumor model in nude mice (MDA-231 GFP tumors). Tumor volume (mm³) from week 5 to week 15 post tumor cell inoculation were compared. Bar graph from left to right: animals received control serum, doxorubicin via intravenous, Mab 3936, doxorubicin via intraparenteral, and Mab 3936-doxorubicin conjugate.

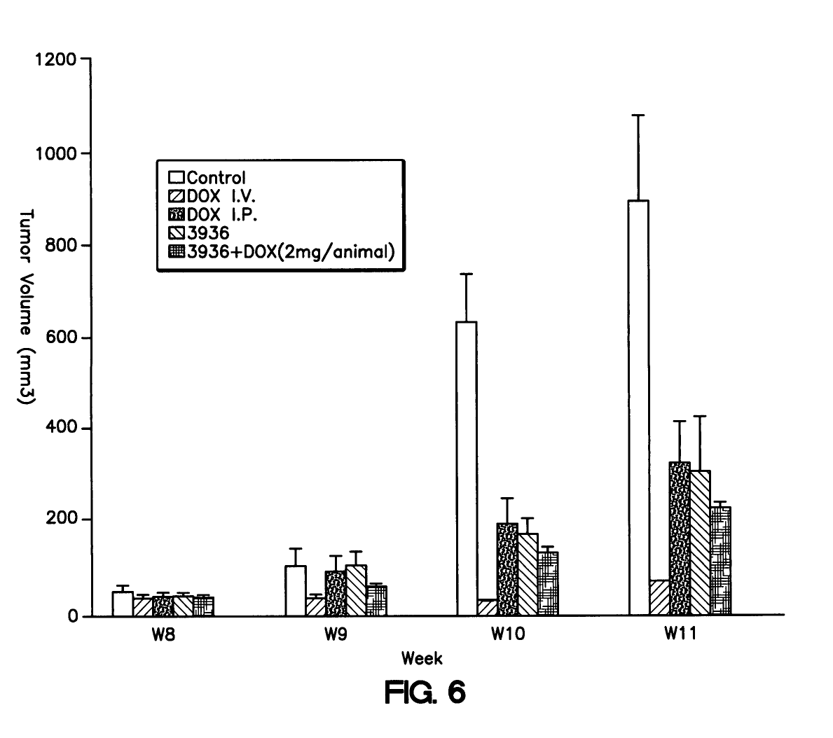

FIG. 7. Effect of anti-human uPAR IgG (Mab 3936), doxorubicin, Mab 3936-Doxorubicin conjugate (2 mg/animal), PAI-1 and PAI-1-Doxorubicin conjugate (PAI-1+DOX) (0.5 mg/animal) on MDA-231 GFP tumors. Tumor volume (mm³) from week 8 to week 13 post tumor cell inoculation were compared. Bar graph from left to right: animals received control serum, doxorubicin via intravenous (DOX I.V.), doxorubicin intraparenteral (DOX I.P.), Mab 3936, Mab 3936-doxorubicin conjugate (2 mg/animal), PAI-1, and PAI-1-doxorubicin conjugate (0.5 mg/animal).

FIG. 8. Effect of Mab 3936, doxorubicin, Mab 3936-Doxorubicin conjugate on MDA-MB-231 GFP tumors. Tumor volume (mm³) from week 8 to 13 post tumor cell inoculation were compared. Bar graph from left to right: animals received control serum, doxorubicin via intraparenteral, doxorubicin via intravenous, Mab 3936, and Mab 3936-doxorubicin conjugate.

FIG. 9. Effect of doxorubicin via intravenous or intraparenteral, PAI-1, PAI-1-Doxorubicin conjugate on MDA-MB-231 GFP tumors. Tumor volume (mm³) from week 8 to 13 post tumor cell inoculation were compared. Bar graph from left to right: animals received control serum, doxorubicin via intraparenteral, doxorubicin via intravenous, PAI-1, and PAI-1-doxorubicin conjugate.

### 4.1 Definitions

As used herein, the terms “antibody” and “antibodies” refer to polyclonal antibodies, monoclonal antibodies, multispecific antibodies, human antibodies, humanized antibodies, chimeric antibodies, single-chain Fvs (scFv), single chain antibodies, Fab fragments, F(ab′) fragments, disulfide-linked Fvs (sdFv), and anti-idiotypic (anti-Id) antibodies (including, e.g., anti-Id antibodies to antibodies of the invention), and epitope-binding fragments of any of the above. In particular, antibodies include immunoglobulin molecules and immunologically active fragments of immunoglobulin molecules, i.e., molecules that contain an antigen binding site. Immunoglobulin molecules can be of any type (e.g., IgG, IgE, IgM, IgD, IgA and IgY), class (e.g., IgG1, IgG2, IgG3, IgG4, IgA1 and IgA2) or subclass.

As used herein, the term “cancer” refers to a disease involving cells that have the potential to metastasize to distal sites and exhibit phenotypic traits that differ from those of non-cancer cells, for example, formation of colonies in a three-dimensional substrate such as soft agar or the formation of tubular networks or weblike matrices in a three-dimensional basement membrane or extracellular matrix preparation. Non-cancer cells do not form colonies in soft agar and form distinct sphere-like structures in three-dimensional basement membrane or extracellular matrix preparations. Cancer cells acquire a characteristic set of functional capabilities during their development, albeit through various mechanisms. Such capabilities include evading apoptosis, self-sufficiency in growth signals, insensitivity to anti-growth signals, tissue invasion/metastasis, limitless replicative potential, and sustained angiogenesis. The term “cancer cell” is meant to encompass both pre-malignant and malignant cancer cells.

As used herein, the term “metastasis” or “metastases” refer to the spread of cancer from its primary site to other places in the body. The term refer to a condition when cancer cells break away from a primary tumor, penetrate into lymphatic system and blood vessels, circulate through the bloodstream, and grow in a distant focus (metastasize) in normal tissues elsewhere in the body. Such metastases can include, but are not limited to, micrometastases.

As used herein, the term “derivative” in the context of proteins, polypeptides, peptides, and antibodies refers to proteins, polypeptides, peptides, and antibodies that comprise an amino acid sequence which has been altered by the introduction of amino acid residue substitutions, deletions, and/or additions. The term “derivative” as used herein also refers to proteins, polypeptides, peptides, and antibodies which have been modified, i.e., by the covalent attachment of any type of molecule to the proteins, polypeptides, peptides, and antibodies. For example, but not by way of limitation, proteins, polypeptides, peptides, and antibodies may be modified, e.g., by glycosylation, acetylation, pegylation, phosphorylation, amidation, derivatization by known protecting/blocking groups, proteolytic cleavage, linkage to a cellular ligand or other protein, etc. A derivative of proteins, polypeptides, peptides, and antibodies may be produced by chemical modifications using techniques known to those of skill in the art, including, but not limited to specific chemical cleavage, acetylation, formylation, metabolic synthesis of tunicamycin, etc. Further, a derivative of proteins, polypeptides, peptides, and antibodies may contain one or more non-classical amino acids. A derivative of proteins, polypeptides, peptides, and antibodies possess a similar or identical function as the proteins, polypeptides, peptides, and antibodies from which they were derived.

As used herein, the term “analog” in the context of proteins, polypeptides, peptides, and antibodies refers to proteins, polypeptides, peptides, and antibodies that are structurally analogous and/or functionally analogous to the proteins, polypeptides, peptides, and antibodies.

As used herein, the term “diagnosis” refers to a process of determining if an individual is afflicted with cancer or for determining the grade or stage of cancer. In this context, “diagnosis” refers to a process whereby one increases the likelihood that an individual is properly characterized as being afflicted with a cancer or a grade or stage of cancer or is properly characterized as not being afflicted with cancer or a grade or stage of cancer while minimizing the likelihood that the individual is improperly characterized as being afflicted with cancer or a grade or stage or cancer or improperly characterized as not being afflicted with cancer or a grade or stage of cancer.

As used herein, the term “effective amount” in the context of administering a therapy refers to the amount of a compound which is sufficient to reduce or ameliorate the progression, severity and/or duration of cancer or one or more symptoms thereof, prevent the development, recurrence or onset of cancer or one or more symptoms thereof, prevent the advancement or spread of cancer or one or more symptoms thereof, or enhance or improve the prophylacetic or therapeutic effect(s) of another therapy. In other embodiments, the term “effective amount” in the context of diagnosis is the amount of a compound which is sufficient to detect a gene product. For example, an effective amount of an antibody is that amount of an antibody sufficient to immunospecifically bind to and detect a protein of interest in a tissue or cell of interest.

As used herein, a “therapeutically effective amount” refers to that amount of the therapeutic agent sufficient to destroy, modify, control or remove primary, regional or metastatic cancer tissue. A therapeutically effective amount may refer to the amount of therapeutic agent sufficient to delay or minimize the spread of cancer or metastasis. A therapeutically effective amount may also refer to the amount of the therapeutic agent that provides a therapeutic benefit in the treatment or management of cancer. Further, a therapeutically effective amount with respect to a therapeutic agent of the invention means that amount of therapeutic agent alone, or in combination with other therapies, that provides a therapeutic benefit in the treatment or management of cancer. Used in connection with an amount of a therapeutic agent of the invention, the term can encompass an amount that improves overall therapy, reduces or avoids unwanted effects, or enhances the therapeutic efficacy of or synergies with another therapeutic agent. Preferably, a therapeutically effective amount of a therapy (e.g., a therapeutic agent) reduces the progression of cancer by at least 5%, preferably at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% relative to a control such as phosphate buffered saline (“PBS”).

The term “epitopes” as used herein refers to a fragment of a protein having antigenic or immunogenic activity in an animal, preferably in a mammal, and most preferably in a mouse or a human. An epitope having immunogenic activity is a fragment of a protein that elicits an antibody response in an animal. An epitope having antigenic activity is a fragment of a protein to which an antibody immunospecifically binds as determined by any method well known in the art, for example, by immunoassays. Antigenic epitopes need not necessarily be immunogenic.

As used herein, the term “fragment” or “portion” refers to a peptide or polypeptide comprising an amino acid sequence of at least 5 contiguous amino acid residues, at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least 80 contiguous amino acid residues, at least 90 contiguous amino acid residues, at least 100 contiguous amino acid residues, at least 125 contiguous amino acid residues, at least 150 contiguous amino acid residues, at least 175 contiguous amino acid residues, at least 200 contiguous amino acid residues, or at least 250 contiguous amino acid residues of the amino acid sequence of another polypeptide or a protein. In specific embodiments, a fragment or portion refers to a peptide or polypeptide comprising an amino acid sequence of 5-10, 11-15, 16-20, 21-25, 26-30, 31-35, 36-40, 41-45, 46-50, 51-55, 56-60, 61-65, 66-70, 71-75, 76-80, 81-85, 86-90, 91-95, 96-100, 101-105, 106-110, 111-115, 116-120, 121-150, 151-160, 161-170, 171-180, 181-190, 191-200, 201-220, 220-250 contiguous amino acid residues of the amino acid sequence of a polypeptide or protein. In a specific embodiment, a fragment of a protein or polypeptide retains at least one function of the protein or polypeptide. In another embodiment, a fragment of a protein or polypeptide retains at least two, three, four, or five functions of the protein or polypeptide. Preferably, a fragment of an antibody retains the ability to immunospecifically bind to an antigen.

As used herein, the term “functional fragment” refers to a peptide or polypeptide comprising an amino acid sequence of at least 5 contiguous amino acid residues, at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least 80 contiguous amino acid residues, at least 90 contiguous amino acid residues, at least 100 contiguous amino acid residues, at least 125 contiguous amino acid residues, at least 150 contiguous amino acid residues, at least 175 contiguous amino acid residues, at least 200 contiguous amino acid residues, or at least 250 contiguous amino acid residues of the amino acid sequence of second, different polypeptide, wherein said peptide or polypeptide retains at least one function of the second, different polypeptide.

As used herein, the term “fusion protein” refers to a polypeptide that comprises an amino acid sequence of a first protein or polypeptide or functional fragment, analog or derivative thereof, and an amino acid sequence of a heterologous protein, polypeptide, or peptide (i.e., a second protein or polypeptide or fragment, analog or derivative thereof different than the first protein or fragment, analog or derivative thereof). In one embodiment, a fusion protein comprises a prophylacetic or therapeutic agent fused to a heterologous protein, polypeptide or peptide. In accordance with this embodiment, the heterologous protein, polypeptide or peptide may or may not be a different type of prophylacetic or therapeutic agent.

As used herein, the term “immunospecifically binds to an antigen” and analogous terms refer to peptides, polypeptides, proteins, fusion proteins and antibodies or fragments thereof that specifically bind to an antigen and do not specifically bind to other antigens. A peptide, polypeptide, protein, or antibody that immunospecifically binds to an antigen may bind to other peptides, polypeptides, or proteins with lower affinity as determined by, e.g., immunoassays, or other assays known in the art. For example, antibodies or fragments that immunospecifically bind to an antigen may cross-reactive with related antigens. Preferably, antibodies or antibody fragments that immunospecifically bind to an antigen do not cross-react with other antigens.

As used herein, the term “in combination” refers to the use of more than one therapy (e.g., prophylacetic and/or therapeutic agents). The use of the term “in combination” does not restrict the order in which therapies (e.g., prophylacetic and/or therapeutic agents) are administered to a subject with cancer. A first therapy (e.g., a prophylacetic or therapeutic agent) can be administered prior to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy (e.g., a prophylacetic or therapeutic agent) to a subject which had, has, or is susceptible to cancer. The therapies (e.g., prophylacetic or therapeutic agents) are administered to a subject in a sequence and within a time interval such that the therapy of the invention can act together with the other therapy to provide an increased benefit than if they were administered otherwise. Any additional therapy (e.g., prophylacetic or therapeutic agent) can be administered in any order with the other additional therapies (e.g., prophylacetic or therapeutic agents).

As used herein, the term “isolated” in the context of a peptide, polypeptide, fusion protein, antibody or conjugate refers to a peptide, polypeptide, fusion protein, antibody or conjugate which is substantially free of cellular material or contaminating proteins from the cell or tissue source from which it is derived, or substantially free of chemical precursors or other chemicals when chemically synthesized. The language “substantially free of cellular material” includes preparations of a peptide, polypeptide, fusion protein, antibody or conjugate in which the peptide, polypeptide, fusion protein, antibody or conjugate is separated from cellular components of the cells from which it is isolated or recombinantly produced. Thus, a peptide, polypeptide, fusion protein, antibody or conjugate that is substantially free of cellular material includes preparations of a peptide, polypeptide, fusion protein, antibody or conjugate having less than about 30%, 20%, 10%, or 5% (by dry weight) of heterologous peptide, polypeptide, fusion protein, antibody or conjugate (also referred to herein as a “contaminating protein”). When the peptide, polypeptide, fusion protein, antibody or conjugate is recombinantly produced, it is also preferably substantially free of culture medium, i.e., culture medium represents less than about 20%, 10%, or 5% of the volume of the protein preparation. When the peptide, polypeptide, fusion protein, antibody or conjugate is produced by chemical synthesis, it is preferably substantially free of chemical precursors or other chemicals, i.e., it is separated from chemical precursors or other chemicals which are involved in the synthesis of the peptide, polypeptide, fusion protein, antibody or conjugate. Accordingly such preparations of a peptide, polypeptide, fusion protein, antibody or conjugate have less than about 30%, 20%, 10%, 5% (by dry weight) of chemical precursors or compounds other than the peptide, polypeptide, fusion protein, antibody or conjugate of interest.

As used herein, the phrase “non-responsive/refractory” is used to describe patients treated with one or more currently available therapies (e.g., cancer therapies) such as chemotherapy, radiation therapy, surgery, hormonal therapy and/or biological therapy/immunotherapy, particularly a standard therapeutic regimen for the particular cancer, wherein the therapy is not clinically adequate to treat the patients such that these patients need additional effective therapy, e.g., remain unsusceptible to therapy. The phrase can also describe patients who respond to therapy yet suffer from side effects, relapse, develop resistance, etc. In various embodiments, “non-responsive/refractory” means that at least some significant portion of the cancer cells are not killed or their cell division arrested. The determination of whether the cancer cells are “non-responsive/refractory” can be made either in vivo or in vitro by any method known in the art for assaying the effectiveness of treatment on cancer cells, using the art-accepted meanings of “refractory” in such a context. In various embodiments, a cancer is “non-responsive/refractory” where the number of cancer cells has not been significantly reduced, or has increased during the receipt of the therapy.

As used herein, the phrase “pharmaceutically acceptable salt(s),” includes, but is not limited to, salts of acidic or basic groups. The acids that can be used to prepare pharmaceutically acceptable salts are those that form non-toxic salts, i.e., salts containing pharmacologically acceptable anions, including but not limited to sulfuric, citric, maleic, acetic, oxalic, hydrochloride, hydrobromide, hydroiodide, nitrate, sulfate, bisulfate, phosphate, acid phosphate, isonicotinate, acetate, lactate, salicylate, citrate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Compounds that include an amino moiety may form pharmaceutically acceptable salts with various amino acids, in addition to the acids mentioned above. Compounds that are acidic in nature are capable of forming base salts with various pharmacologically acceptable cations. Examples of such salts include alkali metal or alkaline earth metal salts and, particularly, calcium, magnesium, sodium lithium, zinc, potassium, and iron salts.

As used herein, the term “population” in context of subjects refers to 2 or more, preferably 5 or more, 10 or more, 25 or more, 50 or more, 100 or more, 150 or more, 200 or more, 250 or more, 300 or more, or 500 or more subjects.

As used herein, the terms “prevent,” “preventing” and “prevention” refer to the prevention of the development, recurrence, onset or spread of cancer or one or more symptoms thereof resulting from the administration of one or more conjugates of the invention or the administration of a combination of such a conjugate and another therapy.

As used herein, the terms “manage,” “managing” and “management” refer to the beneficial effects that a subject derives from a therapy (e.g., a prophylacetic or therapeutic agent) which does not result in a cure of cancer. In certain embodiments, a subject is administered one or more therapies to “manage” cancer so as to prevent the progression or worsening of the cancer.

As used herein, the terms “treat,” “treating” and “treatment” refer to the eradication, reduction or amelioration of cancer or a symptom thereof, particularly, the eradication, removal, modification, or control of primary, regional, or metastatic cancer tissue that results from the administration of one or more therapies. In certain embodiments, such terms refer to the minimizing or delaying the spread of cancer resulting from the administration of one or more therapies to a subject with cancer.

As used herein, the term “prophylacetic agent” refers to any compound(s) which can be used in the prevention of cancer. In certain embodiments, the term “prophylacetic agent” refers to a conjugate of the present invention. In certain other embodiments, the term “prophylacetic agent” refers to an agent other than a compound identified in the screening assays described herein which is known to be useful for, or has been or is currently being used to prevent or impede the onset, development and/or progression of cancer or one or more symptoms thereof.

As used herein, the phrase “side effects” encompasses unwanted and adverse effects of a prophylacetic or therapeutic agent. Adverse effects are always unwanted, but unwanted effects are not necessarily adverse. An adverse effect from a prophylacetic or therapeutic agent might be harmful or uncomfortable or risky. Side effects from chemotherapy include, but are not limited to, gastrointestinal toxicity such as, but not limited to, early and late-forming diarrhea and flatulence, nausea, vomiting, anorexia, leukopenia, anemia, neutropenia, asthenia, abdominal cramping, fever, pain, loss of body weight, dehydration, alopecia, dyspnea, insomnia, dizziness, mucositis, xerostomia, and kidney failure, as well as constipation, nerve and muscle effects, temporary or permanent damage to kidneys and bladder, flu-like symptoms, fluid retention, and temporary or permanent infertility. Side effects from radiation therapy include but are not limited to fatigue, dry mouth, and loss of appetite. Side effects from biological therapies/immunotherapies include but are not limited to rashes or swellings at the site of administration, flu-like symptoms such as fever, chills and fatigue, digestive tract problems and allergic reactions. Side effects from hormonal therapies include but are not limited to nausea, fertility problems, depression, loss of appetite, eye problems, headache, and weight fluctuation. Additional undesired effects typically experienced by patients are numerous and known in the art. Many are described in the Physicians' Desk Reference (59th ed., 2005).

As used herein, the terms “subject” and “patient” are used interchangeably to refer to an animal (e.g., a mammal, a fish, an amphibian, a reptile, a bird and an insect). In a specific embodiment, a subject is a mammal (e.g., a non-human mammal and a human). In another embodiment, a subject is a pet (e.g., a dog, a cat, a guinea pig, a monkey and a bird), a farm animal (e.g., a horse, a cow, a pig, a goat and a chicken) or a laboratory animal (e.g., a mouse and a rat). In another embodiment, a subject is a primate (e.g., a chimpanzee and a human). In a preferred embodiment, a subject is a human.

As used herein, the term “synergistic” refers to a combination of a therapy described herein, and another therapy (e.g., agent), which is more effective than the additive effects of the therapies. Preferably, such other therapy has been or is currently being to prevent, treat, manage or ameliorate cancer or a symptom thereof. A synergistic effect of a combination of therapies (e.g., prophylacetic or therapeutic agents) permits the use of lower dosages of one or more of the therapies and/or less frequent administration of said therapies to a subject with cancer. The ability to utilize lower dosages of a therapy (e.g., a prophylacetic or therapeutic agent) and/or to administer said therapy less frequently reduces the toxicity associated with the administration of said agent to a subject without reducing the efficacy of said therapies in the prevention, treatment, management or amelioration of cancer. In addition, a synergistic effect can result in improved efficacy of therapies (e.g., agents) in the prevention, treatment, management or amelioration of cancer. Finally, a synergistic effect of a combination of therapies (e.g., prophylacetic or therapeutic agents) may avoid or reduce adverse or unwanted side effects associated with the use of either therapy alone.

As used herein, the terms “chemotherapeutic agent” and “chemotherapeutic agents” refer to selectively toxic substances that inhibit the growth of cancer tissue but are less inhibitory to the growth of normal cells. Chemotherapeutic agents are more toxic to rapidly proliferating cells such as those associated with cancer than to normal cells.

As used herein, the terms “therapeutic agent(s)” refers to any substances that can be used in the treatment, management or amelioration of cancer, metastasis or one or more symptoms thereof.

### 5. DETAILED DESCRIPTION OF THE INVENTION

The present inventors have identified an effective system for delivery of drug using uPAR-binding molecule-drug conjugates of the present invention. In preferred embodiments, the drug is a chemotherapeutic agent.

Accordingly, the present invention provides uPAR-binding molecule-drug conjugate comprising a uPAR-binding portion conjugated to a drug, which is capable of accumulation in uPAR-expressing cells. The uPAR-binding portion of the invention is preferably conjugated to the drug of the uPAR-binding molecule-drug conjugate via a linker, most preferably a linker that is hydrolyzed upon uptake of the conjugate into a uPAR-expressing cell. In a specific embodiment, the uPAR-binding portion of the conjugates of the present invention is conjugated to a drug directly without a linker. The present invention yet further provides methods of treatment of metastases involving uPAR-expressing cells comprising administering to a patient in need of such treatment a uPAR-binding molecule-drug conjugate of the invention, in either single therapy or combination therapy regimens. The present invention further provides pharmaceutical compositions and kits comprising such conjugates.

The uPAR-binding molecule-drug conjugate may be used in the detection of uPAR in a patient sample. The uPAR-binding molecule-drug conjugate may also be used, for example, in the detection of uPAR in vivo and may, therefore, be utilized as part of a detection, diagnosis, and in vivo imaging of primary tumors and of, metastases, preferably micrometastases in a subject, by introducing a labeled uPAR-binding molecule-drug conjugate to a subject. After a time sufficient to allow for distribution and accumulation in vivo, direct imaging may be performed during the treatment process. A variety of methods can be used to detect accumulated labeled material in vivo, including but not limited to radioimaging techniques, e.g., X-ray, CAT scan, and magnetic resonance imaging (MRI), sonography, and positron emission tomography (PET).

### 5.1 uPAR-Binding Molecule-Drug Conjugates

Described herein is a uPAR-binding molecule-drug conjugate comprising a first portion which immunospecifically binds human uPAR and a second portion which comprises a drug, wherein the conjugate is capable of being internalized into a uPAR-expressing cell.

5.1.1 Peptides, Derivatives and Analogs Thereof

In an embodiment of the invention, uPAR binding molecules that are useful for making the uPAR-binding molecule-drug conjugate of the present invention include peptides, derivatives and analogs thereof. In specific embodiments, the peptide is a peptide mimetic. In specific embodiments, the peptide mimetic mimics the structure of a fragment of the uPA protein. In specific embodiments, the peptide mimetic mimics the function of a fragment of the uPA protein. In one specific embodiment, peptide libraries can be screened to select a peptide with the desired activity; such screening can be carried out by assaying, e.g., for binding to uPAR. In a preferred embodiment, the uPAR-binding molecule portion of the uPAR-binding molecule-drug conjugate is a CDR of an anti-uPAR antibody. In specific embodiments, the uPAR-binding molecule portion of the uPAR-binding molecule-drug conjugate is CDR1, CDR2, CDR3 of the light chain. In specific embodiments, the uPAR-binding molecule portion of the uPAR-binding molecule-drug conjugate is CDR1, CDR2, CDR3 of the heavy chain. In a preferred embodiment, the uPAR-binding molecule portion of the uPAR-binding molecule-drug conjugate is a CDR of the anti-uPAR antibody 3936. In a preferred embodiment, the uPAR-binding molecule portion of the uPAR-binding molecule-drug conjugate is capable of binding to uPAR and are internalized into the uPAR-expressing cells.

In vitro systems may be designed to identify molecules capable of binding to uPAR. These uPAR-binding molecules are useful for making the uPAR-binding molecules-drug conjugate of the present invention. The principle of the assays used to identify molecules that binds to uPAR involves preparing a reaction mixture of the uPAR, or fragments thereof and the test molecules under conditions and for a time sufficient to allow the two components to bind, thus forming a complex, which can represent a transient complex, which can be removed and/or detected in the reaction mixture. These assays can be conducted in a variety of ways. For example, one method to conduct such an assay would involve anchoring uPAR or the test molecule onto a solid phase and detecting uPAR/test molecule complexes anchored on the solid phase at the end of the reaction. In one embodiment of such a method, the uPAR or fragment thereof may be anchored onto a solid surface, and the test molecule, which is not anchored, may be labeled, either directly or indirectly.

In practice, microtitre plates may conveniently be utilized as the solid phase. The anchored component may be immobilized by non-covalent or covalent attachments. Non-covalent attachment may be accomplished by simply coating the solid surface with a solution of the uPAR and drying. Alternatively, an immobilized antibody, preferably a monoclonal antibody, specific for uPAR may be used to anchor the protein to the solid surface. In order to conduct the assay, the nonimmobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously nonimmobilized component is pre-labeled, the detection of label immobilized on the surface indicates that complexes were formed. Alternatively, a reaction can be conducted in a liquid phase, the reaction products separated from unreacted components, and complexes detected, e.g., using an immobilized antibody specific for uPAR to anchor any complexes formed in solution.

In preferred embodiments, the peptides or peptide mimetics are selected to mimic the receptor binding domain of uPA. The peptides or peptide mimetics of uPA binds to uPAR and are internalized into the uPAR-expressing cells. In certain embodiments, the peptides or peptide mimetics mimic a portion or the entire the amino-terminal A chain of uPAR (amino acid residues 1-158 of SEQ ID NO:5). In specific embodiments, the peptides or peptide mimetics mimic the growth factor domain (amino acid residues 1-49 of SEQ ID NO:5), the kringle domain (amino acid residues 50-131 of SEQ ID NO:5), the linker region (amino acid residues 132-158 of SEQ ID NO:5). In certain embodiments, the peptides or peptide mimetics mimic a portion or the entire B chain (amino acid residues 159-411 of SEQ ID NO:5). In certain embodiments, the peptides or peptide mimetics do not mimic the growth factor domain, the kringle domain, the linker or the B chain of uPA.

In preferred embodiments, the peptides or peptide mimetics mimic the 15 kD amino-terminal fragment (ATF of the uPA molecule SEQ ID NO:4). In specific embodiments, the peptides or peptide mimetics mimic residues 1-135, 1-143, 1-164 of SEQ ID NO: 5. In specific embodiments, the peptides or peptide mimetics mimic the cysteine-rich region known as the growth factor region. In more specific embodiments, the peptides or peptide mimetics mimic residues 7-33 of uPA (SEQ ID NO:1), residues 12-32 of the uPA (SEQ ID NO:2) or residues 12-33 of uPA (SEQ ID NO:3). In more specific embodiments, the peptides or peptide mimetics mimic a portion of 3, 4, 5, 6, 7, 8, 9, 10, 11, 12, 13, 14, 15, 16, 17, 18, 19, 20, 21 consecutive residues of SEQ ID NO:3. In more specific embodiments, the peptides or peptide mimetics mimic a portion of 3, 4, 5, 6, 7, 8, 9, 10, 11, 12, 13, 14, 15, 16, 17, 18, 19, 20, 21 consecutive residues of SEQ ID NO:4. In other specific embodiments, the uPAR-binding molecule comprises more than one peptides or peptide mimetics wherein each peptide or peptide mimetic mimics a portion of the uPA of SEQ ID NO:4. In other specific embodiments, the uPAR-binding molecule comprises more than one peptides or peptide mimetics wherein each peptide or peptide mimetic mimics a portion of the uPA of SEQ ID NO:5.

In preferred embodiments, the peptides or peptide mimetics comprises the ATF, or growth factor region of uPA. In specific embodiments, the peptides or peptide mimetics comprise residues 1-135, 1-143, 1-164 of SEQ ID NO:5. In more specific embodiments, the peptides or peptide mimetics comprise residues 7-33 of uPA (SEQ ID NO:1), residues 12-32 of the uPA (SEQ ID NO:2) or residues 12-33 of uPA (SEQ ID NO:3). In more specific embodiments, the peptides or peptide mimetics comprise a portion of 3, 4, 5, 6, 7, 8, 9, 10, 11, 12, 13, 14, 15, 16, 17, 18, 19, 20, 21 consecutive residues of SEQ ID NO:3. In more specific embodiments, the peptides or peptide mimetics comprise a portion of 3, 4, 5, 6, 7, 8, 9, 10, 11, 12, 13, 14, 15, 16, 17, 18, 19, 20, 21 consecutive residues of SEQ ID NO:4 or 5. In other specific embodiments, the uPAR-binding molecule comprises more than one peptides or peptide mimetics wherein each peptide or peptide mimetic comprises a portion of the uPA of SEQ ID NO:3. In other specific embodiments, the uPAR-binding molecule comprises more than one peptides or peptide mimetics wherein each peptide or peptide mimetic comprises a portion of the uPA of SEQ ID NO:4 or 5.

In particular embodiments of the invention, the peptides or peptide mimetics are selected to mimic the following sequences of human uPA:

| | |
|---|---|
| VPSNCDCLNGGTCVSNKYFSNIHWCNC; | (SEQ ID NO:1) |
| DCLNGGTCVSNKYFSNIHWCN; | (SEQ ID NO:2) |
| and | |
| DCLNGGTCVSNKYFSNIHWCNC. | (SEQ ID NO:3) |

In particular embodiments of the invention, the peptides or peptide mimetics comprise the following sequences of human uPA:

| | |
|---|---|
| VPSNCDCLNGGTCVSNKYFSNIHWCNC; | (SEQ ID NO:1) |
| DCLNGGTCVSNKYFSNIHWCN; | (SEQ ID NO:2) |
| and | |
| DCLNGGTCVSNKYFSNIHWCNC. | (SEQ ID NO:3) |

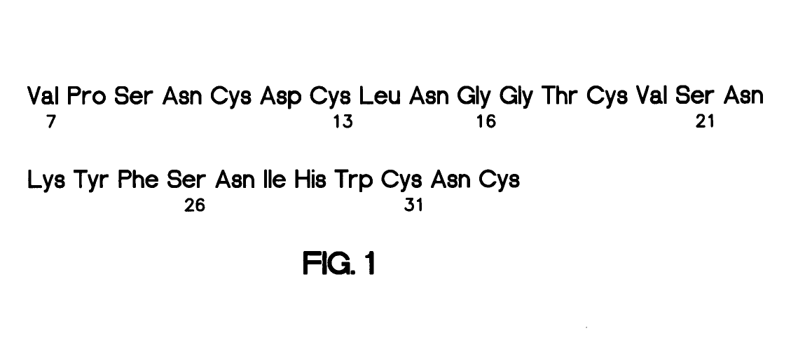

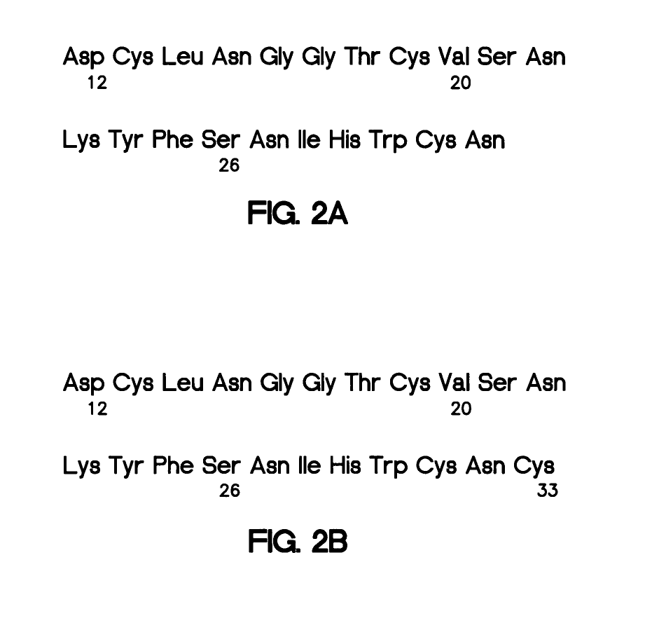

In a specific embodiment, uPA derivatives and analogs, in particular uPA fragments and derivatives of such fragments, that comprise one or more domains of a uPA protein may be used to make the uPAR-binding molecule of the uPAR-binding molecule-drug conjugate. In specific embodiments, the uPAR-binding molecule is a peptide or derivative thereof that binds uPAR, for example, but not limited to, the peptides having the amino acid sequence of SEQ ID NO:1 (FIG. 1) and SEQ ID NO:2 (FIG. 2). In certain embodiments, the uPAR-binding molecule-drug conjugate do not include uPA, uPA derivatives and analogs. In other embodiments, the uPAR-binding molecule-drug conjugate contains an about 4 to 10, 10-20, 20-40, 40-60, 60-80, 80-100, 100-150, 150-200, 200-250, 250-300, 300-350, 350-400, 400-431 consecutive amino acid sequence of human uPA, GenBank accession no. CAA01390, SEQ ID NO:5. In other embodiments, the uPAR-binding molecule comprises two, three, four, five, six, seven, eight, nine, ten separate peptide sequences having an about 3-10, 10-20, 20-40, 40-60, 60-80, 80-100, 100-150, 150-200, 200-250, 250-300, 300-350, 350-400, 400-431 consecutive amino acid sequence of human uPA.

In another specific embodiment, the invention uses a uPA protein, fragment, analog, or derivative which is expressed as a fusion, or chimeric protein product (comprising the protein, fragment, analog, or derivative joined via a peptide bond to a heterologous protein sequence (of a different protein)). A specific embodiment relates to a chimeric protein comprising a fragment of uPA of about 6-8, 8-10, 10-12, 12-14, 14-16, 16-20, 20-25, 25-30, 30-35 consecutive amino acids of uPA, preferably the uPA fragment is the amino terminal fragment, growth factor domain, kringle domain, linker region, peptides comprising the amino acid sequence of SEQ ID NO:1, SEQ ID NO:2, SEQ ID NO:3.

Peptides, derivatives and analogs thereof, and peptide mimetics that specifically bind uPAR can be produced by various methods known in the art, including, but not limited to solid-phase synthesis or by solution (Nakanishi et al., 1993, Gene 137:51-56; Merrifield, 1963, J. Am. Chem. Soc. 15:2149-2154; Neurath, H. et al., Eds., The Proteins, Vol II, 3d Ed., p. 105-237, Academic Press, New York, N.Y. (1976). For example, a peptide that is capable of binding to uPAR or a peptide that is corresponding to a portion of a uPA protein which comprises the desired domain or binding to a receptor, can be synthesized by use of a peptide synthesizer. Alternatively, uPA derivatives can be made by altering uPA sequences by substitutions, additions or deletions that provide for functionally equivalent molecules. The uPA derivatives that are useful for the present invention include, but are not limited to, those containing, as a primary amino acid sequence, all or part of the amino acid sequence of a uPA peptide including altered sequences in which functionally equivalent amino acid residues are substituted for residues within the sequence resulting in a silent change. For example, one or more amino acid residues within the sequence can be substituted by another amino acid of a similar polarity which acts as a functional equivalent, resulting in a silent alteration. Substitutes for an amino acid within the sequence may be selected from other members of the class to which the amino acid belongs. For example, the nonpolar (hydrophobic) amino acids include alanine, leucine, isoleucine, valine, proline, phenylalanine, tryptophan and methionine. The polar neutral amino acids include glycine, serine, threonine, cysteine, tyrosine, asparagine, and glutamine. The positively charged (basic) amino acids include arginine, lysine and histidine. The negatively charged (acidic) amino acids include aspartic acid and glutamic acid.

Furthermore, if desired, nonclassical amino acids or chemical amino acid analogs can be introduced as a substitution or addition into a uPA sequence. Non-classical amino acids include but are not limited to the D-isomers of the common amino acids, α-amino isobutyric acid, 4-aminobutyric acid, hydroxyproline, sarcosine, citrulline, cysteic acid, t-butylglycine, t-butylalanine, phenylglycine, cyclohexylalanine, α-alanine, designer amino acids such as β-methyl amino acids, Cα-methyl amino acids, and Nα-methyl amino acids.

Included within the scope of the invention are uPA protein fragments or other derivatives or analogs which are differentially modified during or after translation, e.g., by glycosylation, acetylation, phosphorylation, amidation, derivatization by known protecting/blocking groups, proteolytic cleavage, linkage to an antibody molecule or other cellular ligand, etc. Any of numerous chemical modifications may be carried out by known techniques, including but not limited to specific chemical cleavage by cyanogen bromide, trypsin, chymotrypsin, papain, V8 protease, NaBH4; acetylation, formylation, oxidation, reduction; metabolic synthesis in the presence of tunicamycin, etc.

In a specific embodiment, the uPAR-binding molecule is uPA, its derivative or analog that is functionally active, i.e., capable of exhibiting one or more functional activities associated with a full-length, wild-type uPA protein. Derivatives or analogs of uPA include but are not limited to those peptides which are substantially homologous to uPA or fragments thereof. To determine the percent identity of two amino acid sequences, e.g., between the amino acid sequences of uPA and its derivative or analog, the sequences are aligned for optimal comparison purposes (e.g., gaps can be introduced in the first amino acid sequence for optimal alignment with a second amino acid sequence). The amino acid residues at corresponding amino acid positions are then compared. When a position in the first sequence is occupied by the same amino acid residue as the corresponding position in the second sequence, then the molecules are identical at that position. The percent identity between the two sequences is a function of the number of identical positions shared by the sequences (i.e., % identity=# of identical positions/total # of positions (e.g., overlapping positions)×100). In one embodiment, the two sequences are the same length.

The determination of percent identity between two sequences can be accomplished using a mathematical algorithm. A preferred, non-limiting example of a mathematical algorithm utilized for the comparison of two sequences is the algorithm of Karlin and Altschul, 1990, Proc. Natl. Acad. Sci. USA 87:2264-2268, modified as in Karlin and Altschul, 1993, Proc. Natl. Acad. Sci. USA 90:5873-5877. Such an algorithm is incorporated into the NBLAST and XBLAST programs of Altschul, et al., 1990, J. Mol. Biol. 215:403-410. BLAST nucleotide searches can be performed with the NBLAST program, score=100, wordlength=12 to obtain amino acid sequences homologous to uPA. BLAST protein searches can be performed with the XBLAST program, score=50, wordlength=3 to obtain amino acid sequences homologous to uPA. To obtain gapped alignments for comparison purposes, Gapped BLAST can be utilized as described in Altschul et al., 1997, Nucleic Acids Res. 25:3389-3402. Alternatively, PSI-Blast can be used to perform an iterated search which detects distant relationships between molecules (id.). When utilizing BLAST, Gapped BLAST, and PSI-Blast programs, the default parameters of the respective programs (e.g., XBLAST and NBLAST) can be used. Another preferred, non-limiting example of a mathematical algorithm utilized for the comparison of sequences is the algorithm of Myers and Miller, CABIOS (1989). Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Additional algorithms for sequence analysis are known in the art and include ADVANCE and ADAM as described in Torellis and Robotti, 1994, Comput. Appl. Biosci. 10:3-5; and FASTA described in Pearson and Lipman, 1988, Proc. Natl. Acad. Sci. 85:2444-8. Within FASTA, ktup is a control option that sets the sensitivity and speed of the search. If ktup=2, similar regions in the two sequences being compared are found by looking at pairs of aligned residues; if ktup=1, single aligned amino acids are examined. ktup can be set to 2 or 1 for protein sequences, or from 1 to 6 for DNA sequences. The default if ktup is not specified is 2 for proteins and 6 for DNA. Alternatively, protein sequence alignment may be carried out using the CLUSTAL W algorithm, as described by Higgins et al., 1996, Methods Enzymol. 266:383-402.

The percent identity between two sequences can be determined using techniques similar to those described above, with or without allowing gaps. In calculating percent identity, only exact matches are counted over the length of the aligned sequences.

The peptides, derivatives and analogs thereof may be isolated and purified by standard methods including chromatography (e.g., ion exchange, affinity, and sizing column chromatography), centrifugation, differential solubility, or by any other standard technique for the purification of peptides. The functional properties may be evaluated using any suitable assay, including, but not limited to, competitive and non-competitive binding assays. Examples of binding assays are well known in the art.

In other embodiments, the uPAR-binding molecule is a non-peptide mimetic.

5.1.2 Anti-uPAR Antibodies

In a preferred embodiment, the uPAR-binding molecule useful for making the uPAR-binding molecule-drug conjugate of the present invention is an antibody. In certain embodiments, the antibody is directed against uPAR or a subsequence, analogue or variant thereof. In certain embodiments, the antibody is capable of binding to uPAR and is internalized into the uPAR-expressing cells. In certain embodiments, the anti-uPAR antibodies that are useful in the present invention immunospecifically binds human uPAR (SEQ ID NO: 7). In specific embodiments, the anti-uPAR antibodies may be raised against or directed substantially against a specific region of uPAR, i.e., an epitope. In preferred embodiments, the anti-uPAR antibodies bind the transmembrane domain of uPAR and are internalized into the uPAR-expressing cells. In other specific embodiments, the anti-uPAR antibodies specifically bind to glycosylated variants of uPAR. In other specific embodiments, the anti-uPAR antibodies specifically bind to a ligand binding domain of uPAR, hydrophilic N-terminal portion, uPA binding domain, binding domain for a ligand other than uPA, or non-binding portion of uPAR. In specific embodiments, the anti-uPAR antibodies specifically bind amino acid residues 1-20, 21-30, 31-40, 41-50, 51-60, 61-70, 71-80, 81-87, 88-95, 96-110, 111-120, 121-130, 130-150, 150-200, 201-220, 221-250, 251-270, 270-281, 282-299, 300-313 of SEQ ID NO:7. In a preferred embodiment, the anti-uPAR antibodies specifically bind the uPA binding domain or a portion of uPAR at amino acid residues 1-87 of SEQ ID NO:7.

In other specific embodiments, the anti-uPAR antibodies specifically bind an unbound uPAR. In other specific embodiments, the anti-uPAR antibodies specifically bind uPAR that is bound to a ligand. In other specific embodiments, the anti-uPAR antibodies specifically bind a uPAR-uPA complex. In other specific embodiments, the anti-uPAR antibodies specifically bind a complex comprising uPAR, uPA and other proteins.

Any human, humanized or chimeric anti-uPAR antibody can be employed in the methods and compositions of the invention. The anti-uPAR antibodies used in the present methods and compositions are preferably monoclonal, and may be multispecific, human, humanized or chimeric antibodies, single chain antibodies, Fab fragments, F(ab′) fragments, fragments produced by a Fab expression library, and uPAR binding fragments of any of the above. The term “antibody,” as used herein, refers to immunoglobulin molecules and immunologically active portions of immunoglobulin molecules, i.e., molecules that contain an antigen binding site that immunospecifically binds uPAR. The immunoglobulin molecules of the invention can be of any type (e.g., IgG, IgE, IgM, IgD, IgA and IgY), class (e.g., IgG1, IgG2, IgG3, IgG4, IgA1 and IgA2) or subclass of immunoglobulin molecule.

In certain embodiments of the invention, UPAR-human antigen-binding antibody fragments can be used in the present invention include, but are not limited to, Fab, Fab′ and F(ab′)₂, Fd, single-chain Fvs (scFv), single-chain antibodies, disulfide-linked Fvs (sdFv) and fragments comprising either a V_{L} or V_{H} domain. Antigen-binding antibody fragments, including single-chain antibodies, may comprise the uPAR-binding variable region(s) alone or in combination with the entirety or a portion of the following: hinge region, CH1, CH2, CH3 and CL domains. Also included in the invention are antigen-binding fragments also comprising any combination of variable region(s) with a hinge region, CH1, CH2, CH3 and CL domains. Preferably, the variable regions are derived human, murine (e.g., mouse and rat), donkey, sheep, rabbit, goat, guinea pig, camelid, horse, or chicken antibodies. As used herein, “human” antibodies include antibodies having the amino acid sequence of a human immunoglobulin and include antibodies isolated from human immunoglobulin libraries, from human B-cells, or from animals transgenic for one or more human immunoglobulin, as described infra and, for example in U.S. Pat. No. 5,939,598 by Kucherlapati et al.

The anti-uPAR antibodies that may be used in the methods of the present invention may be monospecific, bispecific, trispecific or of greater multispecificity. Multispecific antibodies may be specific for different epitopes of uPAR or may be specific for both uPAR as well as for a heterologous protein. See, e.g., PCT publications WO 93/17715; WO 92/08802; WO 91/00360; WO 92/05793; Tutt, et al., 1991, J. Immunol. 147:60-69; e.g., U.S. Pat. Nos. 4,474,893; 4,714,681; 4,925,648; 5,573,920; 5,601,819; Kostelny et al., 1992, J. Immunol. 148:1547-1553.

In a preferred embodiment, the uPAR-binding molecule of a uPAR-binding molecule-drug conjugate of the invention is monoclonal antibody 3936 or a monoclonal antibody, which immunospecifically bind to the epitope of uPAR which is recognized by the anti-human uPAR monoclonal antibody 3936. In other preferred embodiments, the uPAR-binding molecule of a uPAR-binding molecule-drug conjugate of the invention competes for binding or binds to the same epitope as monoclonal antibody 3936.

Antibodies that are useful in the present invention may be described or specified in terms of the particular variable regions or CDRs they comprise. In certain embodiments antibodies useful for the invention comprise one or more CDRs of the anti-uPAR antibody 3936. In a highly preferred embodiment, the anti-uPAR antibody comprises the heavy and/or light chain variable region or 1, 2, 3, 4, 5, or 6 CDRs of monoclonal antibody 3936. In specific embodiments, the anti-uPAR antibody is a humanized antibody with one or more CDRs from the monoclonal antibody 3936 and a human framework region. In certain embodiments, one or more CDRs of the anti-uPAR antibody have 1, 2, 3, 4, 5, 6, 7, 8, 9, or 10 amino acid substitutions. In other embodiments, anti-uPAR antibody have 1, 2, 3, 4, 5, 6, 7, 8, 9, or 10 amino acid substitutions in the framework region. In more preferred embodiments, the amino acid substitutions are conservative substitutions. In the preferred embodiment, the anti-uPAR antibody that is useful for the present invention immunospecifically bind to the epitope of uPAR which is recognized by the anti-human uPAR Mab 3936. In a most preferred embodiment, the uPAR-binding molecule is Mab 3936. In a preferred embodiment, those antibodies comprise human constant regions. In a most preferred embodiment, those antibodies comprise human constant and framework regions. Methods of generating such antibodies are described below.

Additionally, anti-uPAR antibodies for use in the methods and compositions of the present invention may also be described or specified in terms of their primary structures. Antibodies having regions of at least 50%, at least 55%, at least 60%, at least 65%, at least 70%, at least 75%, at least 80%, at least 85%, at least 90%, at least 95% and most preferably at least 98% identity (as calculated using methods known in the art and described herein in Section 5.1.1) to the CDRs or variable regions of Mab 3936 are also included in the present invention. In certain embodiments, Antibodies having regions of at most 50%, at most 55%, at most 60%, at most 65%, at most 70%, at most 75%, at most 80%, at most 85%, at most 90%, at most 95% or at most 98% identity (as calculated using methods known in the art and described herein in Section 5.1.1) to the CDRs or variable regions of Mab 3936 are also included in the present invention. In preferred embodiments, anti-uPAR antibodies useful for the present invention have 1, 2, 3, 4, 5, 6, 7, 8, 9, 10, 11-20, 21-30, 31-40, 41-50, 51-60, 61-70 amino acid substitutions at the CDRs or framework region of Mab 3936. In more preferred embodiments, the amino acid substitutions are conservative substitutions.

Anti-uPAR antibody that are useful for the uPAR-binding molecule-drug conjugate of the present invention has amino acid substitutions relative to a Mab 3936 that resulting in improved affinity for uPAR relative to the native antibody. In certain embodiments, such an antibody can be humanized. An exemplary method for identifying anti-uPAR antibodies with increased affinity is through systematic mutagenesis and screening, preferably reiterative screening, for antibodies with improved affinity to uPAR, for example as described by Wu et al., 1998, Proc. Natl. Acad. Sci. U.S.A. 95:6037-6042.

Anti-uPAR antibodies useful for the uPAR-binding molecule-drug conjugate of the present invention may also be described or specified in terms of their binding affinity to uPAR. Preferred binding affinities include those with a dissociation constant or Kd (K_{off}/Kₒₙ) less than 5×10⁻² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷M, 5×10⁻⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M. In certain embodiments, preferred binding affinities include those with a dissociation constant or Kd more than 10⁻² M, 5×10⁻³ M, 10⁻³ M, 5×10⁻⁴ M, 10⁻⁴ M, 5×10⁻⁵M, 10⁻⁵ M, 5×10⁻⁶ M, 10⁻⁶ M, 5×10⁻⁷ M, 10⁻⁷ M, 5×10⁻⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M. In another embodiment, the K_{off} rate is less than 1×10⁻³ s⁻¹, or less than 3×10⁻³ s⁻¹. In other embodiments, the K_{off} rate is less than 10⁻³ s⁻¹, less than 5×10⁻³ s⁻¹, less than 10⁻⁴ s⁻¹, less than 5×10⁻⁴ s⁻¹, less than 10⁻⁵ s⁻¹, less than 5×10⁻⁵ s⁻¹, less than 10⁻⁶ s⁻¹, less than 5×10⁻⁶ s⁻¹, less than 10⁻⁷ s⁻¹, less than 5×10⁻⁷ s⁻¹, less than 10⁻⁸ s⁻¹, less than 5×10⁻⁸ s⁻¹, less than 10⁻⁹ s⁻¹, less than 5×10⁻⁹ s⁻¹, or less than 10⁻¹⁰ s⁻¹.

In another embodiment, the kₒₙ rate is at least 10⁵ M⁻¹s⁻¹, at least 5×10⁵ M⁻¹s⁻¹, at least 10⁶ M⁻¹s⁻¹, at least 5×10⁶ M⁻¹s⁻¹, at least 10⁷ M⁻¹s⁻¹, at least 5×10⁷ M⁻¹s⁻¹, or at least 10⁸ M⁻¹s⁻¹, or at least 10⁹ M⁻¹s⁻¹.

The anti-uPAR antibodies useful for the uPAR-binding molecule-drug conjugate of the present invention include derivatives that, in addition to conjugation to a drug, are modified, i.e., by the covalent attachment of any type of molecule to the antibody such that covalent attachment does not prevent the antibody from binding to uPAR. For example, but not by way of limitation, the antibody derivatives include antibodies that have been modified, e.g., by glycosylation, acetylation, pegylation, phosphylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, synthesis in the presence of tunicamycin, etc. Additionally, the derivative may contain one or more non-classical amino acids.

The anti-uPAR antibodies useful in the methods and compositions of the present invention may be generated by any suitable method known in the art. Polyclonal antibodies to uPAR can be produced by various procedures well known in the art. For example, uPAR can be administered to various host animals including, but not limited to, rabbits, mice, rats, etc. to induce the production of sera containing polyclonal antibodies specific for the protein. Various adjuvants may be used to increase the immunological response, depending on the host species, and include but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and corynebacterium parvum. Such adjuvants are also well known in the art.

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow, et al., Antibodies: A Laboratory Manual, (Cold Spring Harbor Laboratory Press, 2nd ed., 1988); Hammerling, et al., in: Monoclonal Antibodies and T-Cell Hybridomas 563-681 (Elsevier, N.Y., 1981) (said references incorporated by reference in their entireties). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

Methods for producing and screening for specific antibodies using hybridoma technology are routine and well known in the art. In a non-limiting example, mice can be immunized with uPAR or a fragment or derivative thereof or with a cell expressing said uPAR or uPAR fragment or derivative. Once an immune response is detected, e.g., antibodies specific for uPAR are detected in the mouse serum, the mouse spleen is harvested and splenocytes isolated. The splenocytes are then fused by well known techniques to any suitable myeloma cells, for example cells from cell line SP20 available from the ATCC. Hybridomas are selected and cloned by limited dilution. The hybridoma clones are then assayed by methods known in the art for cells that secrete antibodies capable of binding uPAR. Ascites fluid, which generally contains high levels of antibodies, can be generated by injecting mice with positive hybridoma clones. In a preferred embodiment, the hybridoma is Mof-3, which produces the monoclonal antibody 3936. Hybridoma cell line producing antibodies useful for the present invention have been deposited with the American Type Culture Collection (10801 University Boulevard, Manassas, Va. 20110) on Nov. 16, 1991 under the provisions of the Budapest Treaty on the International Recognition of the Deposit of Microorganisms for the Purposes of Patent Procedures, and assigned accession number ATCC HB-10917 and are incorporated herein by reference. Antibodies that compete with Mab 3936 for the same epitopes are further identified.

Antibody fragments which recognize specific epitopes may be generated by known techniques. For example, Fab and F(ab′)₂ fragments may be produced by proteolytic cleavage of immunoglobulin molecules, using enzymes such as papain (to produce Fab fragments) or pepsin (to produce F(ab′)₂ fragments). F(ab′)₂ fragments contain the variable region, the light chain constant region and the CH 1 domain of the heavy chain.

For example, the anti-uPAR antibodies useful for making the uPAR-binding molecule-drug conjugates of the present invention can also be generated using various phage display methods known in the art. In phage display methods, functional antibody domains are displayed on the surface of phage particles which carry the nucleic acid sequences encoding them. In a particular embodiment, such phage can be utilized to display antigen binding domains expressed from a repertoire or combinatorial antibody library (e.g., human or murine). In phage display methods, functional antibody domains are displayed on the surface of phage particles which carry the nucleic acid sequences encoding them. In particular, DNA sequences encoding V_{H} and V_{L} domains are amplified from animal cDNA libraries (e.g., human or murine cDNA libraries of lymphoid tissues). The DNA encoding the V_{H} and V_{L} domains are recombined together with an scFv linker by PCR and cloned into a phagemid vector (e.g., p CANTAB 6 or pComb 3 HSS). The vector is electroporated in E. coli and the E. coli is infected with helper phage. Phage used in these methods are typically filamentous phage including fd and MI3 binding domains expressed from phage with Fab, Fv or disulfide stabilized Fv antibody domains recombinantly fused to either the phage gene III or gene VIII protein. Phage expressing an antigen binding domain that binds to uPAR can be selected or identified with antigen, e.g., using labeled antigen or antigen bound or captured to a solid surface or bead. Examples of phage display methods that can be used to make the anti-uPAR antibodies of the present invention include those disclosed in Brinkman et al., 1995, J. Immunol. Methods 182:41-50; Ames et al., 1995, J. Immunol. Methods 184:177-186; Kettleborough et al., 1994, Eur. J. Immunol. 24:952-958; Persic et al., 1997, Gene 187:9-18; Burton et al., 1994, Advances in Immunology, 191-280; PCT Application No. PCT/GB91/O1 134; PCT Publications WO 90/02809; WO 91/10737; WO 92/01047; WO 92/18619; WO 93/1 1236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108; each of which is incorporated herein by reference in its entirety.

As described in the above references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria, e.g., as described in detail below. For example, techniques to recombinantly produce Fab, Fab′ and F(ab′)₂ fragments can also be employed using methods known in the art such as those disclosed in PCT publication WO 92/22324; Mullinax et al., BioTechniques 1992, 12(6):864-869; and Sawai et al., 1995, AJRI 34:26-34; and Better et al., 1988, Science 240:1041-1043 (said references incorporated by reference in their entireties).

Examples of techniques which can be used to produce single-chain Fvs and antibodies include those described in U.S. Pat. Nos. 4,946,778 and 5,258,498; Huston et al., 1991, Methods in Enzymology 203:46-88; Shu et al., 1993, PNASi 90:7995-7999; and Skerra et al., 1988, Science 240:1038-1040. For some uses, including in vivo use of antibodies in humans and in vitro proliferation or cytotoxicity assays, it is preferable to use chimeric, humanized, or human antibodies. A chimeric antibody is a molecule in which different portions of the antibody are derived from different animal species, such as antibodies having a variable region derived from a murine monoclonal antibody and a human immunoglobulin constant region. Methods for producing chimeric antibodies are known in the art. See, e.g., Morrison, Science, 1985, 229:1202; Oi et al., 1986, BioTechniques 4:214; Gillies et al., 1989, J. Immunol. Methods 125:191-202; U.S. Pat. Nos. 5,807,715; 4,816,567; and 4,816,397, which are incorporated herein by reference in their entirety. Humanized antibodies are antibody molecules from non-human species antibody that binds the desired antigen having one or more CDRs from the non-human species and framework and constant regions from a human immunoglobulin molecule. Often, framework residues in the human framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; Riechmann et al., 1988, Nature 332:323, which are incorporated herein by reference in their entireties.) Antibodies can be humanized using a variety of techniques known in the art including, for example, CDR-grafting (EP 239,400; PCT publication WO 9 1/09967; U.S. Pat. Nos. 5,225,539; 5,530,101; and 5,585,089), veneering or resurfacing (EP 592,106; EP 519,596; Padlan, Molecular Immunology, 1991, 28(4/5):489-498; Studnicka et al., 1994, Protein Engineering 7(6):805-814; Roguska, et al., 1994, PNAS 91:969-973), and chain shuffling (U.S. Pat. No. 5,565,332).

Completely human antibodies are particularly desirable for therapeutic treatment of human subjects. Human antibodies can be made by a variety of methods known in the art including phage display methods described above using antibody libraries derived from human immunoglobulin sequences. See also, U.S. Pat. Nos. 4,444,887 and 4,716,111; and PCT publications WO 98/46645, WO 98/50433, WO 98/24893, WO 98/16654, WO 96/34096, WO 96/33735, and WO 91/10741; each of which is incorporated herein by reference in its entirety.

Human antibodies can also be produced using transgenic mice which express human immunoglobulin genes. For example, the human heavy and light chain immunoglobulin gene complexes may be introduced randomly or by homologous recombination into mouse embryonic stem cells. The mouse heavy and light chain immunoglobulin genes may be rendered non-functional separately or simultaneously with the introduction of human immunoglobulin loci by homologous recombination. In particular, homozygous deletion of the JH region prevents endogenous antibody production. The modified embryonic stem cells are expanded and microinjected into blastocysts to produce chimeric mice. The chimeric mice are then bred to produce homozygous offspring which express human antibodies. The transgenic mice are immunized in the normal fashion with a selected antigen, e.g., all or a portion of uPAR. Monoclonal antibodies directed against the antigen can be obtained from the immunized, transgenic mice using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar, 1995, Int. Rev. Immunol. 13:65-93. For a detailed discussion of this technology for producing human antibodies and human monoclonal antibodies and protocols for producing such antibodies, see, e.g., PCT publications WO 98/24893; WO 92/01047; WO 96/34096; WO 96/33735; European Patent No. 0 598 877; U.S. Pat. Nos. 5,413,923; 5,625,126; 5,633,425; 5,569,825; 5,661,016; 5,545,806; 5,814,318; 5,885,793; 5,916,771; and 5,939,598, which are incorporated by reference herein in their entirety. In addition, companies such as Abgenix, Inc. (Freemont, Calif.), Genpharm (San Jose, Calif.) and Medarex (Princeton, N.J.) can be engaged to provide human antibodies directed against a selected antigen using technology similar to that described above.

Completely human antibodies which recognize a selected epitope can be generated using a technique referred to as “guided selection.” In this approach a selected non-human monoclonal antibody, e.g., a mouse antibody, is used to guide the selection of a completely human antibody recognizing the same epitope (Jespers et al., 1994, Bio/technology 12:899-903).

In a specific embodiment, the anti-uPAR antibody is a bispecific antibody. In another specific embodiment, the anti-uPAR antibody is not a bispecific antibody. In certain embodiments, the antibody is conjugated to a radioisotope. In more specific embodiments, the radioisotope is ⁹⁰Y (yttrium), ¹¹¹In (indium), ²¹¹At (astatide), ¹³¹I (iodine), ²¹²Bi (bismuth), ²¹³Bi, ²²⁵Ac (actinium), ¹⁸⁶Re (rhenium), ¹⁸⁸Re, ¹⁰⁹Pd (palladium), ⁶⁷Cu (copper), ⁷⁷Br (bromine), ¹⁰⁵Rh (rhodium), ¹⁹⁸Au (gold), ¹⁹⁹Au or ²¹²Pb (lead).

The anti-uPAR antibodies useful in the uPAR-binding molecule-drug conjugate of the present invention may further be recombinantly fused to a heterologous protein at the N- or C-terminus.

5.1.3 Immunobinding Assays

Methods of demonstrating the ability of an antibody to bind to uPAR, and thus its usefulness in the disclosed methods and compositions, are described herein.

A putative anti-uPAR antibody may be assayed for immunospecific binding to uPAR by any method known in the art. The immunoassays which can be used include but are not limited to competitive and non-competitive assay systems using techniques such as Western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, to name but a few. Such assays are routine and well known in the art (see, e.g., Ausubel et. al., eds., 1994, Current Protocols in Molecular Biology, Vol. 1, John Wiley & Sons, Inc., New York, which is incorporated by reference herein in its entirety). Exemplary immunoassays are described briefly below (but are not intended by way of limitation).

Immunoprecipitation protocols generally comprise lysing a population of cells in a lysis buffer such as RIPA buffer (1% NP-40 or Triton X-100, 1% sodium deoxycholate, 0.1% SDS, 0.15 M NaCl, 0.01 M sodium phosphate at pH 7.2, 1% Trasylol) supplemented with protein phosphatase and/or protease inhibitors (e.g., EDTA, PMSF, aprotinin, sodium vanadate), adding the antibody to the cell lysate, incubating for a period of time (e.g., 1-4 hours) at 40° C., adding protein A and/or protein G sepharose beads to the cell lysate, incubating for about an hour or more at 40° C., washing the beads in lysis buffer and resuspending the beads in SDS/sample buffer. The ability of the antibody to immunoprecipitate uPAR can be assessed by, e.g., Western blot analysis. One of skill in the art would be knowledgeable as to the parameters that can be modified to increase the binding of the antibody to uPAR and decrease the background (e.g., pre-clearing the cell lysate with sepharose beads). For further discussion regarding immunoprecipitation protocols see, e.g., Ausubel et al. eds., 1994, Current Protocols in Molecular Biology, Vol. 1, John Wiley & Sons, Inc., New York at 10.16.1.

Western blot analysis generally comprises preparing protein samples, electrophoresis of the protein samples in a polyacrylamide gel (e.g., 8%-20% SDS-PAGE depending on the molecular weight of the antigen), transferring the protein sample from the polyacrylamide gel to a membrane such as nitrocellulose, PVDF or nylon, incubating the membrane in blocking solution (e.g., PBS with 3% BSA or non-fat milk), washing the membrane in washing buffer (e.g., PBS-Tween 20), blocking the membrane with primary antibody (i.e., the putative anti-uPAR antibody) diluted in blocking buffer, washing the membrane in washing buffer, incubating the membrane with a secondary antibody (which recognizes the primary antibody, e.g., an anti-human antibody) conjugated to an enzyme substrate (e.g., horseradish peroxidase or alkaline phosphatase) or radioactive molecule (e.g., ³²P or ¹²⁵I) diluted in blocking buffer, washing the membrane in wash buffer, and detecting the presence of the secondary antibody. One of skill in the art would be knowledgeable as to the parameters that can be modified to increase the signal detected and to reduce the background noise. For further discussion regarding Western blot protocols see, e.g., Ausubel et al. eds., 1994, Current Protocols in Molecular Biology, Vol. 1, John Wiley & Sons, Inc., New York at 10.8.1.

ELISAs comprise preparing antigen (i.e., uPAR), coating the well of a 96 well microtiter plate with the uPAR, adding the antibody conjugated to a detectable compound such as an enzyme (e.g., horseradish peroxidase or alkaline phosphatase) to the well and incubating for a period of time, and detecting the presence of the antibody. In ELISAs the antibody does not have to be conjugated to a detectable compound; instead, a second antibody (which recognizes the antibody of interest) conjugated to a detectable compound may be added to the well. Further, instead of coating the well with the antigen, the antibody may be coated to the well. In this case, a second antibody conjugated to a detectable compound may be added following the addition of uPAR protein to the coated well. One of skill in the art would be knowledgeable as to the parameters that can be modified to increase the signal detected as well as other variations of ELISAs known in the art. For further discussion regarding ELISAs see, e.g., Ausubel et al., eds., 1994, Current Protocols in Molecular Biology, Vol. 1, John Wiley & Sons, Inc., New York at 11.2.1.

The binding affinity of an antibody to uPAR and the off-rate of an antibody-uPAR interaction can be determined by competitive binding assays. One example of a competitive binding assay is a radioimmunoassay comprising the incubation of labeled uPAR (e.g., ³H or ¹²⁵I) with the antibody of interest in the presence of increasing amounts of unlabeled uPAR, and the detection of the antibody bound to the labeled uPAR. The affinity of the antibody for uPAR and the binding off-rates can then be determined from the data by Scatchard plot analysis. Competition of a first antibody with a second antibody can also be determined using radioimmunoassays. In this case, uPAR is incubated with the antibody of interest conjugated to a labeled compound (e.g., ³H or ¹²⁵I) in the presence of increasing amounts of an unlabeled second antibody. In a specific embodiment, the first antibody is Mab 3936. In a specific embodiment, the second antibody is Mab 3936.

5.1.3.1 Methods of Producing Anti-uPAR Antibodies

The anti-uPAR antibodies useful for the uPAR-binding molecule-drug conjugate of the invention can be produced by any method known in the art for the synthesis of proteins, in particular, by chemical synthesis or preferably, by recombinant expression techniques.

Recombinant expression of an anti-uPAR antibody, including a fragment, derivative or analog thereof, e.g., a heavy or light chain of an anti-uPAR antibody, requires construction of an expression vector containing a nucleic acid that encodes the anti-uPAR antibody. Once a nucleic acid encoding an anti-uPAR antibody has been obtained, the vector for the production of the anti-uPAR antibody may be produced by recombinant DNA technology using techniques well known in the art. Thus, methods for preparing an anti-uPAR antibody by expressing a nucleic acid containing a nucleotide sequence encoding said anti-uPAR antibody are described herein. Methods which are well known to those skilled in the art can be used to construct expression vectors containing coding sequences and appropriate transcriptional and translational control signals. These methods include, for example, in vitro recombinant DNA techniques, synthetic techniques, and in vivo genetic recombination. The invention, thus, provides replicable vectors comprising a nucleotide sequence encoding an anti-uPAR antibody operably linked to a promoter. The anti-uPAR antibody nucleotide sequence may encode a heavy or light chain thereof, or a heavy or light chain variable domain, operably linked to a promoter. Such vectors may include the nucleotide sequence encoding the constant region of the anti-uPAR antibody molecule (see, e.g., PCT Publication WO 86/05807; PCT Publication WO 89/01036; and U.S. Pat. No. 5,122,464) and the variable domain of the anti-uPAR antibody may be cloned into such a vector for expression of the entire heavy or light chain.

The expression vector is transferred to a host cell by conventional techniques and the transfected cells are then cultured by conventional techniques to produce a protein of the invention. Thus, the invention encompasses host cells containing a nucleic acid encoding a protein of the invention, operably linked to a heterologous promoter. In preferred embodiments for the expression of double-chained antibodies, vectors encoding both the heavy and light chains may be co-expressed in the host cell for expression of the entire immunoglobulin molecule, as detailed below.

A variety of host-expression vector systems may be utilized to express the protein molecules of the invention. Such host-expression systems represent vehicles by which the coding sequences of interest may be produced and subsequently purified, but also represent cells which may, when transformed or transfected with the appropriate nucleotide coding sequences, express a protein of the invention in situ. These include but are not limited to microorganisms such as bacteria (e.g., E. coli, B. subtilis) transformed with recombinant bacteriophage DNA, plasmid DNA or cosmid DNA expression vectors containing antibody coding sequences; yeast (e.g., Saccharomyces, Pichia) transformed with recombinant yeast expression vectors containing antibody coding sequences; insect cell systems infected with recombinant virus expression vectors (e.g., baculovirus) containing antibody coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS, CHO, BHK, 293, 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). Preferably, bacterial cells such as Escherichia coli, and more preferably, eukaryotic cells, especially for the expression of whole recombinant antibody molecules, are used for the expression of a recombinant protein of the invention. For example, mammalian cells such as Chinese hamster ovary cells (CHO), in conjunction with a vector such as the major intermediate early gene promoter element from human cytomegalovirus is an effective expression system for proteins of the invention (Foecking et al., 1986, Gene 45:101; Cockett et al., 1990, Bio/Technology 8:2).

In bacterial systems, a number of expression vectors may be advantageously selected depending upon the use intended for the folding and post-translation modification requirements protein being expressed. Where possible, when a large quantity of an anti-uPAR antibody is to be produced, for the generation of the anti-uPAR ADCs of the invention or pharmaceutical compositions comprising such ADCs, vectors which direct the expression of high levels of fusion protein products that are readily purified may be desirable. Such vectors include, but are not limited, to the E. coli expression vector pUR278 (Ruther et al., 1983, EMBO 1. 2:1791), in which the anti-uPAR antibody coding sequence may be ligated individually into the vector in frame with the lac Z coding region so that a fusion protein is produced; pIN vectors (Inouye & Inouye, 1985, Nucleic Acids Res. 13:3101-3109; Van Heeke & Schuster, 1989, J. Biol. Chem. 24:5503-5509); and the like. pGEX vectors may also be used to express fusion proteins with glutathione S-transferase (GST). In general, such fusion proteins are soluble and can easily be purified from lysed cells by adsorption and binding to matrix glutathioneagarose beads followed by elution in the presence of free glutathione. The pGEX vectors are designed to include thrombin or factor Xa protease cleavage sites so that the cloned anti-uPAR antibody can be released from the GST moiety.

In an insect system, Autographa californica nuclear polyhedrosis virus (AcNPV) is used as a vector to express foreign genes. The virus grows in Spodoptera frugiperda cells. The anti-uPAR antibody coding sequence may be cloned individually into non-essential regions (for example the polyhedrin gene) of the virus and placed under control of an AcNPV promoter (for example the polyhedrin promoter).

In mammalian host cells, a number of viral-based expression systems may be utilized. In cases where an adenovirus is used as an expression vector, the coding sequence of the anti-uPAR antibody may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the anti-uPAR antibody in infected hosts. (See, e.g., Logan & Shenk, 1984, Proc. Natl. Acad. Sci. USA 8 1:355-359). Specific initiation signals may also be required for efficient translation of inserted coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see Bittner et al., 1987, Methods in Enzymol. 153:51-544).

In addition, a host cell strain may be chosen which modulates the expression of the inserted sequences, or modifies and processes the gene product in the specific fashion desired. Such modifications (e.g., glycosylation) and processing (e.g., cleavage) of anti-uPAR antibodies may be important for the binding and/or activities of the antibodies. Different host cells have characteristic and specific mechanisms for the post-translational processing and modification of proteins and gene products. Appropriate cell lines or host systems can be chosen to ensure the correct modification and processing of the anti-uPAR antibody expressed. To this end, eukaryotic host cells which possess the cellular machinery for proper processing of the primary transcript, glycosylation, and phosphorylation of the gene product may be used. Such mammalian host cells include but are not limited to CHO, VERO, BHK, Hela, COS, MDCK, 293, 3T3, and W138.

For long-term, high-yield production of recombinant anti-uPAR antibodies, stable expression is preferred. For example, cell lines which stably express an anti-uPAR antibody may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express an anti-uPAR antibody for use in the methods of the present invention.

A number of selection systems may be used, including but not limited to the herpes simplex virus thymidine kinase (Wigler et al., 1977, Cell 11:223), hypoxanthine guanine phosphoribosyltransferase (Szybalska & Szybalski, 1992, Proc. Natl. Acad. Sci. USA 48:202), and adenine phosphoribosyltransferase (Lowy et al., 1980, Cell 22:8-17) genes can be employed in tk−, hgprt− or aprt− cells, respectively. Also, antimetabolite resistance can be used as the basis of selection for the following genes: dhfr, which confers resistance to methotrexate (Wigler et al., 1980, Proc. Natl. Acad. Sci. USA 77:357; O'Hare et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Clinical Pharmacy 12:488-505; Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62: 191-217; May, 1993, TIB TECH 11(5):155-215); and hygro, which confers resistance to hygromycin (Santerre et al., 1984, Gene 30:147). Methods commonly known in the art of recombinant DNA technology may be routinely applied to select the desired recombinant clone, and such methods are described, for example, in Ausubel et al. (eds.), Current Protocols in Molecular Biology, John Wiley & Sons, NY (1993); Kriegler, Gene Transfer and Expression, A Laboratory Manual, Stockton Press, NY (1990); and in Chapters 12 and 13, Dracopoli et al. eds., Current Protocols in Human Genetics, John Wiley & Sons, NY (1994); Colberre-Garapin et al., 1981, J. Mol. Biol. 150: 1, which are incorporated by reference herein in their entireties.

The expression levels of an anti-uPAR antibody can be increased by vector amplification (for a review, see Bebbington and Hentschel, “The Use of Vectors Based on Gene Amplification for the Expression of Cloned Genes in Mammalian Cells in DNY Cloning”, Vol. 3. (Academic Press, New York, 1987)). When a marker in the vector system expressing the anti-uPAR antibody is amplifiable, increase in the level of inhibitor present in culture of host cell will increase the number of copies of the marker gene. Since the amplified region is associated with the anti-uPAR antibody gene, production of the anti-uPAR antibody will also increase (Crouse et al., 1983, Mol. Cell. Biol. 3:257).

In certain specific embodiments, the host cell may be co-transfected with two expression vectors encoding an anti-uPAR antibody, the first vector encoding a heavy chain derived protein and the second vector encoding a light chain derived protein. The two vectors may contain identical selectable markers which enable equal expression of heavy and light chain proteins. Alternatively, a single vector may be used which encodes, and is capable of expressing, both heavy and light chain proteins. In such situations, the light chain should be placed before the heavy chain to avoid an excess of toxic free heavy chain (Proudfoot, 1986, Nature 322:52 (1986); Kohler, 1980, Proc. Natl. Acad. Sci. USA 77:2197). The coding sequences for the heavy and light chains may comprise cDNA or genomic DNA.

Once an anti-uPAR antibody has been produced by an animal, chemically synthesized, or recombinantly expressed, it may be purified by any method known in the art for purification of proteins, for example, by chromatography (e.g., ion exchange; affinity, particularly by affinity for the specific antigen (i.e., uPAR); Protein A; or affinity for a heterologous fusion partner wherein the protein is a fusion protein; and sizing column chromatography), centrifugation, differential solubility, or by any other standard technique for the purification of proteins.

5.1.4 Formation of uPAR-Binding Molecule Drug Conjugates

The generation of uPAR-binding molecule-drug conjugate can be accomplished by any technique known to the skilled artisan. Briefly, the uPAR-binding molecule-drug conjugate comprises a uPAR-binding molecule, a drug, and a linker that joins the drug and the uPAR-binding molecule. The uPAR-binding molecule can be antibodies, peptides, in particular, peptide mimetics, peptide derivatives, peptide analogs, or non-peptide mimetics. A number of different reactions are available for covalent attachment of drugs to the uPAR-binding molecule. For antibodies, peptides, peptide derivatives, peptide analogs, this is often accomplished by reaction of the amino acid residues of the antibody molecule or the peptide, peptide derivative, or peptide analog, including the amine groups of lysine, the free carboxylic acid groups of glutamic and aspartic acid, the sulfhydryl groups of cysteine and the various moieties of the aromatic amino acids. For antibody, one of the most commonly used non-specific methods of covalent attachment is the carbodiimide reaction to link a carboxy (or amino) group of a compound to amino (or carboxy) groups of the antibody. Additionally, bifunctional agents such as dialdehydes or imidoesters have been used to link the amino group of a compound to amino groups of the antibody molecule. Also available for attachment of drugs to antibodies is the Schiff base reaction. This method involves the periodate oxidation of a drug that contains glycol or hydroxy groups, thus forming an aldehyde which is then reacted with the antibody molecule. Attachment occurs via formation of a Schiff base with amino groups of the antibody. Isothiocyanates can also be used as coupling agents for covalently attaching drugs to antibodies. In a specific embodiment, the uPAR-binding molecule-drug conjugate is a fusion protein. In a preferred embodiment, the molecule of the invention is a fusion protein comprising a uPAR-binding molecule and a human perforin or a fragment thereof. Other techniques known to the skilled artisan and within the scope of the present invention. Non-limiting examples of such techniques are described in, e.g., U.S. Pat. Nos. 5,665,358, 5,643,573, and 5,556,623, which are incorporated by reference in their entireties herein.

In certain embodiments, an intermediate, which is the precursor of the linker, is reacted with the drug under appropriate conditions. In certain embodiments, reactive groups are used on the drug and/or the intermediate. The product of the reaction between the drug and the intermediate, or the derivatized drug, is subsequently reacted with the uPAR-binding molecule under appropriate conditions. Care should be taken to maintain the stability of the uPAR-binding molecule under the conditions chosen for the reaction between the derivatized drug and the uPAR-binding molecule.

5.1.5 Linkers

uPAR-binding molecule are recombinantly fused or chemically conjugated (including both covalent and non-covalent conjugations) to a drug. In specific embodiments, the uPAR-binding molecule of a uPAR-binding molecule-drug conjugate of the invention is conjugated to the drug directly without a linker. In specific embodiments, the uPAR-binding molecule-drug conjugate of the invention is conjugated to the drug via a covalent bond. In other embodiments, the uPAR-binding molecule-drug conjugate of the invention is conjugated to the chemotherapeutic via a non-covalent bond. In other specific embodiments, the uPAR-binding molecule of a uPAR-binding molecule-drug conjugate of the invention is conjugated to the drug via a linker. In certain embodiments, the linker is a biodegradable linker; in other embodiments, the linker is a non-biodegradable linker. In a preferred embodiment, the linker is a peptide linker. In specific embodiments, the linker is a hydrazone linker, or a disulfide linker.

A majority of the conjugates of the present invention, which comprise a uPAR-binding molecule and a drug, further comprise a linker. Any linker that is known in the art may be used in the uPAR-binding molecule-drug conjugates of the present invention, e.g., bifunctional agents (such as dialdehydes or imidoesters) or branched hydrazone linkers (see, e.g., U.S. Pat. No. 5,824,805, which is incorporated by reference herein in its entirety).

Techniques for conjugating prophylacetic or therapeutic moieties to antibodies are well known. Moieties can be conjugated to antibodies by any method known in the art, including, but not limited to aldehyde/Schiff linkage, sulphydryl linkage, acid-labile linkage, cis-aconityl linkage, hydrazone linkage, enzymatically degradable linkage (see generally Garnett, 2002, Adv. Drug Deliv. Rev. 53:171-216). Additional techniques for conjugating prophylacetic or therapeutic moieties to antibodies are well known, see, e.g., Arnon et al., “Monoclonal Antibodies For Immunotargeting Of Drugs In Cancer Therapy,” in Monoclonal Antibodies And Cancer Therapy, Reisfeld et al. (eds.), pp. 243-56 (Alan R. Liss, Inc. 1985); Hellstrom et al., “Antibodies For Drug Delivery,” in Controlled Drug Delivery (2nd ed.), Robinson et al. (eds.), pp. 623-53 (Marcel Dekker, Inc. 1987); Thorpe, “Antibody Carriers Of Cytotoxic Agents In Cancer Therapy: A Review,” in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy,” in Monoclonal Antibodies For Cancer Detection And Therapy, Baldwin et al. (eds.), pp. 303-16 (Academic Press 1985), and Thorpe et al., 1982, Immunol. Rev. 62:119-58. Methods for fusing or conjugating antibodies to polypeptide moieties are known in the art. See, e.g., U.S. Pat. Nos. 5,336,603, 5,622,929, 5,359,046, 5,349,053, 5,447,851, and 5,112,946; EP 307,434; EP 367,166; International Publication Nos. WO 96/04388 and WO 91/06570; Ashkenazi et al., 1991, PNAS 88: 10535-10539; Zheng et al., 1995, J. Immunol. 154:5590-5600; and Vil et al., 1992, PNAS 89:11337-11341. The fusion of an antibody to a moiety does not necessarily need to be direct, but may occur through linker sequences. Such linker molecules are commonly known in the art and described in Denardo et al., 1998, Clin Cancer Res. 4:2483-90; Peterson et al., 1999, Bioconjug. Chem. 10:553; Zimmerman et al., 1999, Nucl. Med. Biol. 26:943-50; Garnett, 2002, Adv. Drug Deliv. Rev. 53:171-216.

In certain, non-limiting, embodiments of the invention, the linker region between the drug moiety and the uPAR-binding moiety of the uPAR-binding molecule-drug conjugate is cleavable or hydrolyzable under certain conditions, wherein cleavage or hydrolysis of the linker releases the drug moiety from the uPAR-binding moiety. Preferably, the linker is sensitive to cleavage or hydrolysis under intracellular conditions.

In a preferred embodiment, the linker region between the drug moiety and the uPAR-binding moiety of the uPAR-binding molecule-drug conjugate is hydrolyzable if the pH changes by a certain value or exceeds a certain value. In a particularly preferred embodiment of the invention, the linker is hydrolyzable in the milieu of the lysosome, e.g., under acidic conditions (i.e., a pH of around 5-5.5 or less). In other embodiments, the linker is a peptidyl linker that is cleaved by a peptidase or protease enzyme, including but not limited to a lysosomal protease enzyme, a membrane-associated protease, an intracellular protease, or an endosomal protease. Preferably, the linker is at least two amino acids long, more preferably at least three amino acids long. Peptidyl linkers that are cleavable by enzymes that are present in cancers involving or mediated by uPAR-expressing cells are preferred. For example, a peptidyl linker that is cleavable by cathepsin-B (e.g., a Gly-Phe-Leu-Gly linker), a thiol-dependent protease that is highly expressed in cancerous tissue, can be used. Other such linkers are described, e.g., in U.S. Pat. No. 6,214,345, which is incorporated by reference in its entirety herein.

In other, non-mutually exclusive embodiments of the invention, the linker by which the anti-uPAR antibody and the drug of a uPAR-binding molecule-drug conjugate of the invention are conjugated to promotes cellular internalization. In certain embodiments, the linker-drug moiety of the uPAR-binding molecule-drug conjugate promotes cellular internalization. In certain embodiments, the linker is chosen such that the structure of the entire uPAR-binding molecule drug conjugate promotes cellular internalization. In certain embodiments, the linker is a cell surface receptors, growth hormone receptors, synthetic receptor, ubiquitin, or fragments thereof. In specific embodiments, the linker utilizes synthetic receptor targeting strategies. (See Peterson, 2005, Org. Biomol. Chem. 3:3607-3612). In specific embodiments, the uPAR-binding molecule-drug conjugates of the present invention is modified by ubiquitylation. (See Shih et al., 2000, EMBO J. 19(2):187-198).

Moreover, the uPAR-binding molecule can be conjugated to therapeutic moieties such as a radioactive materials or macrocyclic chelators useful for conjugating radiometal ions (see above for examples of radioactive materials). In certain embodiments, the macrocyclic chelator is 1,4,7,10-tetraazacyclododecane-N,N′,N″,N″-tetraacetic acid (DOTA) which can be attached to the uPAR-binding molecule, such as an anti-uPAR antibody via a linker molecule. Such linker molecules are commonly known in the art and described in Denardo et al., 1998, Clin Cancer Res. 4:2483-90; Peterson et al., 1999, Bioconjug. Chem. 10:553; and Zimmerman et al., 1999, Nucl. Med Biol. 26:943-50 each incorporated by reference in their entireties.

In a specific embodiment of the invention, derivatives of valine-citrulline are used as linker (val-cit linker). The synthesis of doxorubicin with the val-cit linker have been previously described (U.S. Pat. No. 6,214,345 to Dubowchik and Firestone, which is incorporated by reference herein in its entirety).

In another specific embodiment, the linker is a phe-lys linker.

In another specific embodiment, the linker is a thioether linker (see, e.g., U.S. Pat. No. 5,622,929 to Willner et al., which is incorporated by reference herein in its entirety).

In yet another specific embodiment, the linker is a hydrazone linker (see, e.g., U.S. Pat. Nos. 5,122,368 to Greenfield et al., 5,824,805 to King et al., 5,137,877 to Kaneko et al., which are incorporated by reference herein in their entireties). In a preferred embodiment, the linker is (6-Maleimidocaproyl) hydrazone (see, e.g., Willner et al., 1993, Bioconjugate Chem. 4:521)

In yet other specific embodiments, the linker is a disulfide linker. A variety of disulfide linkers are known in the art, including but not limited to those that can be formed using SATA (N-succinimidyl-5-acetylthioacetate), SPDP (N-succinimidyl-3-(2-pyridyldithio)propionate), SPDB (N-succinimidyl-3-(2-pyridyldithio)butyrate) and SMPT (N-succinimidyl-oxycarbonyl-alpha-methyl-alpha-(2-pyridyl-dithio)toluene). SPDB and SMPT (see, e.g., Thorpe et al., 1987, Cancer Res., 47:5924-5931; Wawrzynczak et al., 1987, In Immunoconjugates: Antibody Conjugates in Radioimagery and Therapy of Cancer, ed. C. W. Vogel, Oxford U. Press, pp. 28-55; see also U.S. Pat. No. 4,880,935 to Thorpe et al., which is incorporated by reference herein in its entirety).

In other embodiments, the uPAR-binding molecule is attached to the drug directly without a linker. In a specific embodiment, the uPAR-binding molecule is attached to the drug via a covalent bond. In another specific embodiment, the uPAR-binding molecule is attached to the drug via a non-covalent bond.

In certain embodiments, the uPAR-binding molecule of the uPAR-binding molecule-drug conjugate is conjugated indirectly to a drug through a protein or a peptide. In specific embodiments, the protein is an inhibitor of the uPAR-binding molecule. In specific embodiments, the inhibitor of the uPAR-binding molecule is PAI-1 or PAI-2. In specific embodiments, the peptide is a fragment, derivative or analog of an inhibitor of the uPAR-binding molecule. In specific embodiments, the peptide is a fragment, derivative or analog of PAI-1 or PAI-2.

In certain embodiment, the uPAR-binding molecule-drug conjugate comprises a uPAR-binding molecule conjugated to an inhibitor of the uPAR-binding molecule, said inhibitor of the uPAR-binding molecule is conjugated to a drug.

In certain embodiments, the present invention is directed to a conjugate molecule comprising a uPA inhibitor conjugated to a drug.

In a specific embodiment, the present invention is directed to a conjugate molecule comprising a PAI-1 conjugated to doxorubicin. In another specific embodiment, the conjugate molecule comprising a PAI-2 conjugated to doxorubicin.

In a specific embodiment, the uPAR-binding molecule is conjugated to (6-maleimidocaproyl) hydrazone of doxorubicin.

5.1.6 Therapeutic Agents

The present invention encompasses compositions comprising uPAR-binding-drug conjugate comprising a uPAR-binding molecule conjugated to a prophylacetic or therapeutic agent, where the prophylacetic or therapeutic agent is capable of exerting a cytotoxic or cytostatic effect on a uPAR-expressing cell. In preferred embodiments, the cytotoxic or cytostatic effect is selective for cancer cells. In preferred embodiments, the therapeutic agent is a chemotherapeutic agent. In preferred embodiments, the therapeutic agent is not an antimetabolite.

As used herein, the prophylacetic or therapeutic agent is a cytotoxin, e.g., a cytostatic or cytocidal agent, a therapeutic agent or a radioactive metal ion, e.g., alpha-emitters. A cytotoxin or cytotoxic agent includes any agent that is detrimental to cells. In an embodiment, the prophylacetic or therapeutic agent is not a radioisotope.

The uPAR-binding molecule-drug conjugate of the invention are tailored to produce clinically beneficial cytotoxic or cytostatic effects on uPAR-expressing cells when administered to a patient with a cancer involving or mediated by uPAR-expressing cells, preferably when administered alone but also in combination with other therapeutic agents. Such cytotoxic or cytostatic effects can be achieved by use of a uPAR-binding molecule-drug conjugate that is capable of accumulating inside the uPAR-expressing cell of the anti-uPAR antibody.

In specific embodiments, the therapeutic moieties is an anti-cancer agent, which includes, but not limited to: acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bisphosphonates (e.g., pamidronate (Aredria), sodium clondronate (Bonefos), zoledronic acid (Zometa), alendronate (Fosamax), etidronate, ibandomate, cimadronate, risedromate, and tiludromate); bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alpha-2a; interferon alpha-2b; interferon alpha-n1; interferon alpha-n3; interferon beta-Ia; interferon gamma-Ib; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; anti-CD2 antibodies; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride.

In specific embodiments, the anti-cancer agent includes, but not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lac tam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, dioxamycin; diphenyl spiromustine; docetaxel; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; HMG CoA reductase inhibitors (e.g., atorvastatin, cerivastatin, fluvastatin, lescol, lupitor, lovastatin, rosuvastatin, and simvastatin); hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-iroplact; irsogladine; isobengazole; isohomohalicondrin B; itasetron; jasplakinolide; kahalalide F; lamellarini-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; LFA-3TIP; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin a nalogues; mitonafide; mitotoxin fibroblast growth factor-saporin; mitoxantrone; mofarotene; molgramostim; monoclonal antibody, human chorionic gonadotrophin; monophosphoryl lipid A+mycobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; 5-fluorouracil; leucovorin; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; thalidomide; velaresol; veramine; verdins; verteporfin; vinorelbine; vinxaltine; vorozole; zanoterone; zeniplatin; zilascorb; and zinostatin stimalamer.

Cytotoxic agents useful for the present invention may include saporin, A-chain ricin, A-chain cholera toxin, antibiotic, ricin, ribotoxins, Pseudomonas exotoxin A, Diphtheria toxin, and their truncated derivatives. Other useful cytotoxic agents include perforin. In other embodiments, the cytotoxic agent is not saporin, A-chain ricin, A-chain cholera toxin, antibiotic, ricin, ribotoxins, Pseudomonas exotoxin A, Diphtheria toxin, or their truncated derivatives. In other embodiments, the cytotoxic agent is not an antimetabolite. In a preferred embodiment, the cytotoxic agent is human perforin, fragments, derivatives and analogs thereof. In preferred embodiments, the human perforin fragment useful for the present invention is amino acid residues 1-34 of human perforin.

In preferred embodiments, therapeutic moieties include, but are not limited to, antimetabolites (e.g., methotrexate, 6-mercaptopurine, 6-thioguanine, cytarabine, 5-fluorouracil decarbazine); alkylating agents (e.g., mechlorethamine, thioepa chlorambucil, melphalan, carmustine (BCNU) and lomustine (CCNU), cyclophosphamide, busulfan, dibromomannitol, streptozotocin, mitomycin C, and cisdichlorodiamine platinum(II) (DDP), and cisplatin); anthracyclines (e.g., daunorubicin (formerly daunomycin) and doxorubicin); antibiotics (e.g., dactinomycin (formerly actinomycin), bleomycin, mithramycin, and anthramycin (AMC)); Auristatin molecules (e.g., auristatin PHE, bryostatin 1, and solastatin 10; see Woyke et al., Antimicrob. Agents Chemother. 46:3802-8 (2002), Woyke et al., Antimicrob. Agents Chemother. 45:3580-4 (2001), Mohammad et al., Anticancer Drugs 12:735-40 (2001), Wall et al., Biochem. Biophys. Res. Commun. 266:76-80 (1999), Mohammad et al., Int. J. Oncol. 15:367-72 (1999); DNA-repair enzyme inhibitors (e.g., etoposide or topotecan), kinase inhibitors (e.g., compound ST1571, imatinib mesylate (Kantaijian et al., Clin Cancer Res. 8(7):2167-76 (2002)); cytotoxic agents (e.g., paclitaxel, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucorticoids, procaine, tetracaine, lidocaine, propranolol, and puromycin and analogs or homologs thereof and those compounds disclosed in U.S. Pat. Nos. 6,245,759, 6,399,633, 6,383,790, 6,335,156, 6,271,242, 6,242,196, 6,218,410, 6,218,372, 6,057,300, 6,034,053, 5,985,877, 5,958,769, 5,925,376, 5,922,844, 5,911,995, 5,872,223, 5,863,904, 5,840,745, 5,728,868, 5,648,239, 5,587,459); farnesyl transferase inhibitors (e.g., R115777, BMS-214662, and those disclosed by, for example, U.S. Pat. Nos. 6,458,935, 6,451,812, 6,440,974, 6,436,960, 6,432,959, 6,420,387, 6,414,145, 6,410,541, 6,410,539, 6,403,581, 6,399,615, 6,387,905, 6,372,747, 6,369,034, 6,362,188, 6,342,765, 6,342,487, 6,300,501, 6,268,363, 6,265,422, 6,248,756, 6,239,140, 6,232,338, 6,228,865, 6,228,856, 6,225,322, 6,218,406, 6,211,193, 6,187,786, 6,169,096, 6,159,984, 6,143,766, 6,133,303, 6,127,366, 6,124,465, 6,124,295, 6,103,723, 6,093,737, 6,090,948, 6,080,870, 6,077,853, 6,071,935, 6,066,738, 6,063,930, 6,054,466, 6,051,582, 6,051,574, and 6,040,305); topoisomerase inhibitors (e.g., camptothecin; irinotecan; SN-38; topotecan; 9-aminocamptothecin; GG-211 (GI 147211); DX-8951f; IST-622; rubitecan; pyrazoloacridine; XR-5000; saintopin; UCE6; UCE1022; TAN-1518A; TAN-1518B; KT6006; KT6528; ED-110; NB-506; ED-110; NB-506; and rebeccamycin); bulgarein; DNA minor groove binders such as Hoechst dye 33342 and Hoechst dye 33258; nitidine; fagaronine; epiberberine; coralyne; beta-lapachone; BC-4-1; bisphosphonates (e.g., alendronate, cimadronate, clodronate, tiludronate, etidronate, ibandronate, neridronate, olpandronate, risedronate, piridronate, pamidronate, zoledronate); HMG-CoA reductase inhibitors, (e.g., lovastatin, simvastatin, atorvastatin, pravastatin, fluvastatin, statin, cerivastatin, lescol, lupitor, rosuvastatin and atorvastatin); antisense oligonucleotides (e.g., those disclosed in the U.S. Pat. Nos. 6,277,832, 5,998,596, 5,885,834, 5,734,033, and 5,618,709); adenosine deaminase inhibitors (e.g., Fludarabine phosphate and 2-Chlorodeoxyadenosine); ibritumomab tiuxetan (Zevalin®); tositumomab (Bexxar®)) and pharmaceutically acceptable salts, solvates, clathrates, and prodrugs thereof.

In other embodiments, the therapeutic agent is not an antimetabolite. In preferred embodiments, the therapeutic agent is not a folic acid antagonist, purine antagonist or a pyrimidine antagonist. In specific embodiments, the therapeutic agent is not mercaptopurine, (6-MP, Purinethol), thioguanine (6-TG), Fluorouracil (5-FU), cytarabine (cytosine arabinoside, ara-C), or azacitidine (5-azacytidine)

Therapeutic moieties or drug moieties are not to be construed as limited to classical chemical therapeutic agents. For example, the drug moiety may be a protein, peptide, or polypeptide possessing a desired biological activity. Such proteins may include, for example, a toxin such as abrin, ricin A, pseudomonas exotoxin, cholera toxin, or diphtheria toxin.

In a preferred embodiment, the therapeutic agent is doxorubicin. Doxorubicin is an anthracyclin antibiotic originally isolated from Streptomyces peucetius var caesius (F. Arcamone et al., U.S. Pat. No. 3,590,028 (1971 to Farmitalia); F. Arcamone et al., U.S. Pat. No. 3,803,124 (1974 to Farmitalia)) from which derivatives have been synthesized (F. Arcamone et al., German Patent DE 1 917 874 (1969 to Farmitalia); F. Arcamone et al., U.S. Pat. No. 3,590,028 (1971 to Farmitalia)). Also known as 14-hydroxydaunomycin and adriamycin (the former generic name) doxorubicin has been synthesized from daunomycin and from 7-deoxydaunomycinone (F. Arcamone (1969) Chem. Ind. (Milan) 51:834; E. M. Acton (1974) et al., J. Med. Chem. 17:659; T. H. Smith U.S. Pat. No. 4,012,448 (1977 to Stanford Research Inst.)).

### 5.2 Therapeutic Methods

The present invention provides methods of treating, managing or ameliorating cancers that expresses uPAR (including, but not limited to, cancer of the breast, prostate, ovary, lung, colon, pancreas, and bladder) by administering to a subject in need thereof an effective amount of a uPAR-binding molecule-drug conjugate of the invention. In certain embodiments, the subject has benign, malignant or metastatic cancer or malignant cancer. In other embodiments, the cancer has metastasized to sites distal to the primary cancer. In another embodiment, the uPAR-binding molecule-drug conjugate of the invention can be administered in combination with one or more other therapeutic agents. The subject is preferably a mammal such as non-primate (e.g., cows, pigs, horses, cats, dogs, rats, etc.) and primate (e.g., monkey and a human). In a preferred embodiment, the subject is a human.

The present invention also provides methods to treat, manage, or ameliorate cancer in patients suffering from or expected to suffer from cancer, e.g., have a genetic predisposition for a cancer or are likely of recurrence of cancer. The present invention also provides methods to treat, manage, or ameliorate cancers in patients that are refractory to other treatments or cannot tolerate other treatment because of side effects. The present invention also includes combination of methods of the present invention with surgery, standard and experimental chemotherapies, hormonal therapies, biological therapies/immunotherapies and/or radiation therapies for treatment or prevention of cancer.

5.2.1 Compositions and Methods of Administration

5.2.1.1 Pharmaceutical Compositions

The pharmaceutical compositions (i.e., compositions that are suitable for administration to a subject or patient) can be used in the preparation of unit dosage forms. Such compositions comprise a therapeutically effective amount of a therapeutic agent disclosed herein or a combination of those agents and a pharmaceutically acceptable carrier. Preferably, compositions of the invention comprise a therapeutically effective amount of a uPAR-binding molecule-drug of the invention and a pharmaceutically acceptable carrier. In a further embodiment, the composition of the invention further comprises an additional anti-cancer agent.

In a specific embodiment, the term “pharmaceutically acceptable” means approved by a regulatory agency of the Federal or a state government or listed in the U.S. Pharmacopoeia or other generally recognized pharmacopoeia for use in animals, and more particularly in humans. The term “carrier” refers to a diluent, adjuvant (e.g., Freund's adjuvant (complete and incomplete), excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like.

Generally, the ingredients of compositions of the invention are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compositions of the invention can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

Various delivery systems are known and can be used to administer a uPAR-binding molecule-drug conjugate of the invention or in combination with a therapeutic agent useful for treating cancer, e.g., encapsulation in liposomes, microparticles, microcapsules. Methods of administering a therapeutic agent of the invention include, but are not limited to, parenteral (e.g., intradermal, intramuscular, intraperitoneal, intravenous and subcutaneous), intratumoral, epidural, and mucosal (e.g., intranasal, inhaled, and oral routes) administration. In a specific embodiment, prophylacetic or therapeutic agents of the invention are administered intramuscularly, intravenously, or subcutaneously. The therapeutic agents may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

In a specific embodiment, it may be desirable to administer the therapeutic agents of the invention locally to the area in need of treatment; this may be achieved by, for example, and not by way of limitation, local infusion, by injection, or by means of an implant, said implant being of a porous, non-porous, or gelatinous material, including membranes, such as sialastic membranes, or fibers.

In yet another embodiment, the therapeutic agent can be delivered in a controlled release or sustained release system. In one embodiment, a pump may be used to achieve controlled or sustained release (see Langer, supra; Sefton, 1987, CRC Crit. Ref. Biomed. Eng. 14:20; Buchwald et al., 1980, Surgery 88:507; Saudek et al., 1989, N. Engl. J. Med. 321:574). In another embodiment, polymeric materials can be used to achieve controlled or sustained release of the compound identified by the methods of the invention (see, e.g., Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, 1983, J. Macromol. Sci. Rev. Macromol. Chem. 23:61; see also Levy et al., 1985, Science 228:190; During et al., 1989, Ann. Neurol. 25:351; Howard et al., 1989, J. Neurosurg. 7 1:105); U.S. Pat. Nos. 5,679,377; 5,916,597; 5,912,015; 5,989,463; 5,128,326; International Publication Nos. WO 99/15154 and WO 99/20253. Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In a preferred embodiment, the polymer used in a sustained release formulation is inert, free of leachable impurities, stable on storage, sterile, and biodegradable. In yet another embodiment, a controlled or sustained release system can be placed in proximity of the therapeutic target, thus requiring only a fraction of the systemic dose (see, e.g., Goodson, in Medical Applications of Controlled Release, supra, vol. 2, pp. 115-138 (1984)). Controlled release systems are discussed in the review by Langer (1990, Science 249:1527-1533). Any technique known to one of skill in the art can be used to produce sustained release formulations comprising one or more prophylacetic or therapeutic agents of the invention. See, e.g., U.S. Pat. No. 4,526,938; International Publication Nos. WO 91/05548 and WO 96/20698; Ning et al., 1996, Radiotherapy & Oncology 39:179-189; Song et al., 1995, PDA Journal of Pharmaceutical Science & Technology 50:372-397; Cleek et al., 1997, Pro. Int'l. Symp. Control. Rel. Bioact. Mater. 24:853-854; and Lam et al., 1997, Proc. Int'l. Symp. Control Rel. Bioact. Mater. 24:759-760, each of which is incorporated herein by reference in its entirety.

5.2.1.2 Formulations

Pharmaceutical compositions for use in accordance with the present invention may be formulated in conventional manner using one or more physiologically acceptable carriers or excipients. Cancer therapies and their dosages, routes of administration and recommended usage are known in the art and have been described in such literature as the Physician's Desk Reference (59th ed., 2005). The uPAR-binding molecule-drug conjugate of the invention may be formulated for administration by various routes. Methods of administering a prophylacetic or therapeutic agent of the invention include, but are not limited to, parenteral (e.g., intradermal, intramuscular, intraperitoneal, intravenous and subcutaneous), intratumoral, epidural, and mucosal (e.g., intranasal, inhaled, and oral routes) administration. In a specific embodiment, prophylacetic or therapeutic agents of the invention are administered intramuscularly, intravenously, or subcutaneously. The prophylacetic or therapeutic agents may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

In preferred embodiment, the conjugate is administered via inhalation or insufflation (either through the mouth or the nose) or oral, parenteral, intratumoral, or mucosal (such as buccal, vaginal, rectal, sublingual) administration. In a specific embodiment local administration is used. In another embodiment, parenteral administration is used.

In a specific embodiment, the term “pharmaceutically acceptable” means approved by a regulatory agency of the Federal or a state government or listed in the U.S. Pharmacopeias or other generally recognized pharmacopeias for use in animals, and more particularly in humans.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. The term “carrier” refers to a diluent, adjuvant (e.g., Freund's adjuvant (complete and incomplete), excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound. For buccal administration the compositions may take the form of tablets or lozenges formulated in conventional manner. For administration by inhalation, the therapeutic agents for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

The therapeutic agents may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The prophylacetic or therapeutic agents may also be formulated in rectal compositions such as suppositories or retention enemas, e.g., containing conventional suppository bases such as cocoa butter or other glycerides.

In addition to the formulations described previously, the therapeutic agents may also be formulated as a depot preparation. Such long acting formulations may be administered by implantation (for example subcutaneously or intramuscularly) or by intramuscular injection. Thus, for example, the therapeutic agents may be formulated with suitable polymeric or hydrophobic materials (for example as an emulsion in an acceptable oil) or ion exchange resins, or as sparingly soluble derivatives, for example, as a sparingly soluble salt.

The invention also provides that a therapeutic agent is packaged in a hermetically sealed container such as an ampoule or sachette indicating the quantity. In one embodiment, the therapeutic agent is supplied as a dry sterilized lyophilized powder or water free concentrate in a hermetically sealed container and can be reconstituted, e.g., with water or saline to the appropriate concentration for administration to a subject. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration. The pharmaceutical compositions of the invention can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

The formulation and administration of various drug are known in the art and often described in the Physicians' Desk Reference, 59^{th} ed. 2005. Radiation therapy agents such as radioactive isotopes can be given orally as liquids in capsules or as a drink. Radioactive isotopes can also be formulated for intravenous injections. The skilled oncologist can determine the preferred formulation and route of administration.

The compositions may, if desired, be presented in a pack or dispenser device that may contain one or more unit dosage forms containing the active ingredient. The pack may for example comprise metal or plastic foil, such as a blister pack. The pack or dispenser device may be accompanied by instructions for administration.

In a specific embodiment, it may be desirable to administer the prophylacetic or therapeutic agents of the invention locally to the area in need of treatment; this may be achieved by, for example, and not by way of limitation, local infusion, by injection, or by means of an implant, said implant being of a porous, non-porous, or gelatinous material, including membranes, such as sialastic membranes, or fibers.

In yet another embodiment, the prophylacetic or therapeutic agent can be delivered in a controlled release or sustained release system. In one embodiment, a pump may be used to achieve controlled or sustained release (see Langer, supra; Sefton, 1987, CRC Crit. Ref Biomed. Eng. 14:20; Buchwald et al., 1980, Surgery 88:507; Saudek et al., 1989, N. Engl. J. Med. 321:574). In another embodiment, polymeric materials can be used to achieve controlled or sustained release of the compound identified by the methods of the invention (see, e.g., Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, 1983, J. Macromol. Sci. Rev. Macromol. Chem. 23:61; see also Levy et al., 1985, Science 228:190; During et al., 1989, Ann. Neurol. 25:351; Howard et al., 1989, J. Neurosurg. 7 1:105); U.S. Pat. Nos. 5,679,377; 5,916,597; 5,912,015; 5,989,463; 5,128,326; International Publication Nos. WO 99/15154 and WO 99/20253. Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In a preferred embodiment, the polymer used in a sustained release formulation is inert, free of leachable impurities, stable on storage, sterile, and biodegradable. In yet another embodiment, a controlled or sustained release system can be placed in proximity of the prophylacetic or therapeutic target, thus requiring only a fraction of the systemic dose (see, e.g., Goodson, in Medical Applications of Controlled Release, supra, vol. 2, pp. 115-138 (1984)).

Controlled release systems are discussed in the review by Langer (1990, Science 249:1527-1533). Any technique known to one of skill in the art can be used to produce sustained release formulations comprising one or more prophylacetic or therapeutic agents of the invention. See, e.g., U.S. Pat. No. 4,526,938; International Publication Nos. WO 91/05548 and WO 96/20698; Ning et al., 1996, Radiotherapy & Oncology 39:179-189; Song et al., 1995, PDA Journal of Pharmaceutical Science & Technology 50:372-397; Cleek et al., 1997, Pro. Int'l. Symp. Control. Rel. Bioact. Mater. 24:853-854; and Lam et al., 1997, Proc. Int'l. Symp. Contro.l Rel. Bioact. Mater. 24:759-760, each of which is incorporated herein by reference in its entirety.

5.2.1.3 Dosage and Frequency of Administration

The amount of a therapeutic agent or a composition of the invention which will be effective in the prevention, treatment, management, and/or amelioration of a cancer (e.g., cancer of the breast, prostate, ovary, lung, colon, pancreas or bladder), or one or more symptoms thereof can be determined by standard clinical methods. The frequency and dosage will vary also according to factors specific for each patient depending on the specific therapies (e.g., the specific therapeutic or prophylacetic agent or a gents) administered, the severity of the disorder, disease, or condition, the route of administration, as well as age, body, weight, response, and the past medical history of the patient. For example, the dosage of a therapeutic agent or a composition of the invention which will be effective in the treatment, management, and/or amelioration of cancer, or one or more symptoms thereof can be determined by administering the composition to an animal model such as, e.g., the animal models disclosed herein or known in to those skilled in the art. In addition, in vitro assays may optionally be employed to help identify optimal dosage ranges. Suitable regimens can be selected by one skilled in the art by considering such factors and by following, for example, dosages are reported in literature and recommended in the Physicians' Desk Reference (59th ed., 2005).

Toxicity and therapeutic efficacy of a particular uPAR-binding molecule-drug conjugate can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀.

The data obtained from the cell culture assays and animal studies can be used in formulating a range of dosage for use in humans. The dosage of particular uPAR-binding molecule-drug conjugate of the invention lies preferably within a range of circulating concentrations that include the ED₅₀ with little or no toxicity. The dosage may vary within this range depending upon the dosage form employed and the route of administration utilized. A dose may be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

Exemplary doses of the uPAR-binding molecule-drug conjugate of the present invention include milligram or microgram amounts of the conjugate per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram).

Generally, the dosage of a uPAR-binding molecule-drug conjugate administered to treat a disorder involved in or mediated by uPAR-expressing cells is typically 0.1 mg/kg to 100 mg/kg of the patient's body weight, although subtherapeutic dosages may be administered when combination therapy is employed. Preferably, the dosage administered to a patient is between 0.1 mg/kg and 20 mg/kg of the patient's body weight, more preferably 1 mg/kg to 10 mg/kg of the patient's body weight. In other embodiments, the dosage of the uPAR-binding molecule-drug conjugate is 50 mg/m² to 1000 mg/m², more preferably 100 mg/m² to 750 mg/m², more preferably 200 mg/m² to 500 mg/m², and yet more preferably 300 mg/m² to 400 mg/m² of a patient's body surface area.

In certain embodiments, the dosage administered to a patient is typically 0.0001 mg/kg to 100 mg/kg of the patient's body weight. Preferably, the dosage administered to a patient is between 0.0001 mg/kg and 20 mg/kg, 0.0001 mg/kg and 10 mg/kg, 0.0001 mg/kg and 5 mg/kg, 0.0001 and 2 mg/kg, 0.0001 and 1 mg/kg, 0.0001 mg/kg and 0.75 mg/kg, 0.0001 mg/kg and 0.5 mg/kg, 0.0001 mg/kg to 0.25 mg/kg, 0.0001 to 0.15 mg/kg, 0.0001 to 0.10 mg/kg, 0.001 to 0.5 mg/kg, 0.01 to 0.25 mg/kg or 0.01 to 0.10 mg/kg of the patient's body weight. Further, the dosage and frequency of administration of conjugates of the invention may be reduced by enhancing uptake and tissue penetration of the antibodies by modifications such as, for example, lipidation.

In a specific embodiment, the dosage of the conjugates of the invention administered to treat, manage, and/or ameliorate cancer, or one or more symptoms thereof in a patient is 150 μg/kg or less, preferably 125 μg/kg or less, 100 μg/kg or less, 95 μg/kg or less, 90 μg/kg or less, 85 μg/kg or less, 80 μg/kg or less, 75 μg/kg or less, 70 μg/kg or less, 65 μg/kg or less, 60 μg/kg or less, 55 μg/kg or less, 50 μg/kg or less, 45 μg/kg or less, 40 μg/kg or less, 35 μg/kg or less, 30 μg/kg or less, 25 μg/kg or less, 20 μg/kg or less, 15 μg/kg or less, 10 μg/kg or less, 5 μg/kg or less, 2.5 μg/kg or less, 2 μg/kg or less, 1.5 μg/kg or less, 1 μg/kg or less, 0.5 μg/kg or less, or 0.5 μg/kg or less of a patient's body weight. In another embodiment, the dosage of the conjugate of the invention administered to treat, manage, and/or ameliorate cancer, or one or more symptoms thereof in a patient is a unit dose of 0.1 mg to 20 mg, 0.1 mg to 15 mg, 0.1 mg to 12 mg, 0.1 mg to 10 mg, 0.1 mg to 8 mg, 0.1 mg to 7 mg, 0.1 mg to 5 mg, 0.1 to 2.5 mg, 0.25 mg to 20 mg, 0.25 to 15 mg, 0.25 to 12 mg, 0.25 to 10 mg, 0.25 to 8 mg, 0.25 mg to 7 mg, 0.25 mg to 5 mg, 0.5 mg to 2.5 mg, 1 mg to 20 mg, 1 mg to 15 mg, 1 mg to 12 mg, 1 mg to 10 mg, 1 mg to 8 mg, 1 mg to 7 mg, 1 mg to 5 mg, or 1 mg to 2.5 mg.

In other embodiments, a subject is administered one or more doses of an effective amount of one or more conjugates of the invention (e.g., a polypeptide or antibody), wherein the dose of an effective amount achieves a serum titer of at least 0.1 μg/ml, at least 0.5 μg/ml, at least 1 μg/ml, at least 2 μg/ml, at least 5 μg/ml, at least 6 μg/ml, at least 10 μg/ml, at least 15 μg/ml, at least 20 μg/ml, at least 25 μg/ml, at least 50 μg/ml, at least 100 μg/ml, at least 125 μg/ml, at least 150 μg/ml, at least 175 μg/ml, at least 200 μg/ml, at least 225 μg/ml, at least 250 μg/ml, at least 275 μg/ml, at least 300 μg/ml, at least 325 μg/ml, at least 350 μg/ml, at least 375 μg/ml, or at least 400 μg/ml of the antibodies of the invention. In yet other embodiments, a subject is administered a dose of an effective amount of one or more conjugates of the invention to achieve a serum titer of at least 0.1 μg/ml, at least 0.5 μg/ml, at least 1 μg/ml, at least, 2 μg/ml, at least 5 μg/ml, at least 6 μg/ml, at least 10 μg/ml, at least 15 μg/ml, at least 20 μg/ml, at least 25 μg/ml, at least 50 μg/ml, at least 100 μg/ml, at least 125 μg/ml, at least 150 μg/ml, at least 175 μg/ml, at least 200 μg/ml, at least 225 μg/ml, at least 250 μg/ml, at least 275 μg/ml, at least 300 μg/ml, at least 325 μg/ml, at least 350 μg/ml, at least 375 μg/ml, or at least 400 μg/ml of the one or more conjugates of the invention and a subsequent dose of an effective amount of one or more conjugates of the invention is administered to maintain a serum titer of at least 0.1 μg/ml, 0.5 μg/ml, 1 μg/ml, at least, 2 μg/ml, at least 5 μg/ml, at least 6 μg/ml, at least 10 μg/ml, at least 15 μg/ml, at least 20 μg/ml, at least 25 μg/ml, at least 50 μg/ml, at least 100 μg/ml, at least 125 μg/ml, at least 150 μg/ml, at least 175 μg/ml, at least 200 μg/ml, at least 225 μg/ml, at least 250 μg/ml, at least 275 μg/ml, at least 300 μg/ml, at least 325 μg/ml, at least 350 μg/ml, at least 375 μg/ml, or at least 400 μg/ml. In accordance with these embodiments, a subject may be administered 1, 2, 3, 4, 5, 6, 7, 8, 9, 10, 11, 12 or more subsequent doses.

In certain embodiments, the therapeutic composition of the present invention is administered once every 3 days, preferably, once every 4 days, once every 5 days, once every 6 days, once every 7 days, once every 8 days, once every 10 days, once every two weeks, once every three weeks, or once a month.

The present invention provides methods of treating, managing, or preventing cancer or one or more symptoms thereof, said method comprising: (a) administering to a subject in need thereof one or more doses of a therapeutically effective amount of one or more conjugates of the invention; and (b) monitoring the plasma level/concentration of the said administered conjugates in said subject after administration of a certain number of doses of the said conjugates. Moreover, preferably, said certain number of doses is 1, 2, 3, 4, 5, 6, 7, 8, 9, 10, 11, or 12 doses of a therapeutically effective amount one or more therapeutic compositions of the invention.

Therapies (e.g., prophylacetic or therapeutic agents), other than the conjugates of the invention, which have been or are currently being used to treat, manage, and/or ameliorate cancer or more symptoms thereof can be administered in combination with one or more conjugates of the invention according to the methods of the invention to treat, manage, and/or ameliorate cancer or one or more symptoms thereof. Preferably, the dosages of therapeutic agents used in combination therapies of the invention are lower than those which have been or are currently being used to treat, manage, and/or ameliorate cancer or one or more symptoms thereof. The recommended dosages of agents currently used for the treatment, management, or amelioration of cancer or one or more symptoms thereof can be obtained from any reference in the art including, but not limited to, Hardman et al., eds., 2001, Goodman & Gilman's The Pharmacological Basis Of Basis Of Therapeutics, 10th ed., Mc-Graw-Hill, New York; Physicians' Desk Reference (59th ed., 2005), Medical Economics Co., Inc., Montvale, N.J., which are incorporated herein by reference in its entirety.

In various embodiments, the therapies (e.g., prophylacetic or therapeutic agents) are administered less than 5 minutes apart, less than 30 minutes apart, 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours part. In preferred embodiments, two or more therapies are administered within the same patient visit.

In certain embodiments, one or more conjugates of the invention and one or more other therapies (e.g., prophylacetic or therapeutic agents) are cyclically administered. Cycling therapy involves the administration of a first therapy (e.g., a first prophylacetic or therapeutic agent) for a period of time, followed by the administration of a second therapy (e.g., a second prophylacetic or therapeutic agent) for a period of time, optionally, followed by the administration of a third therapy (e.g., prophylacetic or therapeutic agent) for a period of time and so forth, and repeating this sequential administration, i.e., the cycle in order to reduce the development of resistance to one of the therapies, to avoid or reduce the side effects of one of the therapies, and/or to improve the efficacy of the therapies. In certain embodiments, the administration of the same conjugate of the invention may be repeated and the administrations may be separated by at least 1 day, 2 days, 3 days, 5 days, 10 days, 15 days, 30 days, 45 days, 2 months, 75 days, 3 months, or at least 6 months. In other embodiments, the administration of the same therapy (e.g., prophylacetic or therapeutic agent) other than the conjugates of the invention may be repeated and the administration may be separated by at least at least 1 day, 2 days, 3 days, 5 days, 10 days, 15 days, 30 days, 45 days, 2 months, 75 days, 3 months, or at least 6 months. The pharmaceutical compositions can be included in a container, pack, or dispenser together with instructions for administration.

### 5.3 Accumulation of uPAR-Binding Molecule-Drug Conjugate in uPAR-Expressing Cells

The uPAR-binding molecule-drug conjugate of the present invention is capable of accumulating inside a uPAR-expressing cell that results in a cytotoxic or cytostatic effect. The rate of accumulation inside a uPAR-expressing cell is the net effect of internalization of the conjugate into the cell and export of the conjugate out of the cell.

Without being bound by any theory, the uPAR-binding molecule-drug conjugate of the present invention binds to the uPAR on the uPAR-expressing cell surface. The uPAR-binding molecule-drug conjugate is internalized into the uPAR-expressing cell. In certain embodiments, the uPAR-binding molecule-drug conjugate formed a complex with uPAR and the complex is internalized into the uPAR-expressing cell. In other embodiments, the uPAR-binding molecule-drug conjugate formed a temporary complex with uPAR and the uPAR-binding molecule-drug conjugate is released into the uPAR-expressing cell. In another embodiment, the uPAR-binding molecule-drug conjugate formed a complex with uPAR in addition to other proteins, either simultaneously or consecutively, and the complex comprising uPAR-binding molecule-drug conjugate and uPAR is internalized. In other embodiments, the uPAR-binding molecule-drug conjugate first binds to PAI-1 to form a uPAR-binding molecule-drug conjugate PAI-1 complex. The uPAR-binding molecule-drug conjugate PAI-1 complex then binds to uPAR to form a uPAR-binding molecule-drug conjugate PAI-1 uPAR complex on the surface of a uPAR-expressing cell which is internalized into the cell. In another embodiment, the uPAR-binding molecule-drug conjugate PAI-1 uPAR complex further binds α₂-macroglobulin receptor/low density lipoprotein receptor-related protein.

In certain embodiments, the rate of accumulation of a uPAR-binding molecule-drug conjugate inside a uPAR-expressing cell is at least 1.5-fold, 2-fold, 5-fold, 10-fold, 20-fold, 50-fold, 200-fold, 500-fold, or 1000-fold greater than the rate of accumulation inside a uPAR-expressing cell of an unconjugated drug. In certain embodiments, the rate of accumulation of a uPAR-binding molecule-drug conjugate inside a uPAR-expressing cell is at most 2-fold, 5-fold, 10-fold, 20-fold, 50-fold, 200-fold, 500-fold, or 1000-fold greater than the rate of accumulation inside a uPAR-expressing cell of an unconjugated drug. In certain embodiments, the rate of accumulation of a uPAR-binding molecule-drug conjugate inside a uPAR-expressing cell is at least 20 to 40, 40 to 60, 60 to 80, 80 to 100, 100-200, 200-500, 500 to 1,000, 1,000 to 2,000, 2,000 to 2,500 greater than the rate of accumulation inside a uPAR-expressing cell of an unconjugated drug.

In a specific embodiment, the rate of accumulation inside a uPAR expressing cell of a uPAR-binding molecule-drug conjugate is measured by incubating a uPAR-expressing cell with isotopically labeled a uPAR-binding molecule-drug conjugate under conditions conducive to accumulation of the uPAR-binding molecule-drug conjugate inside the cell. The isotope labeling can be on the antibody, the linker, or the drug moiety of the uPAR-binding molecule-drug conjugate, but is preferably on the drug agent so that the rate can be compared to that of a similarly labeled, unconjugated drug. Subsequently, the radioactivity inside the cells is measured by any method known to the skilled artisan, such as by placing the cells into a scintillation counter. The amount of radioactivity measured is proportional to the uPAR-binding molecule-drug conjugate accumulated inside the uPAR-expressing cells.

To determine the ratio of the rates of accumulation inside a uPAR-expressing cells between a uPAR-binding molecule-drug conjugate of the invention, or the unconjugated drug, the respective rates are measured under the same conditions in parallel. The same conditions relate inter alia to the following parameters: approximately the same cell density at the beginning of the assay, the same number of cells being assayed, the same temperature, culture medium, CO₂ concentration, same period of time of the different incubation and culturing steps.

Determining the differential rate of accumulation does not require measuring and comparing absolute rates of accumulation. Rather, the relative amounts of radioactivity taken up by the uPAR-expressing cells in a given time period under similar conditions can be used as an indicator of the relative rates of accumulation of the uPAR-binding molecule-drug conjugate of the invention, or the unconjugated drug. In other embodiments, the uPAR-binding molecule-drug conjugate is labeled with a fluorescent label rather than a radioisotope. The relative rate of accumulation of the fluorescent label, for example as measured by fluorometry, can be used to determine the relative rates of uPAR-binding molecule-drug conjugate versus unconjugated drug accumulation inside the cells. In specific embodiment, the uPAR-binding molecule-drug conjugate or drug bound to the surface of the uPAR-expressing cells is removed from the cells prior to measuring the amount of fluorescent signal that has accumulated inside the cell, for example by using one or more acid washes.

### 5.4 Target Cancers

The compositions and methods of the present invention are useful for treating or preventing cancers involving or mediated by uPAR-expressing cells. Treatment of cancers, according to the methods of the present invention, is achieved by administering to a patient in need of such treatment a uPAR-binding molecule-drug conjugate of the invention.

Cancers and related disorders that can be treated, managed, or ameliorated in accordance with the invention include, but are not limited to cancers of epithelial origin, endothelial origin, etc. Non-limiting examples of such cancers include the following: leukemias, such as but not limited to, acute leukemia, acute lymphocytic leukemia, acute myelocytic leukemias, such as, myeloblastic, promyelocytic, myelomonocytic, monocytic, and erythroleukemia leukemias and myelodysplastic syndrome; chronic leukemias, such as but not limited to, chronic myelocytic (granulocytic) leukemia, chronic lymphocytic leukemia, hairy cell leukemia; polycythemia vera; lymphomas such as but not limited to Hodgkin's disease, non-Hodgkin's disease; multiple myelomas such as but not limited to smoldering multiple myeloma, nonsecretory myeloma, osteosclerotic myeloma, plasma cell leukemia, solitary plasmacytoma and extramedullary plasmacytoma; Waldenström's macroglobulinemia; monoclonal gammopathy of undetermined significance; benign monoclonal gammopathy; heavy chain disease; bone and connective tissue sarcomas such as but not limited to bone sarcoma, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangiosarcoma, neurilemmoma, rhabdomyosarcoma, synovial sarcoma; brain tumors such as but not limited to, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, primary brain lymphoma; breast cancer including but not limited to adenocarcinoma, lobular (small cell) carcinoma, intraductal carcinoma, medullary breast cancer, mucinous breast cancer, tubular breast cancer, papillary breast cancer, Paget's disease, and inflammatory breast cancer; adrenal cancer such as but not limited to pheochromocytom and adrenocortical carcinoma; thyroid cancer such as but not limited to papillary or follicular thyroid cancer, medullary thyroid cancer and anaplastic thyroid cancer; pancreatic cancer such as but not limited to, insulinoma, gastrinoma, glucagonoma, vipoma, somatostatin-secreting tumor, and carcinoid or islet cell tumor; pituitary cancers such as but limited to Cushing's disease, prolactin-secreting tumor, acromegaly, and diabetes insipius; eye cancers such as but not limited to ocular melanoma such as iris melanoma, choroidal melanoma, and cilliary body melanoma, and retinoblastoma; vaginal cancers such as squamous cell carcinoma, adenocarcinoma, and melanoma; vulvar cancer such as squamous cell carcinoma, melanoma, adenocarcinoma, basal cell carcinoma, sarcoma, and Paget's disease; cervical cancers such as but not limited to, squamous cell carcinoma, and adenocarcinoma; uterine cancers such as but not limited to endometrial carcinoma and uterine sarcoma; ovarian cancers such as but not limited to, ovarian epithelial carcinoma, borderline tumor, germ cell tumor, and stromal tumor; esophageal cancers such as but not limited to, squamous cancer, adenocarcinoma, adenoid cystic carcinoma, mucoepidermoid carcinoma, adenosquamous carcinoma, sarcoma, melanoma, plasmacytoma, verrucous carcinoma, and oat cell (small cell) carcinoma; stomach cancers such as but not limited to, adenocarcinoma, fungating (polypoid), ulcerating, superficial spreading, diffusely spreading, malignant lymphoma, liposarcoma, fibrosarcoma, and carcinosarcoma; colon cancers; rectal cancers; liver cancers such as but not limited to hepatocellular carcinoma and hepatoblastoma; gallbladder cancers such as adenocarcinoma; cholangiocarcinomas such as but not limited to papillary, nodular, and diffuse; lung cancers such as non-small-cell lung cancer, squamous cell carcinoma (epidermoid carcinoma), adenocarcinoma, large-cell carcinoma and small-cell lung cancer; testicular cancers such as but not limited to germinal tumor, seminoma, anaplastic, classic (typical), spermatocytic, nonseminoma, embryonal carcinoma, teratoma carcinoma, choriocarcinoma (yolk-sac tumor), prostate cancers such as but not limited to, adenocarcinoma, leiomyosarcoma, and rhabdomyosarcoma; penal cancers; oral cancers such as but not limited to squamous cell carcinoma; basal cancers; salivary gland cancers such as but not limited to adenocarcinoma, mucoepidermoid carcinoma, and adenoidcystic carcinoma; pharynx cancers such as but not limited to squamous cell cancer, and verrucous; skin cancers such as but not limited to, basal cell carcinoma, squamous cell carcinoma and melanoma, superficial spreading melanoma, nodular melanoma, lentigo malignant melanoma, acral lentiginous melanoma; kidney cancers such as but not limited to renal cell carcinoma, adenocarcinoma, hypernephroma, fibrosarcoma, transitional cell cancer (renal pelvis and/or uterer); Wilms' tumor; bladder cancers such as but not limited to transitional cell carcinoma, squamous cell cancer, adenocarcinoma, carcinosarcoma. In addition, cancers include myxosarcoma, osteogenic sarcoma, endotheliosarcoma, lymphangioendotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma and papillary adenocarcinomas (for a review of such disorders, see Fishman et al., 1985, Medicine, 2d ed., J.B. Lippincott Co., Philadelphia and Murphy et al., 1997, Informed Decisions: The Complete Book of Cancer Diagnosis, Treatment, and Recovery, Viking Penguin, Penguin Books U.S.A., Inc., United States of America).

Accordingly, the methods and compositions of the invention are also useful in the treatment of a variety of cancers or other abnormal proliferative diseases, including (but not limited to) the following: carcinoma, including that of the bladder, breast, colon, kidney, liver, lung, ovary, pancreas, stomach, cervix, thyroid and skin; including squamous cell carcinoma; hematopoietic tumors of lymphoid lineage, including leukemia, acute lymphocytic leukemia, acute lymphoblastic leukemia, B-cell lymphoma, T-cell lymphoma, Burkitt's lymphoma; hematopoietic tumors of myeloid lineage, including acute and chronic myelogenous leukemias and promyelocytic leukemia; tumors of mesenchymal origin, including fibrosarcoma and rhabdomyosarcoma; other tumors, including melanoma, seminoma, tetratocarcinoma, neuroblastoma and glioma; tumors of the central and peripheral nervous system, including astrocytoma, neuroblastoma, glioma, and schwannomas; tumors of mesenchymal origin, including fibrosarcoma, rhabdomyosarcoma, and osteosarcoma; and other tumors, including melanoma, xeroderma pigmentosum, keratoctanthoma, seminoma, thyroid follicular cancer and teratocarcinoma. It is also contemplated that cancers caused by aberrations in apoptosis would also be treated by the methods and compositions of the invention. Such cancers may include but not be limited to follicular lymphomas, carcinomas with p53 mutations, hormone dependent tumors of the breast, prostate and ovary, and precancerous lesions such as familial adenomatous polyposis, and myelodysplastic syndromes.

In preferred embodiments, the methods of the present invention are useful for the treatment of cancers of the liver, spleen, lymph nodes, breast, cervix, uterus, ovary, prostate, stomach, colon, lung, brain, kidney, bladder or soft tissues.

### 5.5 Kits

The invention also provides a pharmaceutical pack or kit comprising one or more containers filled with a uPAR-binding molecule-drug conjugate of the invention and optionally one or more pharmaceutical carriers. Optionally associated with such container(s) can be a notice in the form prescribed by a governmental agency regulating the manufacture, use or sale of pharmaceuticals or biological products, which notice reflects approval by the agency of manufacture, use or sale for human administration.

In yet other embodiments, the invention further provides a kit comprising in a first container, an anti-uPAR antibody, and in a second container, doxorubicin, wherein upon conjugation of the anti-uPAR antibody and doxorubicin, the resulting conjugate has a rate of accumulation in a uPAR-expressing cell that is at least 20 to 40, 40 to 60, 60 to 80, 80 to 100, 100-200, 200-500, 500 to 1,000, 1,000 to 2,000, 2,000 to 2,500 folds greater than the rate of accumulation of doxorubicin in a uPAR-expressing cell of the same cell type, wherein the rates of accumulation of the anti-uPAR antibody-drug conjugate and of the unconjugated doxorubicin are measured by a method comprising: (a) culturing a population of the uPAR-expressing cell with the anti-uPAR antibody-doxorubicin conjugate; (b) culturing a population of the uPAR-expressing cell with doxorubicin, wherein the populations of the uPA-expressing cells in steps (a) and (b) are cultured under the same conditions; and (c) measuring the amount of the anti-uPAR antibody-doxorubicin conjugate and unconjugated doxorubicin accumulated in the populations of steps (a) and (b), respectively. In a preferred embodiment, the anti-uPAR antibody is Mab 3936, or an antibody or antibody fragment that binds to the epitope of uPAR which is recognized by Mab 3936.

In yet other embodiments, the invention further provides a kit comprising in a first container, a uPAR-binding molecule, in a second container, a drug, and in a third container, a linker for conjugating the uPAR-binding molecule to the drug, wherein upon conjugation of the uPAR-binding molecule and the drug via the linker, the resulting conjugate has a rate of accumulation in a uPAR-expressing cell that is at least 20 to 40, 40 to 60, 60 to 80, 80 to 100, 100-200, 200-500, 500 to 1,000, 1,000 to 2,000, 2,000 to 2,500 folds greater than the rate of accumulation of an unconjugated drug in the uPAR-expressing cell, and wherein the rates of accumulation of the conjugate and of the unconjugated drug are measured by a method comprising: (a) culturing a population of the uPAR-expressing cell with the conjugate; (b) culturing a population of the uPAR-expressing cell with the unconjugated drug, wherein the populations of steps (a) and (b) are cultured under the same conditions; and (c) measuring the amount of the conjugate and unconjugated drug accumulated in the populations of steps (a) and (b), respectively.

In one embodiment, a kit comprises a uPAR-binding molecule-drug conjugate of the invention. In other embodiments, a kit of the invention comprises components (e.g., antibody, linker and/or drug) for manufacturing a conjugate of the invention. A kit of the invention may optionally further comprise a pharmaceutical carrier.

Therapeutic kits comprising the components of the uPAR-binding molecule drug-conjugate may be separately provided in one or more kit of the present invention. For example, the uPAR-binding molecule such as an antibody may be provided in a container separate from the therapeutic agent. In another embodiment, the uPAR-binding molecule may be provided in the same container as the therapeutic agent. The kit may further contain a linker or other components for attaching the uPAR-binding molecule to the therapeutic agent. The therapeutic kits may also contain other compounds (e.g., drugs, natural products, hormones or antagonists, anti-angiogenesis agents or inhibitors, apoptosis-inducing agents or chelators) or pharmaceutical compositions of these other compounds.

Therapeutic kits of the present invention may include components of the conjugates or conjugates packaged for use in combination with the co-administration of a second pharmaceutical composition (preferably, a chemotherapeutic agent, a natural product, a hormone or antagonist, an anti-angiogenesis agent or inhibitor, an apoptosis-inducing agent or a chelator).

Therapeutic kits of the present invention may contain one or more liquid solutions, preferably, an aqueous solution, more preferably, a sterile aqueous solution of the components of the conjugates or the conjugates. The components of the conjugates or the conjugates in the kit may also be provided as solids, which may be converted into liquids by addition of suitable solvents, which are preferably provided in another distinct container.

In other embodiments, the kits of the present invention are detection, diagnostic, monitoring, or prognostic kits.

The invention provides kits useful for monitoring the efficacy of one or more therapies that a subject is undergoing using the uPAR-binding molecule drug-conjugates of the invention.

The container of the kit of the present invention may be a vial, test tube, flask, bottle, syringe, or any other means of enclosing a solid or liquid. Usually, when there is more than one component, the kit will contain a second vial or other container, which allows for separate dosing. The kit may also contain another container for a pharmaceutically acceptable liquid. Preferably, a kit will contain devices (e.g., one or more needles, syringes, eye droppers, pipette, etc.), which enables handling of the components of the conjugates or administration of the therapeutic compounds of the invention.

In yet other embodiments, the invention provides a kit further comprising a notice by a regulatory agency indicating approval for manufacture, use or sale of the conjugate for human administration.

### 5.6 Combination Therapy for Treatment of Cancers

The uPAR-binding molecule-drug conjugate of the invention can be administered in combination with surgery, standard and experimental chemotherapies, hormonal therapies, biological therapies/immunotherapies and/or radiation therapies for treatment or prevention of cancer. In other embodiments, the uPAR-binding molecule-drug conjugate of the invention can be administered together with irradiation or one or more drugs. Such combinatorial administration can have an additive or synergistic effect on disease parameters. The combination therapy methods of the present invention provide the advantage of being able to administer reduced doses of irradiation or drugs, including doses that may be subtherapeutic by themselves, which lower the toxic and immunosuppressive side-effects of these therapies.

For irradiation treatment, the irradiation can be gamma rays or x-rays. For a general overview of radiation therapy, see Hellman, Chapter 12: Principles of Radiation Therapy Cancer, in: Principles and Practice of Oncology, DeVita et al., eds., 2nd. ed., J.B. Lippencott Company, Philadelphia.

Useful classes of therapeutic agent which may be used in combination with the uPAR-binding molecule-drug conjugate of the invention include, but are not limited to, the following non-mutually exclusive classes of agents: alkylating agents, anthracyclines, antibiotics, antifolates, antimetabolites, antitubulin agents, auristatins, chemotherapy sensitizers, DNA minor groove binders, DNA replication inhibitors, duocarmycins, etoposides, fluorinated pyrimidines, lexitropsins, nitrosoureas, platinols, purine antimetabolites, puromycins, radiation sensitizers, steroids, taxanes, topoisomerase inhibitors, and vinca alkaloids. Individual drugs that are useful for the present invention include, but are not limited to, an androgen, anthramycin (AMC), asparaginase, 5-azacytidine, azathioprine, bleomycin, busulfan, buthionine sulfoximine, camptothecin, carboplatin, carmustine (BSNU), CC-1065, chlorambucil, cisplatin, colchicine, cyclophosphamide, cytarabine, cytidine arabinoside, cytochalasin B, dacarbazine, dactinomycin (formerly actinomycin), daunorubicin, decarbazine, docetaxel, doxorubicin, an estrogen, 5-fluorodeoxyuridine, 5-fluorouracil, gramicidin D, hydroxyurea, idarubicin, ifosfamide, irinotecan, lomustine (CCNU), mechlorethamine, melphalan, 6-mercaptopurine, methotrexate, mithramycin, mitomycin C, mitoxantrone, nitroimidazole, paclitaxel, plicamycin, procarbazine, streptozotocin, tenoposide, 6-thioguanine, thioTEPA, topotecan, vinblastine, vincristine, vinorelbine, VP-16 and VM-26.

In preferred embodiments, the chemotherapeutic agent of a uPAR-binding molecule-drug conjugate of the invention is a podophyllotoxin, a taxane, a baccatin derivative, a cryptophycin, a maytansinoid, a combretastatin, or a dolastatin. In specific embodiments, the chemotherapeutic agent is vindesine, camptothecin, paclitaxel, docetaxel, epothilone A, epothilone B, nocodazole, colchicine, colcimid, estramustine, cemadotin, discodermolide, maytansine, DM-1, auristatin E-FP, auristatin E, auristatine EB, monomethyl auristatin E or eleutherobin.

In specific embodiments, the radioactive label is ⁹⁰Y, ¹¹¹In, ²¹¹At, ¹³¹I, ²¹²Bi, ²¹³Bi ²²⁵Ac, ¹⁸⁶Re, ¹⁸⁸Re, ¹⁰⁹Pd, ⁶⁷Cu, ⁷⁷Br, ¹⁰⁵Rh, ¹⁹⁸Au, ¹⁹⁹Au or ²¹²Pb.

In a specific embodiment, a uPAR-binding molecule-drug conjugate of the invention is administered concurrently with radiation therapy or one or more drugs. In another specific embodiment, chemotherapy or radiation therapy is administered prior or subsequent to administration of a nucleic acid or protein of the invention, by at least an hour and up to several months, for example, at least an hour, five hours, 12 hours, a day, a week, a month, or three months, prior or subsequent to administration of the uPAR-binding molecule-drug conjugate of the invention of the invention.

In a specific embodiment in which a uPAR-binding molecule-drug conjugate of the invention is further conjugated to a prodrug converting enzyme, the uPAR-binding molecule-drug conjugate of the invention is administered with a prodrug. As used herein, the term “prodrug” refers to a drug which is in an inactive (or significantly less active) form. The prodrug can be metabolized in the body (in vivo) into the active form. In specific embodiments, the prodrug is a derivative of a biologically active material that can hydrolyze, oxidize, or otherwise react under biological conditions (in vitro or in vivo). Although a prodrug may become active when such reactions occur, the prodrug may have certain activity in its unreacted form. Examples of prodrugs that are useful in this invention include but are not limited to analogs or derivatives of a drug that comprise biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Other examples of prodrugs include derivatives of a drug that comprise —NO, —NO₂, —ONO, or —ONO₂ moieties. Prodrugs can typically be prepared using well-known methods, such as those described by Burger's Medicinal Chemistry and Drug Discovery (1995) pp. 172-178, 949-982 (Manfred E. Wolff (ed.), (5th ed.) and Design of Prodrugs (H. Bundgaard (ed.), Elsevier, New York 1985). Administration of the prodrug can be concurrent with administration of the uPAR-binding molecule-drug conjugate of the invention, or, more preferably, follows the administration of the uPAR-binding molecule-drug conjugate by at least an hour to up to one week, for example, about five hours, 12 hours, or a day.

Additionally, combination therapy may include administration of an agent that targets a receptor or receptor complex other than uPAR on the surface of the cancerous cells. An example of such an agent is a second, non-uPAR binding molecule that binds to the surface of a cancerous cell.

In certain embodiments, the method further comprises administering to the subject a cytotoxic or cytostatic agent. The cytotoxic or cytostatic agent is selected from the group consisting of: an alkylating agent, an anthracycline, an antibiotic, an antifolate, an antimetabolite, an antitubulin agent, an auristatin, a chemotherapy sensitizer, a DNA minor groove binder, a DNA replication inhibitor, a duocarmycin, an etoposide, a fluorinated pyrimidine, a lexitropsin, a nitrosourea, a platinol, a purine antimetabolite, a puromycin, a radiation sensitizer, a steroid, a taxane, a topoisomerase inhibitor, a vinca alkaloid, a purine antagonist, and a dihydrofolate reductase inhibitor. More specifically, the chemotherapeutic agent can be: androgen, anthramycin (AMC), asparaginase, 5-azacytidine, azathioprine, bleomycin, busulfan, buthionine sulfoximine, camptothecin, carboplatin, carmustine (BSNU), CC-1065, chlorambucil, cisplatin, colchicine, cyclophosphamide, cytarabine, cytidine arabinoside, cytochalasin B, dacarbazine, dactinomycin (formerly actinomycin), daunorubicin, decarbazine, docetaxel, doxorubicin, an estrogen, 5-fluorodeoxyuridine, 5-fluorouracil, gramicidin D, hydroxyurea, idarubicin, ifosfamide, irinotecan, lomustine (CCNU), mechlorethamine, melphalan, 6-mercaptopurine, methotrexate, mithramycin, mitomycin C, mitoxantrone, nitroimidazole, paclitaxel, plicamycin, procarbazine, streptozotocin, tenoposide, 6-thioguanine, thioTEPA, topotecan, vinblastine, vincristine, vinorelbine, VP-16, VM-26, azathioprine, mycophenolate mofetil, methotrexate, acyclovir, gancyclovir, zidovudine, vidarabine, ribavirin, azidothymidine, cytidine arabinoside, amantadine, dideoxyuridine, iododeoxyuridine, poscarnet, or trifluridine.

### 5.7 Diagnostic Uses

5.7.1 Detection and Quantitation of uPAR in Patient Samples

uPAR-binding molecule-drug conjugates of the present invention may be used in detecting and quantitating uPAR in diagnostic assays. Specifically, when the uPAR-binding molecule of the conjugate is an antibody, it can be used in an immunoassay.

The tissue or cell type to be analyzed will generally include those which are known, to express uPAR, such as, for example, cancer cells including breast cancer cells, ovarian cancer cells, lymphoid cancer cells, and metastatic forms thereof. Preferably, excised primary breast cancer tumor. The protein isolation methods employed herein may, for example, be such as those described in Harlow and Lane (Harlow, E. and Lane, D., 1988, Antibodies: A Laboratory Manual, Cold Spring Harbor Laboratory Press, Cold Spring Harbor, N.Y.).

For example, uPAR-binding molecule-drug conjugates comprising anti-uPAR antibodies, or fragments of antibodies, may be used to quantitatively measure uPAR polypeptides or naturally occurring variants thereof. The conjugates useful in the present invention may, additionally, be employed histologically, as in immunofluorescence or immunoelectron microscopy, for in situ detection and quantitation of uPAR gene products or conserved variants thereof. In situ detection and quantitation may be accomplished by removing a histological specimen from a subject, such as paraffin embedded sections of tissue, e.g., breast tissues, and applying thereto a labeled antibody of the present invention. The levels of uPAR may be measured quantitatively by counting the number of grains of label used on the sections. The conjugate is preferably applied onto a biological sample.

Since uPAR is known to be present in a cell-bound form and a free form, immunoassays for uPAR will typically comprise contacting a sample, such as a biological fluid, tissue or a tissue extract, freshly harvested cells, or lysates of cells which have been incubated in cell culture, in the presence of the conjugate of the present invention that specifically or selectively binds to uPAR, e.g., a detectably labeled conjugate capable of identifying uPAR polypeptide, and detecting the bound conjugate by any of a number of techniques well-known in the art (e.g., Western blot, ELISA, FACS).

In a specific embodiment, uPAR may be measured by the antigen level of the analytes in primary tumor tissue extracts. In a preferred embodiment, uPAR is measured by any assay method.

The biological sample may be brought in contact with and immobilized onto a solid phase support or carrier such as nitrocellulose, or other solid support which is capable of immobilizing cells, cell particles or soluble proteins. The support may then be washed with suitable buffers followed by treatment with the detectably labeled conjugate that selectively or specifically binds to the uPAR polypeptide. The solid phase support may then be washed with the buffer a second time to remove unbound conjugate. The amount of bound label on solid support may then be detected by conventional means.

By “solid phase support or carrier” is intended as any support capable of binding an antigen or an antibody. Well-known supports or carriers include: glass, polystyrene, polypropylene, polyethylene, dextran, nylon, amylases, natural and modified celluloses, polyacrylamides, gabbros, and magnetite. The nature of the carrier can be either soluble to some extent or insoluble for the purposes of the present invention. The support material may have virtually any possible structural configuration so long as the coupled molecule is capable of binding to an antigen or antibody. Thus, the support configuration may be spherical, as in a bead, or cylindrical, as in the inside surface of a test tube, or the external surface of a rod. Alternatively, the surface may be flat such as a sheet, test strip, etc. Preferred supports include polystyrene beads. Those skilled in the art will know many other suitable carriers for binding antibody or antigen, or will be able to ascertain the same by use of routine experimentation.

The uPAR-binding molecule-drug conjugate comprising an anti-uPAR antibody portion can be detectably labeled by linking the same to an enzyme and using the labeled conjugate in an enzyme immunoassay (EIA) (Voller, A., The Enzyme Linked Immunosorbent Assay (ELISA), 1978, Diagnostic Horizons 2:1, Microbiological Associates Quarterly Publication, Walkersville, Md.); Voller, A. et al., 1978, J. Clin. Pathol. 31:507-520; Butler, J. E., 1981, Meth. Enzymol. 73:482; Maggio, E. (ed.), 1980, Enzyme Immunoassay, CRC Press, Boca Raton, Fla.; Ishikawa, E. et al., (eds.), 1981, Enzyme Immunoassay, Kgaku Shoin, Tokyo). The enzyme which is bound to the conjugate will react with an appropriate substrate, preferably a chromogenic substrate, in such a manner as to produce a chemical moiety which can be detected, for example, by spectrophotometric, fluorimetric or by visual means. Enzymes which can be used to detectably label the conjugate include, but are not limited to, malate dehydrogenase, staphylococcal nuclease, delta-5-steroid isomerase, yeast alcohol dehydrogenase, alpha-glycerophosphate, dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase. The detection can be accomplished by colorimetric methods which employ a chromogenic substrate for the enzyme. Measurement of the levels of the proteins may be accomplished by visual comparison or electrical scanning calibrator of the extent of enzymatic reaction of a substrate in comparison with similarly prepared standards. Standards may be prepared from normal patient samples, or samples containing known uPAR in a subject. Alternatively, standards containing known levels of uPAR may be used to calibrate the uPAR measured using various assay systems.

uPAR may also be measured using any of a variety of other immunoassays. For example, by radioactively labeling the conjugate, it is possible to detect uPAR polypeptide through the use of a radioimmunoassay (RIA) (see, for example, Weintraub, B., Principles of Radioimmunoassays, Seventh Training Course on Radioligand Assay Techniques, The Endocrine Society, March, 1986). The radioactive isotope can be detected by such means as the use of a gamma counter or a scintillation counter or by autoradiography.

It is also possible to label the conjugate with a fluorescent compound. When the fluorescently labeled conjugate is exposed to light of the proper wave length, the amount of fluorescence can then be measured which indicates the level of the protein which the conjugate binds. Among the most commonly used fluorescent labeling compounds are: fluorescein isothiocyanate, rhodamine, phycoerythrin, phycocyanin, allophycocyanin, o-phthaldehyde, and fluorescamine.

The conjugate can also be detectably labeled using fluorescence emitting metals such as ¹⁵²Eu, or others of the lanthanide series. These metals can be attached to the conjugate using such metal chelating groups as diethylenetriaminepentacetic acid (DTPA) or ethylenediaminetetraacetic acid (EDTA).

The conjugate also can be detectably labeled by coupling it to a chemiluminescent compound. The presence of the chemiluminescent-tagged conjugate is then determined by detecting the presence of luminescence that arises during the course of a chemical reaction. Examples of particularly useful chemiluminescent labeling compounds are: luminol, isoluminol, theromatic acridinium ester, imidazole, acridinium salt, and oxalate ester.

Likewise, a bioluminescent compound may be used to label the conjugate used in the present invention. Bioluminescence is a type of chemiluminescence found in biological systems, in which a catalytic protein increases the efficiency of the chemiluminescent reaction. The level of a bioluminescent protein is determined by detecting the amount of luminescence. Important bioluminescent compounds for purposes of labeling are luciferin, luciferase, and aequorin.

The methods of the present invention involve the measurement of uPAR polypeptide in the subject and is valuable in the diagnosis of cancer in a subject so that an appropriate therapeutic treatment regimen may be implemented on the subject.

In a specific embodiment of the invention, uPAR polypeptide or in combination with other markers can be measured in any body fluid of the subject including but not limited to blood, serum, plasma, milk, urine, saliva, pleural effusions, synovial fluid, spinal fluid, tissue infiltrations and tumor infiltrates. In another embodiment the polypeptide is measured in tissue samples or cells directly. The present invention also contemplates a kit for measuring uPAR in a biological sample. The kit may further comprise instructions for interpreting the results for a patient. The results may be compared to a baseline level. This baseline level can be the amount that is present in a normal subject without cancer.

5.7.2 In Vivo Imaging of Tumors

Current diagnostic and therapeutic methods make use of antibodies to target imaging agents or therapeutic substances, e.g., to tumors. Labeled antibodies, derivatives and analogs thereof, and peptides and peptide mimetics which specifically bind to a uPAR can be used for diagnostic purposes to detect or quantify uPAR polypeptide. Thus, labeled conjugates comprising an antibody portion specific or selective for the uPAR polypeptide may be used in the methods of the invention for the in vivo imaging, measurement of uPAR, during the course of treatment of cancer in a subject.

Conjugates may be linked to chelators such as those described in U.S. Pat. No. 4,741,900 or U.S. Pat. No. 5,326,856. The conjugate-chelator complex may then be radiolabeled to provide an imaging agent for diagnosis or treatment of disease. The conjugates may also be used in the methods that are disclosed in U.S. Pat. No. 5,449,761 for creating a radiolabeled conjugate for use in imaging or radiotherapy.

In in vivo diagnostic applications, specific tissues or even specific cellular disorders, e.g., cancer, may be imaged by administration of a sufficient amount of a labeled conjugate using the methods of the instant invention. The image may be produced or recorded. The imaging may be produced or recorded on a template, such as film or autoradiograph. The imaging may also be stored in a digital form. The imaging may be stored as digital data on a computer. The imaging may be analyzed using a densitometer, a computer, etc. The data is analyzed by comparing the signal from the image to a standard background level. The background may be produced or recorded as a separate image or the same image.

A wide variety of metal ions suitable for in vivo tissue imaging have been tested and utilized clinically. For imaging with radioisotopes, the following characteristics are generally desirable: (a) low radiation dose to the patient; (b) high photon yield which permits a nuclear medicine procedure to be performed in a short time period; (c) ability to be produced in sufficient quantities; (d) acceptable cost; (e) simple preparation for administration; and (f) no requirement that the patient be sequestered subsequently. These characteristics generally translate into the following: (a) the radiation exposure to the most critical organ is less than 5 rad; (b) a single image can be obtained within several hours after infusion; (c) the radioisotope does not decay by emission of a particle; (d) the isotope can be readily detected; and (e) the half-life is less than four days (Lamb and Kramer, “Commercial Production of Radioisotopes for Nuclear Medicine,” In Radiotracers For Medical Applications, Vol. 1, Rayudu (ed.), CRC Press, Inc., Boca Raton, pp. 17-62). Preferably, the metal is technetium-99m.

By way of illustration, the targets that one may image include any solid neoplasm, certain organs such breast, lymph nodes, parathyroids, spleen and kidney, sites of inflammation or infection (e.g., macrophages at such sites), myocardial infarction or thromboses (neoantigenic determinants on fibrin or platelets), and the like evident to one of ordinary skill in the art.

As is also apparent to one of ordinary skill in the art, one may use the methods of the present invention in in vivo therapeutics (e.g., using radiotherapeutic metal complexes), especially after having diagnosed a diseased condition via the in vivo diagnostic method described above, or in in vitro diagnostic application (e.g., using a radiometal or a fluorescent metal complex).

Accordingly, a method of measuring the levels of uPAR by obtaining an image of an internal region of a subject comprises administering to a subject an effective amount of an antibody composition specific or selective for uPAR polypeptide conjugated with a metal in which the metal is radioactive, and recording the scintigraphic image obtained from the decay of the radioactive metal. Likewise, it is possible to enhance a magnetic resonance (MR) image of an internal region of a subject which comprises administering to a subject an effective amount of an antibody composition containing a metal in which the metal is paramagnetic, and recording the MR image of an internal region of the subject.

Other methods include a method of enhancing a sonographic image of an internal region of a subject comprising administering to a subject an effective amount of an antibody composition containing a metal and recording the sonographic image of an internal region of the subject. In this latter application, the metal is preferably any non-toxic heavy metal ion. A method of enhancing an X-ray image of an internal region of a subject is also provided which comprises administering to a subject an antibody composition containing a metal, and recording the X-ray image of an internal region of the subject. A radioactive, non-toxic heavy metal ion is preferred.

Labeled antibodies, derivatives and analogs thereof, and peptides and peptide mimetics which specifically bind to a uPAR can be used for diagnostic purposes to detect or monitor metastases during a course of treatment. In a preferred embodiment, the uPAR-binding molecule-drug conjugate of the invention can be used for diagnostic purposes to monitor micrometastases.

In a preferred embodiment, metastases are detected in the patient. The patient is an animal and is preferably a human.

In an embodiment, diagnosis is carried out by:

(a) administering to a subject an effective amount of a labeled uPAR-binding molecule-drug conjugate which specifically binds to a urokinase receptor;

(b) delaying detection for a time interval following the administration for permitting the labeled uPAR-binding molecule-drug conjugate to preferentially concentrate in any metastatic lesions in the subject and for unbound labeled molecule to be cleared to background level;

(c) determining background level; and

(d) detecting the labeled conjugate in the subject, such that detection of labeled conjugate above the background level indicates the presence of a metastatic lesion.

Background level can be determined by various methods including: measuring the amount of labeled conjugate in tissue which does not normally express uPAR, e.g., muscle, either in the subject being diagnosed or in a second subject not suspected of having metastatic tissue; or comparing the amount of labeled conjugate detected to a standard value previously determined for a particular system.

Depending on several variables, including the type of label used and the mode of administration, the time interval following the administering for permitting the labeled conjugate to preferentially concentrate in any metastatic lesions in the subject and for unbound labeled conjugate to be cleared to background level is 6 to 48 hours or 6 to 24 hours or 6 to 12 hours. In another embodiment, the time interval following administration is 5 to 20 days or 5 to 10 days.

In an embodiment, monitoring of the metastasis is carried out by repeating the method for diagnosing the metastasis, for example, one month after initial diagnosis, six months after initial diagnosis, one year after initial diagnosis, etc.

Presence of the labeled conjugate can be detected in the patient using methods known in the art for in vivo scanning. These methods depend upon the type of label used. Skilled artisans will be able to determine the appropriate method for detecting a particular label. Methods and devices that may be used in the diagnostic methods of the invention include, but are not limited to: computed tomography (CT), whole body scan such as position emission tomography (PET), magnetic resonance imaging (MRI), and sonography.

In a specific embodiment, the conjugate is labeled with a radioisotope and is detected in the patient using a radiation responsive surgical instrument (Thurston et al., U.S. Pat. No. 5,441,050). In another embodiment, the conjugate is labeled with a fluorescent compound and is detected in the patient using a fluorescence responsive scanning instrument.

Described herein are methods for detectably labeling conjugates capable of specifically recognizing one or more uPAR epitopes or epitopes of conserved variants or peptide fragments of a uPAR. The labeling and detection methods employed herein may, for example, be such as those described in Harlow and Lane (Harlow, E. and Lane, D., 1988, Antibodies: A Laboratory Manual, Cold Spring Harbor Laboratory Press, Cold Spring Harbor, N.Y.), which is incorporated herein by reference in its entirety.

One of the ways in which the uPAR-binding molecule-drug conjugates can be detectably labeled is by linking the same to an enzyme, such labeled conjugates can be used in an enzyme immunoassay such as ELISA (enzyme linked immunosorbent assay). The uPAR-binding molecule-drug conjugates of the invention can also be labeled prior to linking the uPAR-binding molecule to the drug, i.e., prior to the conjugate being formed. The enzyme which is bound to the conjugate will react with an appropriate substrate, preferably a chromogenic substrate, in such a manner as to produce a chemical moiety which can be detected, for example, by spectrophotometric, fluorimetric or by visual means. Enzymes which can be used to detectably label the conjugates include, but are not limited to, malate dehydrogenase, staphylococcal nuclease, delta-5-steroid isomerase, yeast alcohol dehydrogenase, α-glycerophosphate, dehydrogenase, triose phosphate isomerase, horseradish peroxidase, alkaline phosphatase, asparaginase, glucose oxidase, β-galactosidase, ribonuclease, urease, catalase, glucose-6-phosphate dehydrogenase, glucoamylase and acetylcholinesterase. The detection can be accomplished by colorimetric methods which employ a chromogenic substrate for the enzyme. Detection may also be accomplished by visual comparison of the extent of enzymatic reaction of a substrate in comparison with similarly prepared standards.

For use in the detection methods of the invention, the conjugates are preferably labeled with a radioisotope, including, but not limited to: ¹²⁵I, ¹³¹I, or ⁹⁹ mTc. Such conjugates can be detected in in vitro assays using a radioimmunoassay (RIA) or radioprobe. The radioactive isotope can be detected by such means as the use of a gamma counter or a scintillation counter or by autoradiography.

It is also possible to label the conjugates with a fluorescent compound. When the fluorescently labeled conjugate is exposed to light of the proper wavelength, its presence can then be detected due to fluorescence. Among the most commonly used fluorescent labeling compounds are: fluorescein isothiocyanate, rhodamine, phycoerythrin, phycocyanin, allophycocyanin, O-phthaldehyde and fluorescamine.

The conjugates can also be detectably labeled using fluorescence emitting metals such as ¹⁵²Eu, or others of the lanthanide series. These metals can be attached to the antibodies, derivatives and analogs thereof, and peptides using such metal chelating groups as diethylenetriaminepentacetic acid (DTPA) or ethylenediaminetetraacetic acid (EDTA).

The conjugates also can be detectably labeled by coupling to a chemiluminescent compound. The presence of the chemiluminescent-tagged peptides are then determined by detecting the presence of luminescence that arises during the course of a chemical reaction. Examples of particularly useful chemiluminescent labeling compounds are: luminol, isoluminol, theromatic acridinium ester, imidazole, acridinium salt and oxalate ester.

Likewise, a bioluminescent compound may be used to label the conjugates of the present invention. Bioluminescence is a type of chemiluminescence found in biological systems, in which a catalytic protein increases the efficiency of the chemiluminescent reaction. The presence of a bioluminescent protein is determined by detecting the presence of luminescence. Important bioluminescent compounds for purposes of labeling are luciferin, luciferase and aequorin.

### 6. EXAMPLES

The present invention is based in part on a uPAR-binding molecule-drug conjugate which is capable of binding to uPAR and being internalized into a uPAR-expressing cell. A particularly favored embodiment of the invention is a conjugate of the anti-human uPAR monoclonal antibody 3936 and an anthracyclin antibiotic, especially the anti-human uPAR monoclonal antibody 3936 conjugated with doxorubicin, or the anti-human uPAR monoclonal antibody 3936 conjugated with a doxorubicin derivative. The doxorubicin may be in the form of a salt, such as hydrochloride.

The uPAR-binding molecule-drug conjugate is effective in inhibiting the growth of cancer cells, particularly primary tumors. The present invention is also based on the fact that internalization of the uPAR-binding molecule drug-conjugate results in an enhancement of the effect of the chemotherapeutic agent and allowing delivery of the chemotherapeutic agent directly to the interior of the targeted cell, in which uPAR is expressed. This targeting effect and its tumor suppressive activity is exemplified in the following examples.

### 6.1 Example

### Effect of uPAR IgG on Rat Tumor Model

6.1.1 Materials and Methods

Cell and Cell Culture

Rat breast cancer cell line Mat B-III was obtained from American Type Culture Collection (Rockville, Md.). Mat B-III cells overexpressing uPAR (Mat B-III-uPAR) were developed as described in Xing and Rabbani, 1996, Int. J. Cancer 67: 423-429, incorporated herein by reference in its entirety. Cells were maintained in RPMI 1640 or in McCoy's 5A medium supplemented with 10% fetal bovine serum (FBS), 2 mM glutamine, 100 units/ml penicillin and 100 ng/ml streptomycin (Gibco, Grand Island, N.Y.). Cells were grown under standard tissue culture conditions at 37° C. in a humidified atmosphere containing 5% CO₂ in 75 cm² flasks or six well tissue culture plates (Archbarou et al., 1994, Cancer Res. 54:2372-2377; Xing and Rabbani, 1996, Int. J. Cancer 67:423-429).

Human uPAR IgG Radiolabeling

The monoclonal human uPAR IgG (#3936, American Diagnostica Inc., Greenwich, Conn.) or non-specific mouse IgG were labelled using the Iodogen method yielding a specific activity of 0.6-0.9 mCi/mg. Briefly, 100 μg of IgG was added to a vessel precoated with 10 μg of Iodogen (Pierce Chemical Co., Rockford, Ill.) according to the manufacturer's instructions. The reaction was allowed to proceed for 15 minutes at room temperature. The free ¹²⁵I was separated from the labelled IgGs using a Sephadex G25 gel filtration column (Pharmacia, Uppsula, Sweden) pre-equilibrated with phosphate buffered saline (PBS) containing 0.1% bovine serum albumin (BSA).

Animal Protocols

Inbred female Fischer rats weighing 200-250 g were obtained from Charles River, Inc. (St. Constant, Canada). Before inoculation, Mat B-III-uPAR tumor cells grown in serum-containing medium were washed with Hank's buffer and trypsinized for five minutes. Cells were then collected in Hank's buffer and centrifuged at 1500 rpm for 5 min. Cell pellets (1×106 cells) were resuspended in 200 μl saline and injected using one ml insulin syringes into the mammary fat pad of rats anesthetized with ethanol/Somnotal (MTC Pharmaceuticals, Cambridge, Ontario).

Tumor-bearing animals were injected with 50-100 μg/day of ruPAR IgG subcutaneously into the mammary fat pad from day 1 to day 7 post tumor cell inoculation. Control groups of tumor-bearing animals received either normal saline or 50-100 μg/day of preimmune rabbit IgG as control.

All animals were monitored for the development of tumors for 2-3 weeks post tumor cell inoculation. Tumor size in control and experimental animals was measured in two dimensions by calipers and tumor volume was calculated (Haq et al., 1993, J. Clin. Invest. 91:2416-2422). Control animals receiving pre-immune IgG and experimental animals injected with ruPAR IgG were sacrificed on day 10 or one day post tumor cell inoculation and evaluated for the presence of macroscopic metastases in various tissues.

6.1.2 Results

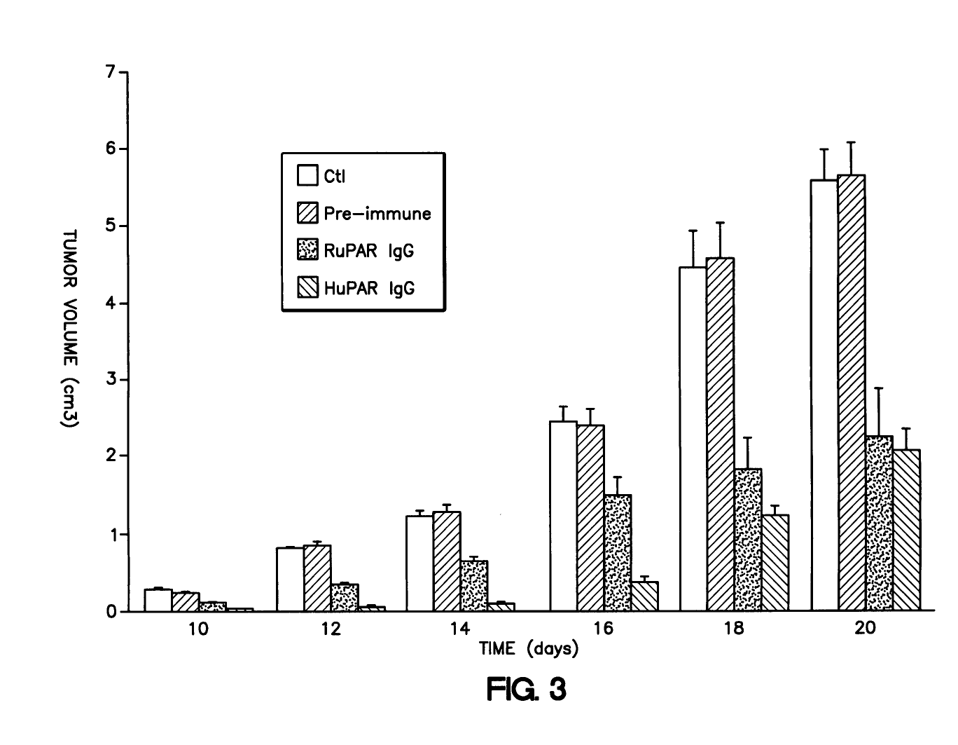

Mat B-III induced tumor in rats was studied over a 20-day period. The Mat B-III induced rats were administered with control, pre-immune serum, rabbit anti-rat uPAR IgG and mouse anti-human uPAR Mab 3936. Growth of the tumor from rats that were administered with mouse anti-human uPAR Mab 3936 was significantly suppressed as early as day 10. Growth of the tumor from rats that were administered with rabbit anti-rat uPAR shows suppression as compared to control and pre-immune serum, but not as significant as the suppression when the rat was administered with mouse anti-human uPAR Mab 3936. The tumor growth was suppressed throughout the experiment which ended at day 20. The suppression of tumor growth as compared to control and pre-immune serum indicating both rabbit anti-rat uPAR IgG and mouse anti-human uPAR IgG suppress tumor growth in rats (FIG. 3).

### 6.2 Example

### Dose Response for Mab 3936 in Rat Tumor Model

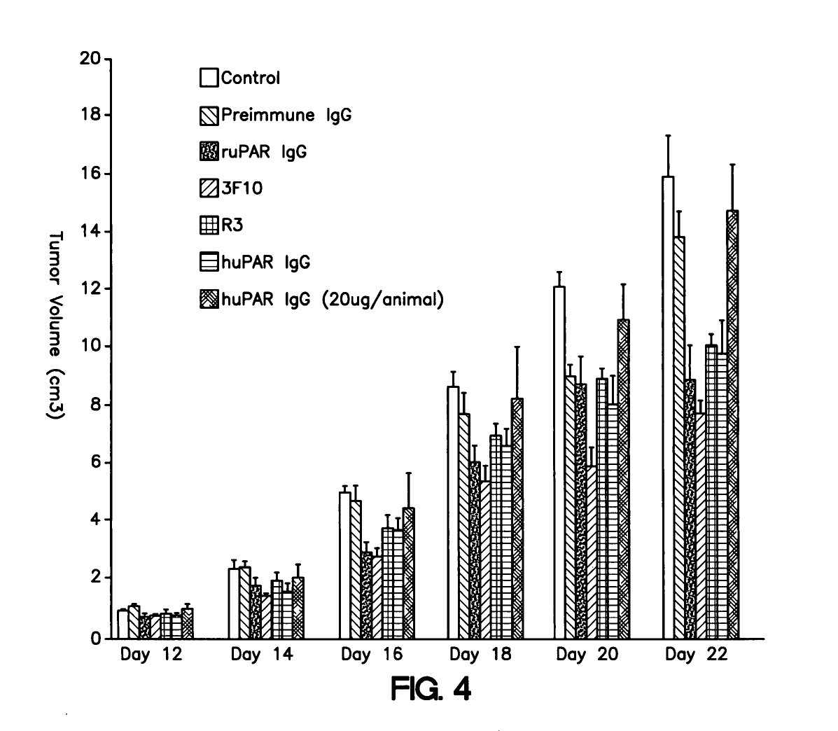

Mat B-III rat tumor model studies were conducted over a 12-22 day period. Mat B-III rats were administered daily with control (PBS), pre-immune serum (0.5 mg/kg; twice weekly), anti-rat uPAR IgG (0.5 mg/kg; twice weekly), Mab 3F10 (anti-14 kDa phospholipase A2) (0.5 mg/kg; twice weekly), Mab R3 (0.5 mg/kg twice weekly), and Mab 3936 at two dose levels: (0.5 mg/kg or 100 μg/animal) and (0.1 mg/kg or 20 μg/animal). The study exhibited reduction in tumor volume for the rats that were administered huPAR IgG Mab 3936. The higher dosage of Mab 3936 (100 μg/mL) demonstrates a more significant reduction in tumor volume in the Mat B-III rats than the lower dosage (FIG. 4).

### 6.3 Example

### Effect of Mab 3936, Doxorubicin, Mab 3936-Doxorubicin on MDA-231 GFP Tumor Growth

6.3.1 Materials and Methods

Xenograft studies using human breast carcinoma cell line MDA-MB-231.

For xenograft studies, 4 to 6 weeks old BALB/c (nu/nu) female mice were obtained from Charles River Inc. The mice weighed an average of 20 grams. Prior to inoculation, MDA-MB-231-GFP cells (Charles River) grown in serum containing culture medium were washed with Hank's balanced buffer and centrifuged at 1500 rpm for 5 min. Cell pellets (5×10⁵ cells/mice) were re-suspended in 100 μl of Matrigel (Becton Dickinson Labware, Mississauga, ON, Canada) and saline mixture (20% Matrigel) and injected into the mammary fat pads of the mice. All animals were numbered and kept separately in a temperature-controlled room on a 12 hours/12 hours light/dark schedule with food and water ad libitum. Tumors were allowed to grow to the size of 15-25 mm³ prior to drug administration. At this time, animals were randomly divided into control and experimental groups. Animals were treated with PBS, mouse IgG or various agents as described below. The tumor mass was measured in two dimensions with calipers, twice a week.

Throughout the course of these studies, all control and experimental mice were monitored for any noticeable side effects. No significant change in weight, cachexia or any other side effects were observed in the mice. Either PBS or mouse preimmune IgG (Sigma Chemicals) was administered to mice in the control group. The rest of the mice were divided into the following groups for drug administration: (1) 200 μg/mouse of doxorubicin administered via intravenous route; (2) 25 μg/mouse of doxorubicin administered via intraperitoneal route; (3) 100 mg/kg/mouse of Mab 3936 administered via intraperitoneal route; (4) 100 mg/kg/mouse of Mab 3936-doxorubicin conjugate administered via intraperitoneal route; (5) 25 mg/kg/mouse of PAI-1 administered via intraperitoneal route; (6) 25 mg/kg/mouse of PAI-doxorubicin conjugate administered via intraperitoneal route.

6.3.2 Results

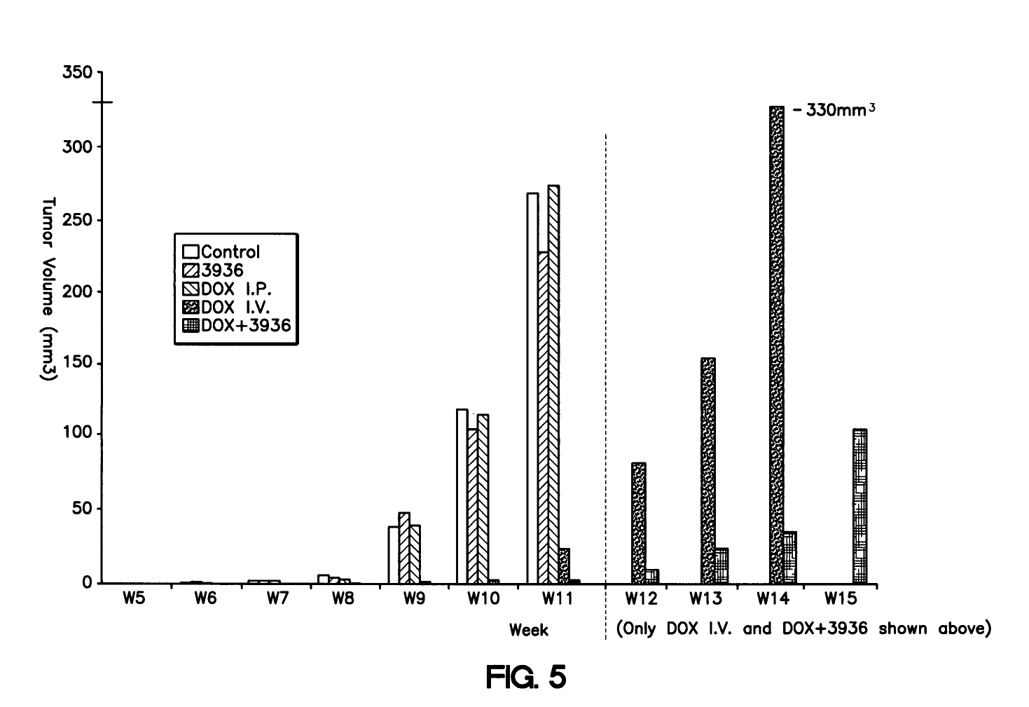

In a human breast carcinoma nude mouse xenograft study (MDA-231 GFP tumors) over a 15-week period, tumor volume of each group of mice were measured during week 8 to 15. Mice that were administered with Mab 3936-doxorubicin intraperitoneally show higher suppression of the tumor than mice administered with doxorubicin alone intraperitoneally or mice administered with Mab 3936 alone (FIG. 5). This shows that Mab 3936-doxorubicin is more effective in suppressing tumor growth than either Mab 3936 administered intraperitoneally or doxorubicin alone administered intraperitoneally. At week 11, mice that were administered doxorubicin intravenously and mice that were administered Mab 3936-doxorubicin conjugate intraperitoneally both had tumors that were less than 30 mm³. From week 12 to week 15, mice that were in the control group, or administered with Mab 3936, doxorubicin (intraperitoneal) were found to have tumors that were bigger than 350 mm³ (not shown in FIG. 5). At week 14, Mab 3936-doxorubicin conjugate was more effective in suppressing tumor growth than doxorubicin alone by a factor of 8 to 1 (330 mm³ to 40 mm³) (FIG. 5). At week 15, mice that were in the control group or administered Mab 3936, doxorubicin (intraperitoneal), and doxorubicin (intravenous) were found to have tumors that were bigger than 350 mm³ (not shown in FIG. 5). This shows that Mab 3936-doxorubicin administered intraperitoneally was significantly more effective than doxorubicin administered intravenously.

### 6.4 Example

### Effect of Mab 3936-Doxorubicin and PAI-1-Doxorubicin in Mouse Tumor Model

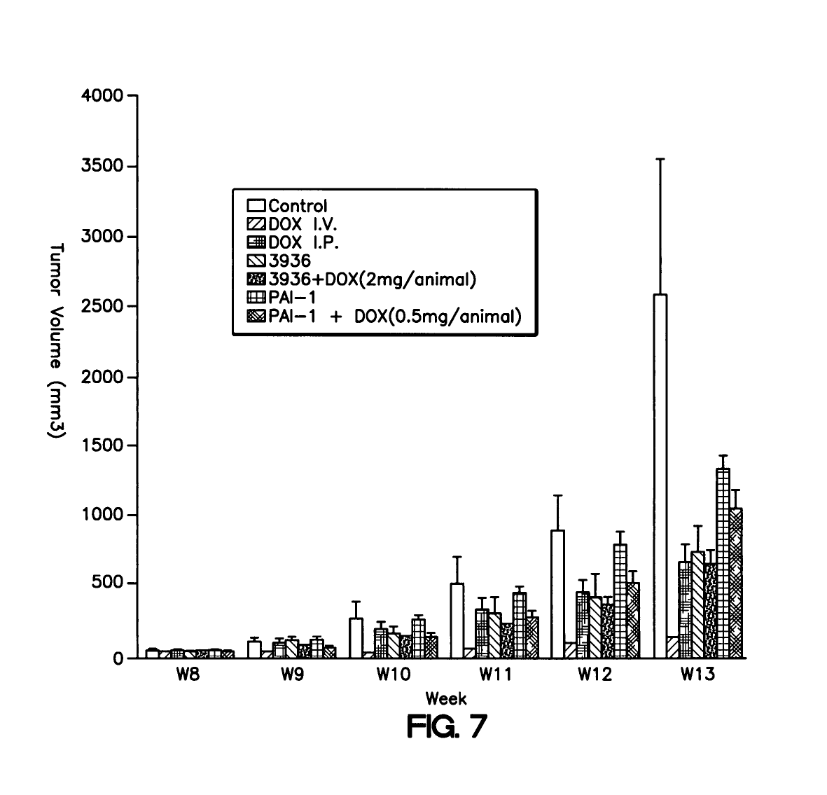

In a mouse tumor study over a 13-week period, doxorubicin (intravenous), doxorubicin (intraperitoneal), Mab 3936, Mab 3936-doxorubicin (2 mg/animal), PAI-1, and PAI-1-doxorubicin conjugate (0.5 mg/animal) were administered to the mice. The results show that Mab 3936-doxorubicin conjugate was more effective in suppressing tumor growth than PAI-1 alone or a PAI-1 doxorubicin conjugate. PAI-1 doxorubicin conjugate also show some effectiveness in suppressing tumor growth (FIG. 7).

### 6.5 Example

### Therapeutic Antibodies Conjugated to Anthracyclin Antibiotics

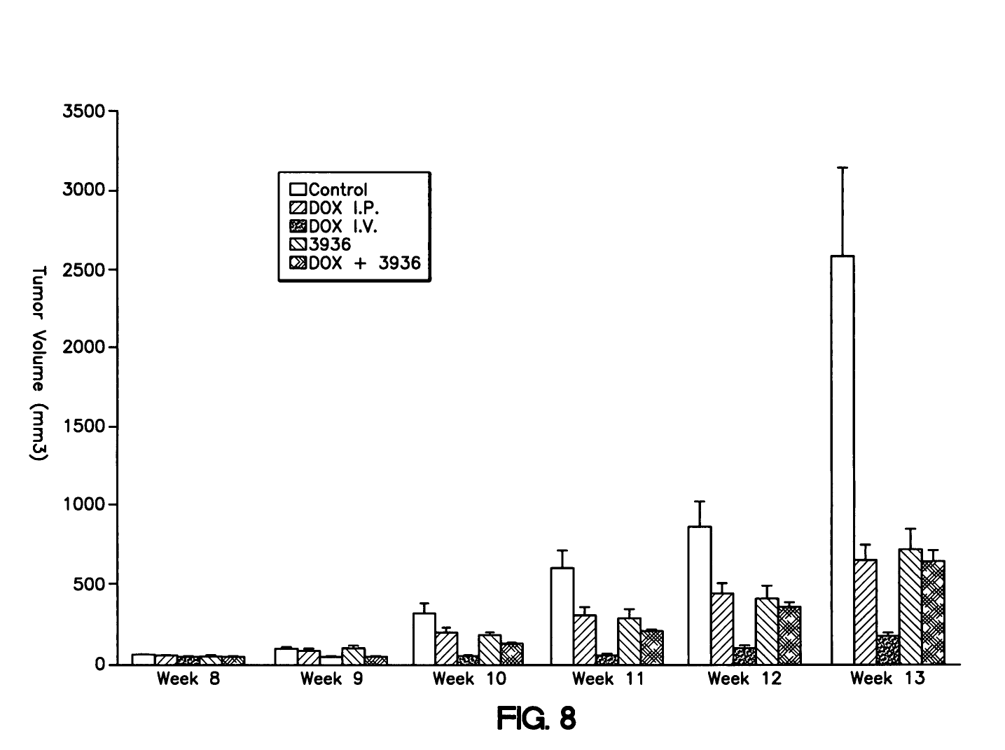

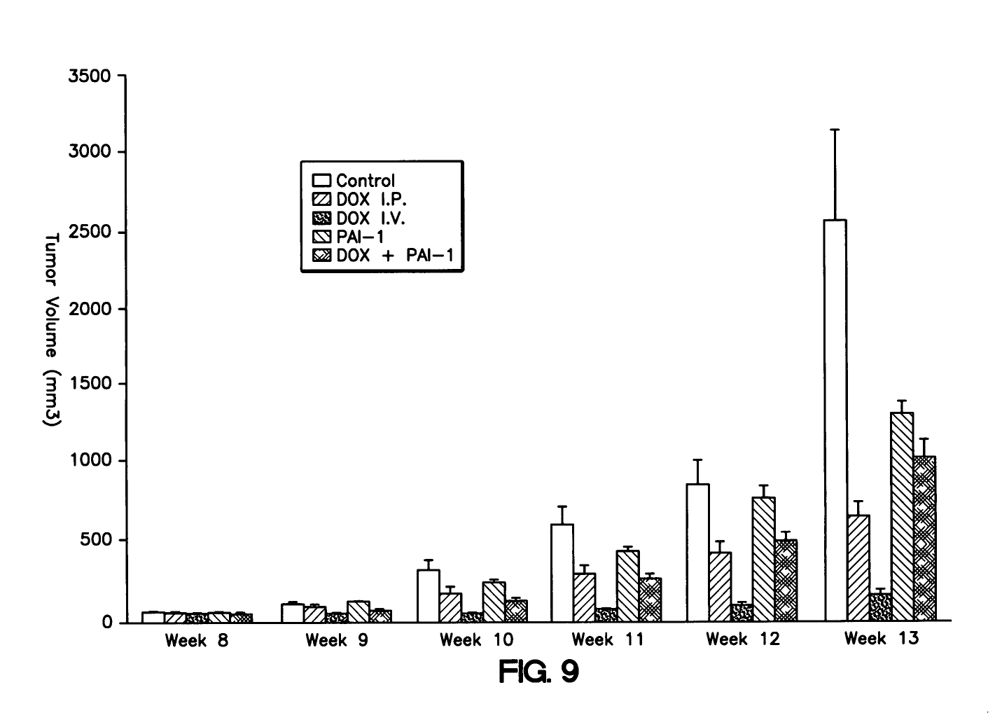

The effects of Mab 3936, doxorubicin, Mab 3936-doxorubicin conjugate, PAI-1 and PAI-1-doxorubicin conjugate on MDA-MB-231 GFP induced tumors in mice were evaluated over a 13-week period (FIG. 8 and FIG. 9). Both Mab 3936-doxorubicin conjugate and PAI-1-doxorubicin conjugate were effective in suppressing GFP tumors in mice. Administration of Mab 3936-doxorubicin conjugate was more effective in tumor growth suppression than administration of doxorubicin alone. Mab 3936-doxorubicin conjugate was more effective than PAI-1-doxorubicin conjugate in suppressing tumor growth. PAI-1-doxorubicin conjugate was also effective in suppressing tumor growth.

### 7. SPECIFIC EMBODIMENTS, CITATION OF REFERENCES

The present invention is not to be limited in scope by the specific embodiments described herein. Indeed, various modifications of the invention, in addition to those described herein, will become apparent to those skilled in the art from the foregoing description and accompanying figures. Such modifications are intended to fall within the scope of the appended claims.

Various references, including patent applications, patents, and scientific publications, are cited herein, the disclosures of which are incorporated herein by reference in their entireties.

1. A conjugate molecule comprising a first portion which comprises a uPAR-binding molecule and a second portion which comprises a drug, wherein the uPAR-binding molecule specifically binds to an epitope recognized by anti-human uPAR monoclonal antibody 3936, wherein said drug is a chemotherapeutic agent, and wherein the conjugate is capable of being internalized into a uPAR-expressing cell.

2. The conjugate molecule of claim 1 wherein the uPAR-binding molecule is an antibody, a fragment of an antibody, a peptide, peptide mimetic, derivative or analog thereof that binds specifically to uPAR.

3. The conjugate molecule of claim 2 wherein the antibody is a monoclonal antibody, a chimeric antibody, a humanized antibody, a glycosylated antibody, a multispecific antibody, a human antibody, a single chain antibody, a Fab fragment, a F(ab′) fragment, a F(ab′)₂ fragment, an Fd, a single-chain Fv, a disulfide-linked Fv, a fragment comprising a V_{L} domain, a fragment comprising a V_{H} domain, an anti-idiotype antibody, or an epitope-binding fragment.

4. The conjugate molecule of claim 3 wherein the antibody is monoclonal antibody 3936.

5. The conjugate molecule of claim 3 wherein the antibody is a humanized form of monoclonal antibody 3936.

6. The conjugate molecule of claim 1 wherein the chemotherapeutic agent is doxorubicin, morpholino-doxorubicin, cyanomorpholino-doxorubicin, its salt or a derivative thereof.

7. The conjugate molecule of claim 6 wherein the chemotherapeutic agent is doxorubicin.

8. The conjugate molecule of claim 1 wherein the conjugate is a fusion protein.

9. The conjugate molecule of claim 1 wherein the first portion and the second portion is conjugated by a linker.

10. The conjugate molecule of claim 9 wherein the linker is a biodegradable linker.

11. The conjugate molecule of claim 9 wherein the linker is a non-biodegradable linker.

12. The conjugate molecule of claim 9 wherein the linker is a peptide linker, a hydrazone linker, or a disulfide linker.

13. The conjugate molecule of claim 1 wherein the molecule has a rate of accumulation in a uPAR-expressing cell that is at least 20-40, 40-60, 60-80, 80-100, 100-200, 200-500, 500-1,000, 1,000-2,000, 2,000-2,500 folds greater than the rate of accumulation of an unconjugated form of the chemotherapeutic agent in the uPAR-expressing cell.

14. A pharmaceutical composition comprising the conjugate molecule of claim 1 and a pharmaceutically acceptable carrier.

15. The pharmaceutical composition of claim 14 wherein the uPAR-binding molecule is an antibody, a fragment of an antibody, a peptide, peptide mimetic, derivative or analog thereof that binds specifically to uPAR.

16. The pharmaceutical composition of claim 15 wherein the antibody is a monoclonal antibody, a chimeric antibody, a humanized antibody, a glycosylated antibody, a multispecific antibody, a human antibody, a single chain antibody, a Fab fragment, a F(ab′) fragment, a F(ab′)₂ fragment, a Fd, a single-chain Fv, a disulfide-linked Fv, a fragment comprising a V_{L} domain, a fragment comprising a V_{H} domain, an anti-idiotype antibody, or an epitope-binding fragment.

17. The pharmaceutical composition of claim 16 wherein the antibody is monoclonal antibody 3936.

18. The pharmaceutical composition of claim 17 wherein the antibody is a humanized form of monoclonal antibody 3936.

19. The pharmaceutical composition of claim 14 wherein the chemotherapeutic agent is doxorubicin, morpholino-doxorubicin, cyanomorpholino-doxorubicin, its salt or a derivative thereof.

20. A method of treating, ameliorating or preventing metastasis involving uPAR-expressing cells in a subject having cancer, the method comprising administering to said subject an effective amount of a conjugate molecule comprising a first portion which comprises a uPAR-binding molecule and a second portion which comprises a chemotherapeutic agent, wherein the uPAR-binding molecule specifically binds to an epitope recognized by anti-human uPAR monoclonal antibody 3936 and wherein the conjugate molecule is internalized by a uPAR-expressing cell.

21. The method of claim 20 wherein the conjugate molecule has a rate of accumulation in a uPAR-expressing cell that is at least 20-40, 40-60, 60-80, 80-100, 100-200, 200-500, 500-1,000, 1,000-2,000, 2,000-2,500 folds greater than the rate of accumulation of an unconjugated form of the chemotherapeutic agent in the uPAR-expressing cell.

22. The method of claim 20 wherein the tumor is in liver, spleen, lymph nodes, breast, cervix, uterus, ovary, prostate, stomach, colon, lung, brain, kidney, bladder, or soft tissues.

23. The method of claim 20 wherein the uPAR-binding molecule is an antibody, a fragment of an antibody, a peptide, peptide mimetic, derivative or analog thereof that binds specifically to uPAR.

24. The method of claim 23 wherein the antibody is a monoclonal antibody, a humanized chimeric antibody, a chimeric antibody, a humanized antibody, a glycosylated antibody, a multispecific antibody, a human antibody, a single chain antibody, a Fab fragment, a F(ab′) fragment, a F(ab′)₂ fragment, a Fd, a single-chain Fv, a disulfide-linked Fv, a fragment comprising a V_{L} domain, a fragment comprising a V_{H} domain, an anti-idiotype antibody, an epitope-binding fragment, or fragments thereof.

25. The method of claim 24 wherein the antibody is monoclonal antibody 3936.

26. The method of claim 25 wherein the antibody is a humanized form of monoclonal antibody 3936.

27. The method of claim 20 wherein the chemotherapeutic agent is doxorubicin, morpholino-doxorubicin, cyanomorpholino-doxorubicin, its salt or a derivative thereof.

28. The method of claim 27 wherein the chemotherapeutic agent is doxorubicin.

29. The method of claim 20 wherein the conjugate is a fusion protein.

30. The method of claim 20 wherein the first portion and the second portion is conjugated by a linker.

31. The method of claim 30 wherein the linker is a biodegradable linker.

32. The method of claim 30 wherein the linker is a non-biodegradable linker.

33. The method of claim 30 wherein the linker is a peptide linker, a hydrazone linker, or a disulfide linker.

34. The method of claim 20 wherein the molecule has a rate of accumulation in a uPAR-expressing cell that is at least 20-40, 40-60, 60-80, 80-100, 100-200, 200-500, 500-1,000, 1,000-2,000, 2,000-2,500 folds greater than the rate of accumulation of an unconjugated form of the chemotherapeutic agent in the uPAR-expressing cell.

35.-39. (canceled)

40. A kit comprising a conjugate molecule in a container, said conjugate molecule comprises a uPAR-binding molecule which immunospecifically binds to an epitope recognized by anti-human uPAR monoclonal antibody 3936, said uPAR-binding molecule is conjugated to a chemotherapeutic agent.


SEQUENCE LISTING (7 sequences)

SEQ ID NO: 1 - protein, 27 aa, Homo sapiens
VPSNCDCLNGGTCVSNKYFSNIHWCNC

SEQ ID NO: 2 - protein, 21 aa, Homo sapiens
DCLNGGTCVSNKYFSNIHWCN

SEQ ID NO: 3 - protein, 22 aa, Homo sapiens
DCLNGGTCVSNKYFSNIHWCNC

SEQ ID NO: 4 - protein, 136 aa, Homo sapiens
SNELHQVPSNCDCLNGGTCVSNKYFSNIHWCNCPKKFGGQHCEIDKSKTCYEGNGHFYRGKASTDTMGRPCLPWNSATVLQQTYHAHRSDALQLGLGKHNYCRNPDNRRRPWCYVQVGLKPLVQECMVHDCADGKK

SEQ ID NO: 5 - protein, 431 aa, Homo sapiens
MRALLARLLLCVLVVSDSKGSNELHQVPSNCDCLNGGTCVSNKYFSNIHWCNCPKKFGGQHCEIDKSKTCYEGNGHFYRGKASTDTMGRPCLPWNSATVLQQTYHAHRSDALQLGLGKHNYCRNPDNRRRPWCYVQVGLKPLVQECMVHDCADGKKPSSPPEELKFQCGQKTLRPRFKIIGGEFTTIENQPWFAAIYRRHRGGSVTYVCGGSLISPCWVISATHCFIDYPKKEDYIVYLGRSRLNSNTQGEMKFEVENLILHKDYSADTLAHHNDIALLKIRSKEGRCAQPSRTIQTICLPSMYNDPQFGTSCEITGFGKENSTDYLYPEQLKMTVVKLISHRECQQPHYYGSEVTTKMLCAADPQWKTDSCQGDSGGPLVCSLQGRMTLTGIVSWGRGCALKDKPGVYTRVSHFLPWIRSHTKEENGLAL

SEQ ID NO: 6 - DNA, 1400 bp, Homo sapiens
  CDS (47)...(1054): nucleotide sequence of uPAR
agagaagacgtgcagggaccccgcgcacaggagctgccctcgcgacatgggtcacccgccgctgctgccgctgctgctgctgctccacacctgcgtcccagcctcttggggcctgcggtgcatgcagtgtaagaccaacggggattgccgtgtggaagagtgcgccctgggacaggacctctgcaggaccacgatcgtgcgcttgtgggaagaaggagaagagctggagctggtggagaaaagctgtacccactcagagaagaccaacaggaccctgagctatcggactggcttgaagatcaccagccttaccgaggttgtgtgtgggttagacttgtgcaaccagggcaactctggccgggctgtcacctattcccgaagccgttacctcgaatgcatttcctgtggctcatcagacatgagctgtgagaggggccggcaccagagcctgcagtgccgcagccctgaagaacagtgcctggatgtggtgacccactggatccaggaaggtgaagaagggcgtccaaaggatgaccgccacctccgtggctgtggctaccttcccggctgcccgggctccaatggtttccacaacaacgacaccttccacttcctgaaatgctgcaacaccaccaaatgcaacgagggcccaatcctggagcttgaaaatctgccgcagaatggccgccagtgttacagctgcaaggggaacagcacccatggatgctcctctgaagagactttcctcattgactgccgaggccccatgaatcaatgtctggtagccaccggcactcacgaaccgaaaaaccaaagctatatggtaagaggctgtgcaaccgcctcaatgtgccaacatgcccacctgggtgacgccttcagcatgaaccacattgatgtctcctgctgtactaaaagtggctgtaaccacccagacctggatgtccagtaccgcagtggggctgctcctcagcctggccctgcccatctcagcctcaccatcaccctgctaatgactgccagactgtggggaggcactctcctctggacctaaacctgaaatccccctctctgccctggctggatccgggggacccctttgcccttccctcggctcccagccctacagacttgctgtgtgacctcaggccagtgtgccgacctctctgggcctcagttttcccagctatgaaaacagctatctcacaaagttgtgtgaagcagaagagaaaagctggaggaaggccgtgggcaatgggagagctcttgttattattaatattgttgccgctgttgtgttgttgttattaattaatattcatattatttattttatacttacataaagattttgtaccagtggaaaaaaaaaaaaaaaaaaaaaaaaaaaaaaaaaaaaa

SEQ ID NO: 7 - protein, 335 aa, Homo sapiens
MGHPPLLPLLLLLHTCVPASWGLRCMQCKTNGDCRVEECALGQDLCRTTIVRLWEEGEELELVEKSCTHSEKTNRTLSYRTGLKITSLTEVVCGLDLCNQGNSGRAVTYSRSRYLECISCGSSDMSCERGRHQSLQCRSPEEQCLDVVTHWIQEGEEGRPKDDRHLRGCGYLPGCPGSNGFHNNDTFHFLKCCNTTKCNEGPILELENLPQNGRQCYSCKGNSTHGCSSEETFLIDCRGPMNQCLVATGTHEPKNQSYMVRGCATASMCQHAHLGDAFSMNHIDVSCCTKSGCNHPDLDVQYRSGAAPQPGPAHLSLTITLLMTARLWGGTLLWT